SEQUENCE LISTING (13 sequences)

SEQ ID NO: 1 - DNA, 10735 bp, Unknown
  Severe acute respiratory syndrome coronavirus 2 (SARS-CoV-2)
agttgttagtctacgtggaccgacaagaacagtttcgaatcggaagcttgcttaacgtagttctaacagttttttattagagagcagatctctgatgaacaaccaacggaaaaagacgggtcgaccgtctttcaatatgctgaaacgcgcgagaaaccgcgtgtcaactgtttcacagttggcgaagagattctcaaaaggattgctttcaggccaaggacccatgaaattggtgatggcttttatagcattcctaagatttctagccatacctccaacagcaggaattttggctagatggggctcattcaagaagaatggagcgatcaaagtgttacggggtttcaagaaagaaatctcaaacatgttgaacataatgaacaggaggaaaagatctgtgaccatgctcctcatgctgctgcccacagccctggcgttccatctgaccacccgagggggagagccgcacatgatagttagcaagcaggaaagaggaaaatcacttttgtttaagacctctgcaggtgtcaacatgtgcacccttattgcaatggatttgggagagttatgtgaggacacaatgacctacaaatgcccccggatcactgagacggaaccagatgacgttgactgttggtgcaatgccacggagacatgggtgacctatggaacatgttctcaaactggtgaacaccgacgagacaaacgttccgtcgcactggcaccacacgtagggcttggtctagaaacaagaaccgaaacgtggatgtcctctgaaggcgcttggaaacaaatacaaaaagtggagacctgggctctgagacacccaggattcacggtgatagccctttttctagcacatgccataggaacatccatcacccagaaagggatcatttttattttgctgatgctggtaactccatccatggccatgcggtgcgtgggaataggcaacagagacttcgtggaaggactgtcaggagctacgtgggtggatgtggtactggagcatggaagttgcgtcactaccatggcaaaagacaaaccaacactggacattgaactcttgaagacggaggtcacaaaccctgccgtcctgcgcaaactgtgcattgaagctaaaatatcaaacaccaccaccgattcgagatgtccaacacaaggagaagccacgctggtggaagaacaggacacgaactttgtgtgtcgacgaacgttcgtggacagaggctggggcaatggttgtgggctattcggaaaaggtagcttaataacgtgtgctaagtttaagtgtgtgacaaaactggaaggaaagatagtccaatatgaaaacttaaaatattcagtgatagtcaccgtacacactggagaccagcaccaagttggaaatgagaccacagaacatggaacaactgcaaccataacacctcaagctcccacgtcggaaatacagctgacagactacggagctctaacattggattgttcacctagaacagggctagactttaatgagatggtgttgttgacaatgaaaaaaaaatcatggctcgtccacaaacaatggtttctagacttaccactgccttggacctcgggggcttcaacatcccaagagacttggaatagacaagacttgctggtcacatttaagacagctcatgcaaaaaagcaggaagtagtcgtactaggatcacaagaaggagcaatgcacactgcgttgactggagcgacagaaatccaaacgtctggaacgacaacaatttttgcaggacacctgaaatgcagattaaaaatggataaactgattttaaaagggatgtcatatgtaatgtgcacagggtcattcaagttagagaaggaagtggctgagacccagcatggaactgttctagtgcaggttaaatacgaaggaacagatgcaccatgcaagatccccttctcgtcccaagatgagaagggagtaacccagaatgggagattgataacagccaaccccatagtcactgacaaagaaaaaccagtcaacattgaagcggagccaccttttggtgagagctacattgtggtaggagcaggtgaaaaagctttgaaactaagctggttcaagaagggaagcagtatagggaaaatgtttgaagcaactgcccgtggagcacgaaggatggccatcctgggagacactgcatgggacttcggttctataggaggggtgttcacgtctgtgggaaaactgatacaccagatttttgggactgcgtatggagttttgttcagcggtgtttcttggaccatgaagataggaatagggattctgctgacatggctaggattaaactcaaggagcacgtccctttcaatgacgtgtatcgcagttggcatggtcacactgtacctaggagtcatggttcaggcggactcgggatgtgtaatcaactggaaaggcagagaactcaaatgtggaagcggcatttttgtcaccaatgaagtccacacctggacagagcaatataaattccaggccgactcccctaagagactatcagcggccattgggaaggcatgggaggagggtgtgtgtggaattcgatcagccactcgtctcgagaacatcatgtggaagcaaatatcaaatgaattaaaccacatcttacttgaaaatgacatgaaatttacagtggtcgtaggagacgttagtggaatcttggcccaaggaaagaaaatgattaggccacaacccatggaacacaaatactcgtggaaaagctggggaaaagccaaaatcataggagcagatgtacagaataccaccttcatcatcgacggcccaaacaccccagaatgccctgataaccaaagagcatggaacatttgggaagttgaagactatggatttggaattttcacgacaaacatatggttgaaattgcgtgactcctacactcaagtgtgtgaccaccggctaatgtcagctgccatcaaggatagcaaagcagtccatgctgacatggggtactggatagaaagtgaaaagaacgagacttggaagttggcaagagcctccttcatagaagttaagacatgcatctggccaaaatcccacactctatggagcaatggagtcctggaaagtgagatgataatcccaaagatatatggaggaccaatatctcagcacaactacagaccaggatatttcacacaaacagcagggccgtggcacttgggcaagttagaactagattttgatttatgtgaaggtaccactgttgttgtggatgaacattgtggaaatcgaggaccatctcttagaaccacaacagtcacaggaaagacaatccatgaatggtgctgtagatcttgcacgttaccccccctacgtttcaaaggagaagacgggtgctggtacggcatggaaatcagaccagtcaaggagaaggaagagaacctagttaagtcaatggtctctgcagggtcaggagaagtggacagtttttcactaggactgctatgcatatcaataatgatcgaagaggtaatgagatccagatggagcagaaaaatgctgatgactggaacattggctgtgttcctccttctcacaatgggacaattgacatggaatgatctgatcaggctatgtatcatggttggagccaacgcttcagacaagatggggatgggaacaacgtacctagctttgatggccactttcagaatgagaccaatgttcgcagtcgggctactgtttcgcagattaacatctagagaagttcttcttcttacagttggattgagtctggtggcatctgtagaactaccaaattccttagaggagctaggggatggacttgcaatgggcatcatgatgttgaaattactgactgattttcagtcacatcagctatgggctaccttgctgtctttaacatttgtcaaaacaactttttcattgcactatgcatggaagacaatggctatgatactgtcaattgtatctctcttccctttatgcctgtccacgacttctcaaaaaacaacatggcttccggtgttgctgggatctcttggatgcaaaccactaaccatgtttcttataacagaaaacaaaatctggggaaggaaaagctggcctctcaatgaaggaattatggctgttggaatagttagcattcttctaagttcacttctcaagaatgatgtgccactagctggcccactaatagctggaggcatgctaatagcatgttatgtcatatctggaagctcggccgatttatcactggagaaagcggctgaggtctcctgggaagaagaagcagaacactctggtgcctcacacaacatactagtggaggtccaagatgatggaaccatgaagataaaggatgaagagagagatgacacactcaccattctcctcaaagcaactctgctagcaatctcaggggtatacccaatgtcaataccggcgaccctctttgtgtggtatttttggcagaaaaagaaacagagatcaggagtgctatgggacacacccagccctccagaagtggaaagagcagtccttgatgatggcatttatagaattctccaaagaggattgttgggcaggtctcaagtaggagtaggagtttttcaagaaggcgtgttccacacaatgtggcacgtcaccaggggagctgtcctcatgtaccaagggaagagactggaaccaagttgggccagtgtcaaaaaagacttgatctcatatggaggaggttggaggtttcaaggatcctggaacgcgggagaagaagtgcaggtgattgctgttgaaccggggaagaaccccaaaaatgtacagacagcgccgggtaccttcaagacccctgaaggcgaagttggagccatagctctagactttaaacccggcacatctggatctcctatcgtgaacagagagggaaaaatagtaggtctttatggaaatggagtggtgacaacaagtggtacctacgtcagtgccatagctcaagctaaagcatcacaagaagggcctctaccagagattgaggacgaggtgtttaggaaaagaaacttaacaataatggacctacatccaggatcgggaaaaacaagaagataccttccagccatagtccgtgaggccataaaaagaaagctgcgcacgctagtcttagctcccacaagagttgtcgcttctgaaatggcagaggcgctcaagggaatgccaataaggtatcagacaacagcagtgaagagtgaacacacgggaaaggagatagttgaccttatgtgtcacgccactttcactatgcgtctcctgtctcctgtgagagttcccaattataatatgattatcatggatgaagcacattttaccgatccagccagcatagcagccagagggtatatctcaacccgagtgggtatgggtgaagcagctgcgattttcatgacagccactccccccggatcggtggaggcctttccacagagcaatgcagttatccaagatgaggaaagagacattcctgaaagatcatggaactcaggctatgactggatcactgatttcccaggtaaaacagtctggtttgttccaagcatcaaatcaggaaatgacattgccaactgtttaagaaagaatgggaaacgggtggtccaattgagcagaaaaacttttgacactgagtaccagaaaacaaaaaataacgactgggactatgttgtcacaacagacatatccgaaatgggagcaaacttccgagccgacagggtaatagacccgaggcggtgcctgaaaccggtaatactaaaagatggcccagagcgtgtcattctagccggaccgatgccagtgactgtggctagcgccgcccagaggagaggaagaattggaaggaaccaaaataaggaaggcgatcagtatatttacatgggacagcctctaaacaatgatgaggaccacgcccattggacagaagcaaaaatgctccttgacaacataaacacaccagaagggattatcccagccctctttgagccggagagagaaaagagtgcagcaatagacggggaatacagactacggggtgaagcgaggaaaacgttcgtggagctcatgagaagaggagatctacctgtctggctatcctacaaagttgcctcagaaggcttccagtactccgacagaaggtggtgctttgatggggaaaggaacaaccaggtgttggaggagaacatggacgtggagatctggacaaaagaaggagaaagaaagaaactacgaccccgctggctggatgccagaacatactctgacccactggctctgcgcgaattcaaagagttcgcagcaggaagaagaagcgtctcaggtgacctaatattagaaatagggaaacttccacaacatttaacgcaaagggcccagaacgccttggacaatctggttatgttgcacaactctgaacaaggaggaaaagcctatagacacgccatggaagaactaccagacaccatagaaacgttaatgctcctagctttgatagctgtgctgactggtggagtgacgttgttcttcctatcaggaaggggtctaggaaaaacatccattggcctactctgcgtgattgcctcaagtgcactgttatggatggccagtgtggaaccccattggatagcggcctctatcatactggagttctttctgatggtgttgcttattccagagccggacagacagcgcactccacaagacaaccagctagcatacgtggtgataggtctgttattcatgatattgacagtggcagccaatgagatgggattactggaaaccacaaagaaggacctggggattggtcatgcagctgctgaaaaccaccatcatgctgcaatgctggacgtagacctacatccagcttcagcctggactctctatgcagtggccacaacaattatcactcccatgatgagacacacaattgaaaacacaacggcaaatatttccctgacagctattgcaaaccaggcagctatattgatgggacttgacaagggatggccaatatcaaagatggacataggagttccacttctcgccttggggtgctattctcaggtgaacccgctgacgctgacagcggcggtattgatgctagtggctcattatgccataattggacccggactgcaagcaaaagctactagagaagctcaaaaaaggacagcagccggaataatgaaaaacccaactgtcgacgggatcgttgcaatagatttggaccctgtggtttacgatgcaaaatttgaaaaacagctaggccaaataatgttgttgatactttgcacatcacagatcctcctgatgcggaccacatgggccttgtgtgaatccatcacactagccactggacctctgactacgctttgggagggatctccaggaaaattctggaacaccacgatagcggtgtccatggcaaacatttttaggggaagttatctagcaggagcaggtctggccttttcattaatgaaatctctaggaggaggtaggagaggcacgggagcccaaggggaaacactgggagaaaaatggaaaagacagctaaaccaattgagcaagtcagaattcaacacttacaaaaggagtgggattatagaggtggatagatctgaagccaaagaggggttaaaaagaggagaaacgactaaacacgcagtgtcgagaggaacggccaaactgaggtggtttgtggagaggaaccttgtgaaaccagaagggaaagtcatagacctcggttgtggaagaggtggctggtcatattattgcgctgggctgaagaaagtcacagaagtgaaaggatacacgaaaggaggacctggacatgaggaaccaatcccaatggcaacctatggatggaacctagtaaagctatactccgggaaagatgtattctttacaccacctgagaaatgtgacaccctcttgtgtgatattggtgagtcctctccgaacccaactatagaagaaggaagaacgttacgtgttctaaagatggtggaaccatggctcagaggaaaccaattttgcataaaaattctaaatccctatatgccgagtgtggtagaaactttggagcaaatgcaaagaaaacatggaggaatgctagtgcgaaatccactctcaagaaactccactcatgaaatgtactgggtttcatgtggaacaggaaacattgtgtcagcagtaaacatgacatctagaatgctgctaaatcgattcacaatggctcacaggaagccaacatatgaaagagacgtggacttaggcgctggaacaagacatgtggcagtagaaccagaggtggccaacctagatatcattggccagaggatagagaatataaaaaatgaacacaaatcaacatggcattatgatgaggacaatccatacaaaacatgggcctatcatggatcatatgaggtcaagccatcaggatcagcctcatccatggtcaatggtgtggtgagactgctaaccaaaccatgggatgtcattcccatggtcacacaaatagccatgactgacaccacaccctttggacaacagagggtgtttaaagagaaagttgacacgcgtacaccaaaagcgaaacgaggcacagcacaaattatggaggtgacagccaggtggttatggggttttctctctagaaacaaaaaacccagaatctgcacaagagaggagttcacaagaaaagtcaggtcaaacgcagctattggagcagtgttcgttgatgaaaatcaatggaactcagcaaaagaggcagtggaagatgaacggttctgggaccttgtgcacagagagagggagcttcataaacaaggaaaatgtgccacgtgtgtctacaacatgatgggaaagagagagaaaaaattaggagagttcggaaaggcaaaaggaagtcgcgcaatatggtacatgtggttgggagcgcgctttttagagtttgaagcccttggtttcatgaatgaagatcactggttcagcagagagaattcactcagtggagtggaaggagaaggactccacaaacttggatacatactcagagacatatcaaagattccagggggaaatatgtatgcagatgacacagccggatgggacacaagaataacagaggatgatcttcagaatgaggccaaaatcactgacatcatggaacctgaacatgccctattggccacgtcaatctttaagctaacctaccaaaacaaggtagtaagggtgcagagaccagcgaaaaatggaaccgtgatggatgtcatatccagacgtgaccagagaggaagtggacaggttggaacctatggcttaaacaccttcaccaacatggaggcccaactaataagacaaatggagtctgagggaatcttttcacccagcgaattggaaaccccaaatctagccgaaagagtcctcgactggttgaaaaaacatggcaccgagaggctgaaaagaatggcaatcagtggagatgactgtgtggtgaaaccaatcgatgacagatttgcaacagccttaacagctttgaatgacatgggaaaggtaagaaaagacataccgcaatgggaaccttcaaaaggatggaatgattggcaacaagtgcctttctgttcacaccatttccaccagctgattatgaaggatgggagggagatagtggtgccatgccgcaaccaagatgaacttgtaggtagggccagagtatcacaaggcgccggatggagcttgagagaaactgcatgcctaggcaagtcatatgcacaaatgtggcagctgatgtacttccacaggagagacttgagattagcggctaatgctatctgttcagccgttccagttgattgggtcccaaccagccgcaccacctggtcgatccatgcccaccatcaatggatgacaacagaagacatgttgtcagtgtggaatagggtttggatagaggaaaacccatggatggaggacaagactcatgtgtccagttgggaagacgttccatacctaggaaaaagggaagatcaatggtgtggttccctaataggcttaacagcacgagccacctgggccaccaacatacaagtggccataaaccaagtgagaaggctcattgggaatgagaattatctagacttcatgacatcaatgaagagattcaaaaacgagagtgatcccgaaggggcactctggtaagccaactcattcacaaaataaaggaaaataaaaaatcaaacaaggcaagaagtcaggccggattaagccatagcacggtaagagctatgctgcctgtgagccccgtccaaggacgtaaaatgaagtcaggccgaaagccacggttcgagcaagccgtgctgcctgtagctccatcgtggggatgtaaaaacccgggaggctgcaaaccatggaagctgtacgcatggggtagcagactagtggttagaggagacccctcccaagacacaacgcagcagcggggcccaacaccaggggaagctgtaccctggtggtaaggactagaggttagaggagaccccccgcacaacaacaaacagcatattgacgctgggagagaccagagatcctgctgtctctacagcatcattccaggcacagaacgccaaaaaatggaatggtgctgttgaatcaacaggttct

SEQ ID NO: 2 - RNA, 1441 nt, Artificial Sequence
  Severe acute respiratory syndrome coronavirus 2 (SARS-CoV-2) derived sequence
auuaaagguuuauaccuucccagguaacaaaccaaccaacuuucgaucucuuguagaucuguucucuaaacgaacuuuaaaaucuguguggcugucacucggcugcaugcuuagugcacucacgcaguauaauuaauaacuaauuacugucguugacaggacacgaguaacucgucuaucuucugcaggcugcuuacgguuucguccguguugcagccgaucaucagcacaucuagguuucguccgggugugaccgaaagguaagauggagagccuugucccugguuucaacgagaaaacacacguccaacucaguuugccuguuuuacagguucgcgacgugcucguacguggcuuuggagacuccguggaggaggucuuaucagaggcacgucaacaucuuaaagauggcacuuguggcuuaguagaaguugaaaaaggcguuuugccucaacuugaacagcccuauguguucuagauagauaggacacuuugauggacaacagggugaaguaccaguuucuaucauuaauaacacuguuuacacaaaaguugaugguguugauguagaauuguuugaaaauaaaacaacauuaccuguuaauguagcauuugagcuuugggcuaagcgcaacauuaaaccaguaccagaggugaaaauacucaauaauuuggguguggacauugcugcuaauacugugaucugggacuacaaaagagaugcuccagcacauauaucuacuauugguguuuguucuaugacugacauagccaagaaaccaacugaaacgauuugugcaccacucacugucuuuuuugaugguagaguugauggucaaguagacuuauuuagaaaugcccguaaugguguucuuauuacagaagguaguguuaaagguuuacaaccaucuguaggucccaaacaagcuagucuuaauggagucacauuaauuggagaagccguaaaaacacaguucaauuauuauaagaaaguugaugguguuguccaacaauuaccugaaacuuacuuuacucagaguagaaauuuacaagaauuuaaacccaggagucaaauggaaauugauuucuuagaauuagcuauggaugaauucauugaacgguauaaauuagaaggcuaugccuucgaacauaucguuuauggagauuuuagucauagucaguuaggugguuuacaucuacugauuggacuagcaaucucacauagcaaucuuuaaucaguguguaacauuagggaggacuugaaagagccaccacauuuucaccgaggccacgcggaguacgaucgaguguacagugaacaaugcuagggagagcugccuauauggaagagcccuaauguguaaaauuaauuuuaguagugcuauccccaugugauuuuaauagcuucuuaggagaaugacaaaaaaaaaaaaaaaaaaaa

SEQ ID NO: 3 - RNA, 475 nt, Artificial Sequence
  Severe acute respiratory syndrome coronavirus 2 (SARS-CoV-2) derived sequence
auuaaagguuuauaccuucccagguaacaaaccaaccaacuuucgaucucuuguagaucuguucucuaaacgaacuuuaaaaucuguguggcugucacucggcugcaugcuuagugcacucacgcaguauaauuaauaacuaauuacugucguugacaggacacgaguaacucgucuaucuucugcaggcugcuuacgguuucguccguguugcagccgaucaucagcacaucuagguuucguccgggugugaccgaaagguaagauggagagccuugucccugguuucaacgagaaaacacacguccaacucaguuugccuguuuuacagguucgcgacgugcucguacguggcuuuggagacuccguggaggaggucuuaucagaggcacgucaacaucuuaaagauggcacuuguggcuuaguagaaguugaaaaaggcguuuugccucaacuugaacagcccuauguguuc

SEQ ID NO: 4 - RNA, 727 nt, Artificial Sequence
  Severe acute respiratory syndrome coronavirus 2 (SARS-CoV-2) derived sequence
gacacuuugauggacaacagggugaaguaccaguuucuaucauuaauaacacuguuuacacaaaaguugaugguguugauguagaauuguuugaaaauaaaacaacauuaccuguuaauguagcauuugagcuuugggcuaagcgcaacauuaaaccaguaccagaggugaaaauacucaauaauuuggguguggacauugcugcuaauacugugaucugggacuacaaaagagaugcuccagcacauauaucuacuauugguguuuguucuaugacugacauagccaagaaaccaacugaaacgauuugugcaccacucacugucuuuuuugaugguagaguugauggucaaguagacuuauuuagaaaugcccguaaugguguucuuauuacagaagguaguguuaaagguuuacaaccaucuguaggucccaaacaagcuagucuuaauggagucacauuaauuggagaagccguaaaaacacaguucaauuauuauaagaaaguugaugguguuguccaacaauuaccugaaacuuacuuuacucagaguagaaauuuacaagaauuuaaacccaggagucaaauggaaauugauuucuuagaauuagcuauggaugaauucauugaacgguauaaauuagaaggcuaugccuucgaacauaucguuuauggagauuuuagucauagucaguuaggugguuuacaucuacugauuggacuagc

SEQ ID NO: 5 - RNA, 228 nt, Artificial Sequence
  Severe acute respiratory syndrome coronavirus 2 (SARS-CoV-2) derived sequence
aaucucacauagcaaucuuuaaucaguguguaacauuagggaggacuugaaagagccaccacauuuucaccgaggccacgcggaguacgaucgaguguacagugaacaaugcuagggagagcugccuauauggaagagcccuaauguguaaaauuaauuuuaguagugcuauccccaugugauuuuaauagcuucuuaggagaaugacaaaaaaaaaaaaaaaaaaaa

SEQ ID NO: 6 - RNA, 1441 nt, Artificial Sequence; identical to SEQ ID NO: 2
  Severe acute respiratory syndrome coronavirus 2 (SARS-CoV-2) derived sequence

SEQ ID NO: 7 - RNA, 475 nt, Artificial Sequence; identical to SEQ ID NO: 3
  Severe acute respiratory syndrome coronavirus 2 (SARS-CoV-2) derived sequence

SEQ ID NO: 8 - RNA, 11 nt, Artificial Sequence
  Synthesized standard UAG stop codon repeat in different reading frames
uagauagauag

SEQ ID NO: 9 - DNA, 29751 bp, Unknown
  SARS coronavirus, complete genome
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgtgagctcaatggaggtgcagtcactcgctatgtcgacaacaatttctgtggcccagatgggtaccctcttgattgcatcaaagattttctcgcacgcgcgggcaagtcaatgtgcactctttccgaacaacttgattacatcgagtcgaagagaggtgtctactgctgccgtgaccatgagcatgaaattgcctggttcactgagcgctctgataagagctacgagcaccagacacccttcgaaattaagagtgccaagaaatttgacactttcaaaggggaatgcccaaagtttgtgtttcctcttaactcaaaagtcaaagtcattcaaccacgtgttgaaaagaaaaagactgagggtttcatggggcgtatacgctctgtgtaccctgttgcatctccacaggagtgtaacaatatgcacttgtctaccttgatgaaatgtaatcattgcgatgaagtttcatggcagacgtgcgactttctgaaagccacttgtgaacattgtggcactgaaaatttagttattgaaggacctactacatgtgggtacctacctactaatgctgtagtgaaaatgccatgtcctgcctgtcaagacccagagattggacctgagcatagtgttgcagattatcacaaccactcaaacattgaaactcgactccgcaagggaggtaggactagatgttttggaggctgtgtgtttgcctatgttggctgctataataagcgtgcctactgggttcctcgtgctagtgctgatattggctcaggccatactggcattactggtgacaatgtggagaccttgaatgaggatctccttgagatactgagtcgtgaacgtgttaacattaacattgttggcgattttcatttgaatgaagaggttgccatcattttggcatctttctctgcttctacaagtgcctttattgacactataaagagtcttgattacaagtctttcaaaaccattgttgagtcctgcggtaactataaagttaccaagggaaagcccgtaaaaggtgcttggaacattggacaacagagatcagttttaacaccactgtgtggttttccctcacaggctgctggtgttatcagatcaatttttgcgcgcacacttgatgcagcaaaccactcaattcctgatttgcaaagagcagctgtcaccatacttgatggtatttctgaacagtcattacgtcttgtcgacgccatggtttatacttcagacctgctcaccaacagtgtcattattatggcatatgtaactggtggtcttgtacaacagacttctcagtggttgtctaatcttttgggcactactgttgaaaaactcaggcctatctttgaatggattgaggcgaaacttagtgcaggagttgaatttctcaaggatgcttgggagattctcaaatttctcattacaggtgtttttgacatcgtcaagggtcaaatacaggttgcttcagataacatcaaggattgtgtaaaatgcttcattgatgttgttaacaaggcactcgaaatgtgcattgatcaagtcactatcgctggcgcaaagttgcgatcactcaacttaggtgaagtcttcatcgctcaaagcaagggactttaccgtcagtgtatacgtggcaaggagcagctgcaactactcatgcctcttaaggcaccaaaagaagtaacctttcttgaaggtgattcacatgacacagtacttacctctgaggaggttgttctcaagaacggtgaactcgaagcactcgagacgcccgttgatagcttcacaaatggagctatcgttggcacaccagtctgtgtaaatggcctcatgctcttagagattaaggacaaagaacaatactgcgcattgtctcctggtttactggctacaaacaatgtctttcgcttaaaagggggtgcaccaattaaaggtgtaacctttggagaagatactgtttgggaagttcaaggttacaagaatgtgagaatcacatttgagcttgatgaacgtgttgacaaagtgcttaatgaaaagtgctctgtctacactgttgaatccggtaccgaagttactgagtttgcatgtgttgtagcagaggctgttgtgaagactttacaaccagtttctgatctccttaccaacatgggtattgatcttgatgagtggagtgtagctacattctacttatttgatgatgctggtgaagaaaacttttcatcacgtatgtattgttccttttaccctccagatgaggaagaagaggacgatgcagagtgtgaggaagaagaaattgatgaaacctgtgaacatgagtacggtacagaggatgattatcaaggtctccctctggaatttggtgcctcagctgaaacagttcgagttgaggaagaagaagaggaagactggctggatgatactactgagcaatcagagattgagccagaaccagaacctacacctgaagaaccagttaatcagtttactggttatttaaaacttactgacaatgttgccattaaatgtgttgacatcgttaaggaggcacaaagtgctaatcctatggtgattgtaaatgctgctaacatacacctgaaacatggtggtggtgtagcaggtgcactcaacaaggcaaccaatggtgccatgcaaaaggagagtgatgattacattaagctaaatggccctcttacagtaggagggtcttgtttgctttctggacataatcttgctaagaagtgtctgcatgttgttggacctaacctaaatgcaggtgaggacatccagcttcttaaggcagcatatgaaaatttcaattcacaggacatcttacttgcaccattgttgtcagcaggcatatttggtgctaaaccacttcagtctttacaagtgtgcgtgcagacggttcgtacacaggtttatattgcagtcaatgacaaagctctttatgagcaggttgtcatggattatcttgataacctgaagcctagagtggaagcacctaaacaagaggagccaccaaacacagaagattccaaaactgaggagaaatctgtcgtacagaagcctgtcgatgtgaagccaaaaattaaggcctgcattgatgaggttaccacaacactggaagaaactaagtttcttaccaataagttactcttgtttgctgatatcaatggtaagctttaccatgattctcagaacatgcttagaggtgaagatatgtctttccttgagaaggatgcaccttacatggtaggtgatgttatcactagtggtgatatcacttgtgttgtaataccctccaaaaaggctggtggcactactgagatgctctcaagagctttgaagaaagtgccagttgatgagtatataaccacgtaccctggacaaggatgtgctggttatacacttgaggaagctaagactgctcttaagaaatgcaaatctgcattttatgtactaccttcagaagcacctaatgctaaggaagagattctaggaactgtatcctggaatttgagagaaatgcttgctcatgctgaagagacaagaaaattaatgcctatatgcatggatgttagagccataatggcaaccatccaacgtaagtataaaggaattaaaattcaagagggcatcgttgactatggtgtccgattcttcttttatactagtaaagagcctgtagcttctattattacgaagctgaactctctaaatgagccgcttgtcacaatgccaattggttatgtgacacatggttttaatcttgaagaggctgcgcgctgtatgcgttctcttaaagctcctgccgtagtgtcagtatcatcaccagatgctgttactacatataatggatacctcacttcgtcatcaaagacatctgaggagcactttgtagaaacagtttctttggctggctcttacagagattggtcctattcaggacagcgtacagagttaggtgttgaatttcttaagcgtggtgacaaaattgtgtaccacactctggagagccccgtcgagtttcatcttgacggtgaggttctttcacttgacaaactaaagagtctcttatccctgcgggaggttaagactataaaagtgttcacaactgtggacaacactaatctccacacacagcttgtggatatgtctatgacatatggacagcagtttggtccaacatacttggatggtgctgatgttacaaaaattaaacctcatgtaaatcatgagggtaagactttctttgtactacctagtgatgacacactacgtagtgaagctttcgagtactaccatactcttgatgagagttttcttggtaggtacatgtctgctttaaaccacacaaagaaatggaaatttcctcaagttggtggtttaacttcaattaaatgggctgataacaattgttatttgtctagtgttttattagcacttcaacagcttgaagtcaaattcaatgcaccagcacttcaagaggcttattatagagcccgtgctggtgatgctgctaacttttgtgcactcatactcgcttacagtaataaaactgttggcgagcttggtgatgtcagagaaactatgacccatcttctacagcatgctaatttggaatctgcaaagcgagttcttaatgtggtgtgtaaacattgtggtcagaaaactactaccttaacgggtgtagaagctgtgatgtatatgggtactctatcttatgataatcttaagacaggtgtttccattccatgtgtgtgtggtcgtgatgctacacaatatctagtacaacaagagtcttcttttgttatgatgtctgcaccacctgctgagtataaattacagcaaggtacattcttatgtgcgaatgagtacactggtaactatcagtgtggtcattacactcatataactgctaaggagaccctctatcgtattgacggagctcaccttacaaagatgtcagagtacaaaggaccagtgactgatgttttctacaaggaaacatcttacactacaaccatcaagcctgtgtcgtataaactcgatggagttacttacacagagattgaaccaaaattggatgggtattataaaaaggataatgcttactatacagagcagcctatagaccttgtaccaactcaaccattaccaaatgcgagttttgataatttcaaactcacatgttctaacacaaaatttgctgatgatttaaatcaaatgacaggcttcacaaagccagcttcacgagagctatctgtcacattcttcccagacttgaatggcgatgtagtggctattgactatagacactattcagcgagtttcaagaaaggtgctaaattactgcataagccaattgtttggcacattaaccaggctacaaccaagacaacgttcaaaccaaacacttggtgtttacgttgtctttggagtacaaagccagtagatacttcaaattcatttgaagttctggcagtagaagacacacaaggaatggacaatcttgcttgtgaaagtcaacaacccacctctgaagaagtagtggaaaatcctaccatacagaaggaagtcatagagtgtgacgtgaaaactaccgaagttgtaggcaatgtcatacttaaaccatcagatgaaggtgttaaagtaacacaagagttaggtcatgaggatcttatggctgcttatgtggaaaacacaagcattaccattaagaaacctaatgagctttcactagccttaggtttaaaaacaattgccactcatggtattgctgcaattaatagtgttccttggagtaaaattttggcttatgtcaaaccattcttaggacaagcagcaattacaacatcaaattgcgctaagagattagcacaacgtgtgtttaacaattatatgccttatgtgtttacattattgttccaattgtgtacttttactaaaagtaccaattctagaattagagcttcactacctacaactattgctaaaaatagtgttaagagtgttgctaaattatgtttggatgccggcattaattatgtgaagtcacccaaattttctaaattgttcacaatcgctatgtggctattgttgttaagtatttgcttaggttctctaatctgtgtaactgctgcttttggtgtactcttatctaattttggtgctccttcttattgtaatggcgttagagaattgtatcttaattcgtctaacgttactactatggatttctgtgaaggttcttttccttgcagcatttgtttaagtggattagactcccttgattcttatccagctcttgaaaccattcaggtgacgatttcatcgtacaagctagacttgacaattttaggtctggccgctgagtgggttttggcatatatgttgttcacaaaattcttttatttattaggtctttcagctataatgcaggtgttctttggctattttgctagtcatttcatcagcaattcttggctcatgtggtttatcattagtattgtacaaatggcacccgtttctgcaatggttaggatgtacatcttctttgcttctttctactacatatggaagagctatgttcatatcatggatggttgcacctcttcgacttgcatgatgtgctataagcgcaatcgtgccacacgcgttgagtgtacaactattgttaatggcatgaagagatctttctatgtctatgcaaatggaggccgtggcttctgcaagactcacaattggaattgtctcaattgtgacacattttgcactggtagtacattcattagtgatgaagttgctcgtgatttgtcactccagtttaaaagaccaatcaaccctactgaccagtcatcgtatattgttgatagtgttgctgtgaaaaatggcgcgcttcacctctactttgacaaggctggtcaaaagacctatgagagacatccgctctcccattttgtcaatttagacaatttgagagctaacaacactaaaggttcactgcctattaatgtcatagtttttgatggcaagtccaaatgcgacgagtctgcttctaagtctgcttctgtgtactacagtcagctgatgtgccaacctattctgttgcttgaccaagctcttgtatcagacgttggagatagtactgaagtttccgttaagatgtttgatgcttatgtcgacaccttttcagcaacttttagtgttcctatggaaaaacttaaggcacttgttgctacagctcacagcgagttagcaaagggtgtagctttagatggtgtcctttctacattcgtgtcagctgcccgacaaggtgttgttgataccgatgttgacacaaaggatgttattgaatgtctcaaactttcacatcactctgacttagaagtgacaggtgacagttgtaacaatttcatgctcacctataataaggttgaaaacatgacgcccagagatcttggcgcatgtattgactgtaatgcaaggcatatcaatgcccaagtagcaaaaagtcacaatgtttcactcatctggaatgtaaaagactacatgtctttatctgaacagctgcgtaaacaaattcgtagtgctgccaagaagaacaacataccttttagactaacttgtgctacaactagacaggttgtcaatgtcataactactaaaatctcactcaagggtggtaagattgttagtacttgttttaaacttatgcttaaggccacattattgtgcgttcttgctgcattggtttgttatatcgttatgccagtacatacattgtcaatccatgatggttacacaaatgaaatcattggttacaaagccattcaggatggtgtcactcgtgacatcatttctactgatgattgttttgcaaataaacatgctggttttgacgcatggtttagccagcgtggtggttcatacaaaaatgacaaaagctgccctgtagtagctgctatcattacaagagagattggtttcatagtgcctggcttaccgggtactgtgctgagagcaatcaatggtgacttcttgcattttctacctcgtgtttttagtgctgttggcaacatttgctacacaccttccaaactcattgagtatagtgattttgctacctctgcttgcgttcttgctgctgagtgtacaatttttaaggatgctatgggcaaacctgtgccatattgttatgacactaatttgctagagggttctatttcttatagtgagcttcgtccagacactcgttatgtgcttatggatggttccatcatacagtttcctaacacttacctggagggttctgttagagtagtaacaacttttgatgctgagtactgtagacatggtacatgcgaaaggtcagaagtaggtatttgcctatctaccagtggtagatgggttcttaataatgagcattacagagctctatcaggagttttctgtggtgttgatgcgatgaatctcatagctaacatctttactcctcttgtgcaacctgtgggtgctttagatgtgtctgcttcagtagtggctggtggtattattgccatattggtgacttgtgctgcctactactttatgaaattcagacgtgtttttggtgagtacaaccatgttgttgctgctaatgcacttttgtttttgatgtctttcactatactctgtctggtaccagcttacagctttctgccgggagtctactcagtcttttacttgtacttgacattctatttcaccaatgatgtttcattcttggctcaccttcaatggtttgccatgttttctcctattgtgcctttttggataacagcaatctatgtattctgtatttctctgaagcactgccattggttctttaacaactatcttaggaaaagagtcatgtttaatggagttacatttagtaccttcgaggaggctgctttgtgtacctttttgctcaacaaggaaatgtacctaaaattgcgtagcgagacactgttgccacttacacagtataacaggtatcttgctctatataacaagtacaagtatttcagtggagccttagatactaccagctatcgtgaagcagcttgctgccacttagcaaaggctctaaatgactttagcaactcaggtgctgatgttctctaccaaccaccacagacatcaatcacttctgctgttctgcagagtggttttaggaaaatggcattcccgtcaggcaaagttgaagggtgcatggtacaagtaacctgtggaactacaactcttaatggattgtggttggatgacacagtatactgtccaagacatgtcatttgcacagcagaagacatgcttaatcctaactatgaagatctgctcattcgcaaatccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcagttctagcatgctacaatggttcaccatctggtgtttatcagtgtgccatgagacctaatcataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataacattaaatgttttggcatggctgtatgctgctgttatcaatggtgataggtggtttcttaatagattcaccactactttgaatgactttaaccttgtggcaatgaagtacaactatgaacctttgacacaagatcatgttgacatattgggacctctttctgctcaaacaggaattgccgtcttagatatgtgtgctgctttgaaagagctgctgcagaatggtatgaatggtcgtactatccttggtagcactattttagaagatgagtttacaccatttgatgttgttagacaatgctctggtgttaccttccaaggtaagttcaagaaaattgttaagggcactcatcattggatgcttttaactttcttgacatcactattgattcttgttcaaagtacacagtggtcactgtttttctttgtttacgagaatgctttcttgccatttactcttggtattatggcaattgctgcatgtgctatgctgcttgttaagcataagcacgcattcttgtgcttgtttctgttaccttctcttgcaacagttgcttactttaatatggtctacatgcctgctagctgggtgatgcgtatcatgacatggcttgaattggctgacactagcttgtctggttataggcttaaggattgtgttatgtatgcttcagctttagttttgcttattctcatgacagctcgcactgtttatgatgatgctgctagacgtgtttggacactgatgaatgtcattacacttgtttacaaagtctactatggtaatgctttagatcaagctatttccatgtgggccttagttatttctgtaacctctaactattctggtgtcgttacgactatcatgtttttagctagagctatagtgtttgtgtgtgttgagtattacccattgttatttattactggcaacaccttacagtgtatcatgcttgtttattgtttcttaggctattgttgctgctgctactttggccttttctgtttactcaaccgttacttcaggcttactcttggtgtttatgactacttggtctctacacaagaatttaggtatatgaactcccaggggcttttgcctcctaagagtagtattgatgctttcaagcttaacattaagttgttgggtattggaggtaaaccatgtatcaaggttgctactgtacagtctaaaatgtctgacgtaaagtgcacatctgtggtactgctctcggttcttcaacaacttagagtagagtcatcttctaaattgtgggcacaatgtgtacaactccacaatgatattcttcttgcaaaagacacaactgaagctttcgagaagatggtttctcttttgtctgttttgctatccatgcagggtgctgtagacattaataggttgtgcgaggaaatgctcgataaccgtgctactcttcaggctattgcttcagaatttagttctttaccatcatatgccgcttatgccactgcccaggaggcctatgagcaggctgtagctaatggtgattctgaagtcgttctcaaaaagttaaagaaatctttgaatgtggctaaatctgagtttgaccgtgatgctgccatgcaacgcaagttggaaaagatggcagatcaggctatgacccaaatgtacaaacaggcaagatctgaggacaagagggcaaaagtaactagtgctatgcaaacaatgctcttcactatgcttaggaagcttgataatgatgcacttaacaacattatcaacaatgcgcgtgatggttgtgttccactcaacatcataccattgactacagcagccaaactcatggttgttgtccctgattatggtacctacaagaacacttgtgatggtaacacctttacatatgcatctgcactctgggaaatccagcaagttgttgatgcggatagcaagattgttcaacttagtgaaattaacatggacaattcaccaaatttggcttggcctcttattgttacagctctaagagccaactcagctgttaaactacagaataatgaactgagtccagtagcactacgacagatgtcctgtgcggctggtaccacacaaacagcttgtactgatgacaatgcacttgcctactataacaattcgaagggaggtaggtttgtgctggcattactatcagaccaccaagatctcaaatgggctagattccctaagagtgatggtacaggtacaatttacacagaactggaaccaccttgtaggtttgttacagacacaccaaaagggcctaaagtgaaatacttgtacttcatcaaaggcttaaacaacctaaatagaggtatggtgctgggcagtttagctgctacagtacgtcttcaggctggaaatgctacagaagtacctgccaattcaactgtgctttccttctgtgcttttgcagtagaccctgctaaagcatataaggattacctagcaagtggaggacaaccaatcaccaactgtgtgaagatgttgtgtacacacactggtacaggacaggcaattactgtaacaccagaagctaacatggaccaagagtcctttggtggtgcttcatgttgtctgtattgtagatgccacattgaccatccaaatcctaaaggattctgtgacttgaaaggtaagtacgtccaaatacctaccacttgtgctaatgacccagtgggttttacacttagaaacacagtctgtaccgtctgcggaatgtggaaaggttatggctgtagttgtgaccaactccgcgaacccttgatgcagtctgcggatgcatcaacgtttttaaacgggtttgcggtgtaagtgcagcccgtcttacaccgtgcggcacaggcactagtactgatgtcgtctacagggcttttgatatttacaacgaaaaagttgctggttttgcaaagttcctaaaaactaattgctgtcgcttccaggagaaggatgaggaaggcaatttattagactcttactttgtagttaagaggcatactatgtctaactaccaacatgaagagactatttataacttggttaaagattgtccagcggttgctgtccatgactttttcaagtttagagtagatggtgacatggtaccacatatatcacgtcagcgtctaactaaatacacaatggctgatttagtctatgctctacgtcattttgatgagggtaattgtgatacattaaaagaaatactcgtcacatacaattgctgtgatgatgattatttcaataagaaggattggtatgacttcgtagagaatcctgacatcttacgcgtatatgctaacttaggtgagcgtgtacgccaatcattattaaagactgtacaattctgcgatgctatgcgtgatgcaggcattgtaggcgtactgacattagataatcaggatcttaatgggaactggtacgatttcggtgatttcgtacaagtagcaccaggctgcggagttcctattgtggattcatattactcattgctgatgcccatcctcactttgactagggcattggctgctgagtcccatatggatgctgatctcgcaaaaccacttattaagtgggatttgctgaaatatgattttacggaagagagactttgtctcttcgaccgttattttaaatattgggaccagacataccatcccaattgtattaactgtttggatgataggtgtatccttcattgtgcaaactttaatgtgttattttctactgtgtttccacctacaagttttggaccactagtaagaaaaatatttgtagatggtgttccttttgttgtttcaactggataccattttcgtgagttaggagtcgtacataatcaggatgtaaacttacatagctcgcgtctcagtttcaaggaacttttagtgtatgctgctgatccagctatgcatgcagcttctggcaatttattgctagataaacgcactacatgcttttcagtagctgcactaacaaacaatgttgcttttcaaactgtcaaacccggtaattttaataaagacttttatgactttgctgtgtctaaaggtttctttaaggaaggaagttctgttgaactaaaacacttcttctttgctcaggatggcaacgctgctatcagtgattatgactattatcgttataatctgccaacaatgtgtgatatcagacaactcctattcgtagttgaagttgttgataaatactttgattgttacgatggtggctgtattaatgccaaccaagtaatcgttaacaatctggataaatcagctggtttcccatttaataaatggggtaaggctagactttattatgactcaatgagttatgaggatcaagatgcacttttcgcgtatactaagcgtaatgtcatccctactataactcaaatgaatcttaagtatgccattagtgcaaagaatagagctcgcaccgtagctggtgtctctatctgtagtactatgacaaatagacagtttcatcagaaattattgaagtcaatagccgccactagaggagctactgtggtaattggaacaagcaagttttacggtggctggcataatatgttaaaaactgtttacagtgatgtagaaactccacaccttatgggttgggattatccaaaatgtgacagagccatgcctaacatgcttaggataatggcctctcttgttcttgctcgcaaacataacacttgctgtaacttatcacaccgtttctacaggttagctaacgagtgtgcgcaagtattaagtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacctgccttacccagatccatcaagaatattaggcgcaggctgttttgtcgatgatattgtcaaaacagatggtacacttatgattgaaaggttcgtgtcactggctattgatgcttacccacttacaaaacatcctaatcaggagtatgctgatgtctttcacttgtatttacaatacattagaaagttacatgatgagcttactggccacatgttggacatgtattccgtaatgctaactaatgataacacctcacggtactgggaacctgagttttatgaggctatgtacacaccacatacagtcttgcaggctgtaggtgcttgtgtattgtgcaattcacagacttcacttcgttgcggtgcctgtattaggagaccattcctatgttgcaagtgctgctatgaccatgtcatttcaacatcacacaaattagtgttgtctgttaatccctatgtttgcaatgccccaggttgtgatgtcactgatgtgacacaactgtatctaggaggtatgagctattattgcaagtcacataagcctcccattagttttccattatgtgctaatggtcaggtttttggtttatacaaaaacacatgtgtaggcagtgacaatgtcactgacttcaatgcgatagcaacatgtgattggactaatgctggcgattacatacttgccaacacttgtactgagagactcaagcttttcgcagcagaaacgctcaaagccactgaggaaacatttaagctgtcatatggtattgccactgtacgcgaagtactctctgacagagaattgcatctttcatgggaggttggaaaacctagaccaccattgaacagaaactatgtctttactggttaccgtgtaactaaaaatagtaaagtacagattggagagtacacctttgaaaaaggtgactatggtgatgctgttgtgtacagaggtactacgacatacaagttgaatgttggtgattactttgtgttgacatctcacactgtaatgccacttagtgcacctactctagtgccacaagagcactatgtgagaattactggcttgtacccaacactcaacatctcagatgagttttctagcaatgttgcaaattatcaaaaggtcggcatgcaaaagtactctacactccaaggaccacctggtactggtaagagtcattttgccatcggacttgctctctattacccatctgctcgcatagtgtatacggcatgctctcatgcagctgttgatgccctatgtgaaaaggcattaaaatatttgcccatagataaatgtagtagaatcatacctgcgcgtgcgcgcgtagagtgttttgataaattcaaagtgaattcaacactagaacagtatgttttctgcactgtaaatgcattgccagaaacaactgctgacattgtagtctttgatgaaatctctatggctactaattatgacttgagtgttgtcaatgctagacttcgtgcaaaacactacgtctatattggcgatcctgctcaattaccagccccccgcacattgctgactaaaggcacactagaaccagaatattttaattcagtgtgcagacttatgaaaacaataggtccagacatgttccttggaacttgtcgccgttgtcctgctgaaattgttgacactgtgagtgctttagtttatgacaataagctaaaagcacacaaggataagtcagctcaatgcttcaaaatgttctacaaaggtgttattacacatgatgtttcatctgcaatcaacagacctcaaataggcgttgtaagagaatttcttacacgcaatcctgcttggagaaaagctgtttttatctcaccttataattcacagaacgctgtagcttcaaaaatcttaggattgcctacgcagactgttgattcatcacagggttctgaatatgactatgtcatattcacacaaactactgaaacagcacactcttgtaatgtcaaccgcttcaatgtggctatcacaagggcaaaaattggcattttgtgcataatgtctgatagagatctttatgacaaactgcaatttacaagtctagaaataccacgtcgcaatgtggctacattacaagcagaaaatgtaactggactttttaaggactgtagtaagatcattactggtcttcatcctacacaggcacctacacacctcagcgttgatataaagttcaagactgaaggattatgtgttgacataccaggcataccaaaggacatgacctaccgtagactcatctctatgatgggtttcaaaatgaattaccaagtcaatggttaccctaatatgtttatcacccgcgaagaagctattcgtcacgttcgtgcgtggattggctttgatgtagagggctgtcatgcaactagagatgctgtgggtactaacctacctctccagctaggattttctacaggtgttaacttagtagctgtaccgactggttatgttgacactgaaaataacacagaattcaccagagttaatgcaaaacctccaccaggtgaccagtttaaacatcttataccactcatgtataaaggcttgccctggaatgtagtgcgtattaagatagtacaaatgctcagtgatacactgaaaggattgtcagacagagtcgtgttcgtcctttgggcgcatggctttgagcttacatcaatgaagtactttgtcaagattggacctgaaagaacgtgttgtctgtgtgacaaacgtgcaacttgcttttctacttcatcagatacttatgcctgctggaatcattctgtgggttttgactatgtctataacccatttatgattgatgttcagcagtggggctttacgggtaaccttcagagtaaccatgaccaacattgccaggtacatggaaatgcacatgtggctagttgtgatgctatcatgactagatgtttagcagtccatgagtgctttgttaagcgcgttgattggtctgttgaataccctattataggagatgaactgagggttaattctgcttgcagaaaagtacaacacatggttgtgaagtctgcattgcttgctgataagtttccagttcttcatgacattggaaatccaaaggctatcaagtgtgtgcctcaggctgaagtagaatggaagttctacgatgctcagccatgtagtgacaaagcttacaaaatagaggaactcttctattcttatgctacacatcacgataaattcactgatggtgtttgtttgttttggaattgtaacgttgatcgttacccagccaatgcaattgtgtgtaggtttgacacaagagtcttgtcaaacttgaacttaccaggctgtgatggtggtagtttgtatgtgaataagcatgcattccacactccagctttcgataaaagtgcatttactaatttaaagcaattgcctttcttttactattctgatagtccttgtgagtctcatggcaaacaagtagtgtcggatattgattatgttccactcaaatctgctacgtgtattacacgatgcaatttaggtggtgctgtttgcagacaccatgcaaatgagtaccgacagtacttggatgcatataatatgatgatttctgctggatttagcctatggatttacaaacaatttgatacttataacctgtggaatacatttaccaggttacagagtttagaaaatgtggcttataatgttgttaataaaggacactttgatggacacgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttcagtcatggacaacttggcggtcttcatttaatgataggcttagccaagcgctcacaagattcaccacttaaattagaggattttatccctatggacagcacagtgaaaaattacttcataacagatgcgcaaacaggttcatcaaaatgtgtgtgttctgtgattgatcttttacttgatgactttgtcgagataataaagtcacaagatttgtcagtgatttcaaaagtggtcaaggttacaattgactatgctgaaatttcattcatgctttggtgtaaggatggacatgttgaaaccttctacccaaaactacaagcaagtcaagcgtggcaaccaggtgttgcgatgcctaacttgtacaagatgcaaagaatgcttcttgaaaagtgtgaccttcagaattatggtgaaaatgctgttataccaaaaggaataatgatgaatgtcgcaaagtatactcaactgtgtcaatacttaaatacacttactttagctgtaccctacaacatgagagttattcactttggtgctggctctgataaaggagttgcaccaggtacagctgtgctcagacaatggttgccaactggcacactacttgtcgattcagatcttaatgacttcgtctccgacgcagattctactttaattggagactgtgcaacagtacatacggctaataaatgggaccttattattagcgatatgtatgaccctaggaccaaacatgtgacaaaagagaatgactctaaagaagggtttttcacttatctgtgtggatttataaagcaaaaactagccctgggtggttctatagctgtaaagataacagagcattcttggaatgctgacctttacaagcttatgggccatttctcatggtggacagcttttgttacaaatgtaaatgcatcatcatcggaagcatttttaattggggctaactatcttggcaagccgaaggaacaaattgatggctataccatgcatgctaactacattttctggaggaacacaaatcctatccagttgtcttcctattcactctttgacatgagcaaatttcctcttaaattaagaggaactgctgtaatgtctcttaaggagaatcaaatcaatgatatgatttattctcttctggaaaaaggtaggcttatcattagagaaaacaacagagttgtggtttcaagtgatattcttgttaacaactaaacgaacatgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgcgcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataaacgaacttatggatttgtttatgagattttttactcttagatcaattactgcacagccagtaaaaattgacaatgcttctcctgcaagtactgttcatgctacagcaacgataccgctacaagcctcactccctttcggatggcttgttattggcgttgcatttcttgctgtttttcagagcgctaccaaaataattgcgctcaataaaagatggcagctagccctttataagggcttccagttcatttgcaatttactgctgctatttgttaccatctattcacatcttttgcttgtcgctgcaggtatggaggcgcaatttttgtacctctatgccttgatatattttctacaatgcatcaacgcatgtagaattattatgagatgttggctttgttggaagtgcaaatccaagaacccattactttatgatgccaactactttgtttgctggcacacacataactatgactactgtataccatataacagtgtcacagatacaattgtcgttactgaaggtgacggcatttcaacaccaaaactcaaagaagactaccaaattggtggttattctgaggataggcactcaggtgttaaagactatgtcgttgtacatggctatttcaccgaagtttactaccagcttgagtctacacaaattactacagacactggtattgaaaatgctacattcttcatctttaacaagcttgttaaagacccaccgaatgtgcaaatacacacaatcgacggctcttcaggagttgctaatccagcaatggatccaatttatgatgagccgacgacgactactagcgtgcctttgtaagcacaagaaagtgagtacgaacttatgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaaacgaactaactattattattattctgtttggaactttaacattgcttatcatggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacactccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaagtgacaacagatgtttcatcttgttgacttccaggttacaatagcagagatattgattatcattatgaggactttcaggattgctatttggaatcttgacgttataataagttcaatagtgagacaattatttaagcctctaactaagaagaattattcggagttagatgatgaagaacctatggagttagattatccataaaacgaacatgaaaattattctcttcctgacattgattgtatttacatcttgcgagctatatcactatcaggagtgtgttagaggtacgactgtactactaaaagaaccttgcccatcaggaacatacgagggcaattcaccatttcaccctcttgctgacaataaatttgcactaacttgcactagcacacactttgcttttgcttgtgctgacggtactcgacatacctatcagctgcgtgcaagatcagtttcaccaaaacttttcatcagacaagaggaggttcaacaagagctctactcgccactttttctcattgttgctgctctagtatttttaatactttgcttcaccattaagagaaagacagaatgaatgagctcactttaattgacttctatttgtgctttttagcctttctgctattccttgttttaataatgcttattatattttggttttcactcgaaatccaggatctagaagaaccttgtaccaaagtctaaacgaacatgaaacttctcattgttttgacttgtatttctctatgcagttgcatatgcactgtagtacagcgctgtgcatctaataaacctcatgtgcttgaagatccttgtaaggtacaacactaggggtaatacttatagcactgcttggctttgtgctctaggaaaggttttaccttttcatagatggcacactatggttcaaacatgcacacctaatgttactatcaactgtcaagatccagctggtggtgcgcttatagctaggtgttggtaccttcatgaaggtcaccaaactgctgcatttagagacgtacttgttgttttaaataaacgaacaaattaaaatgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataaacactcatgatgaccacacaaggcagatgggctatgtaaacgttttcgcaattccgtttacgatacatagtctactcttgtgcagaatgaattctcgtaactaaacagcacaagtaggtttagttaactttaatctcacatagcaatctttaatcaatgtgtaacattagggaggacttgaaagagccaccacattttcatcgaggccacgcggagtacgatcgagggtacagtgaataatgctagggagagctgcctatatggaagagccctaatgtgtaaaattaattttagtagtgctatccccatgtgattttaatagcttcttaggagaatgacaaaaaaaaaaaaaaaaaaaaaaaa

SEQ ID NO: 10 - DNA, 30119 bp, Unknown
  Middle East respiratory syndrome coronavirus, complete genome
gatttaagtgaatagcttggctatctcacttcccctcgttctcttgcagaactttgattttaacgaacttaaataaaagccctgttgtttagcgtatcgttgcacttgtctggtgggattgtggcattaatttgcctgctcatctaggcagtggacatatgctcaacactgggtataattctaattgaatactatttttcagttagagcgtcgtgtctcttgtacgtctcggtcacaatacacggtttcgtccggtgcgtggcaattcggggcacatcatgtctttcgtggctggtgtgaccgcgcaaggtgcgcgcggtacgtatcgagcagcgctcaactctgaaaaacatcaagaccatgtgtctctaactgtgccactctgtggttcaggaaacctggttgaaaaactttcaccatggttcatggatggcgaaaatgcctatgaagtggtgaaggccatgttacttaaaaaggagccacttctctatgtgcccatccggctggctggacacactagacacctcccaggtcctcgtgtgtacctggttgagaggctcattgcttgtgaaaatccattcatggttaaccaattggcttatagctctagtgcaaatggcagcctggttggcacaactttgcagggcaagcctattggtatgttcttcccttatgacatcgaacttgtcacaggaaagcaaaatattctcctgcgcaagtatggccgtggtggttatcactacaccccattccactatgagcgagacaacacctcttgccctgagtggatggacgattttgaggcggatcctaaaggcaaatatgcccagaatctgcttaagaagttgattggcggtgatgtcactccagttgaccaatacatgtgtggcgttgatggaaaacccattagtgcctacgcatttttaatggccaaggatggaataaccaaactggctgatgttgaagcggacgtcgcagcacgtgctgatgacgaaggcttcatcacattaaagaacaatctatatagattggtttggcatgttgagcgtaaagacgttccatatcctaagcaatctatttttactattaatagtgtggtccaaaaggatggtgttgaaaacactcctcctcactattttactcttggatgcaaaattttaacgctcaccccacgcaacaagtggagtggcgtttctgacttgtccctcaaacaaaaactcctttacaccttctatggtaaggagtcacttgagaacccaacctacatttaccactccgcattcattgagtgtggaagttgtggtaatgattcctggcttacagggaatgctatccaagggtttgcctgtggatgtggggcatcatatacagctaatgatgtcgaagtccaatcatctggcatgattaagccaaatgctcttctttgtgctacttgcccctttgctaagggtgatagctgttcttctaattgcaaacattcagttgctcagttggttagttacctttctgaacgctgtaatgttattgctgattctaagtccttcacacttatctttggtggcgtagcttacgcctactttggatgtgaggaaggtactatgtactttgtgcctagagctaagtctgttgtctcaaggattggagactccatctttacaggctgtactggctcttggaacaaggtcactcaaattgctaacatgttcttggaacagactcagcattcccttaactttgtgggagagttcgttgtcaacgatgttgtcctcgcaattctctctggaaccacaactaatgttgacaaaatacgccagcttctcaaaggtgtcacccttgacaagttgcgtgattatttagctgactatgacgtagcagtcactgccggcccattcatggataatgctattaatgttggtggtacaggattacagtatgccgccattactgcaccttatgtagttctcactggcttaggtgagtcctttaagaaagttgcaaccataccgtataaggtttgcaactctgttaaggatactctggcttattatgctcacagcgtgttgtacagagtttttccttatgacatggattctggtgtgtcatcctttagtgaactactttttgattgcgttgatctttcagtagcttctacctattttttagtccgcatcttgcaagataagactggcgactttatgtctacaattattacttcctgccaaactgctgttagtaagcttctagatacatgttttgaagctacagaagcaacatttaacttcttgttagatttggcaggattgttcagaatctttctccgcaatgcctatgtgtacacttcacaagggtttgtggtggtcaatggcaaagtttctacacttgtcaaacaagtgttagacttgcttaataagggtatgcaacttttgcatacaaaggtctcctgggctggttctaaaatcattgctgttatctacagcggcagggagtctctaatattcccatcgggaacctattactgtgtcaccactaaggctaagtccgttcaacaagatcttgacgttattttgcctggtgagttttccaagaagcagttaggactgctccaacctactgacaattctacaactgttagtgttactgtatccagtaacatggttgaaactgttgtgggtcaacttgagcaaactaatatgcatagtcctgatgttatagtaggtgactatgtcattattagtgaaaaattgtttgtgcgtagtaaggaagaagacggatttgccttctaccctgcttgcactaatggtcatgctgtaccgactctctttagacttaagggaggtgcacctgtaaaaaaagtagcctttggcggtgatcaagtacatgaggttgctgctgtaagaagtgttactgtcgagtacaacattcatgctgtattagacacactacttgcttcttctagtcttagaacctttgttgtagataagtctttgtcaattgaggagtttgctgacgtagtaaaggaacaagtctcagacttgcttgttaaattactgcgtggaatgccgattccagattttgatttagacgattttattgacgcaccatgctattgctttaacgctgagggtgatgcatcctggtcttctactatgatcttctctcttcaccccgtcgagtgtgacgaggagtgttctgaagtagaggcttcagatttagaagaaggtgaatcagagtgcatttctgagacttcaactgaacaagttgacgtttctcatgagacttctgacgacgagtgggctgctgcagttgatgaagcgttccctctcgatgaagcagaagatgttactgaatctgtgcaagaagaagcacaaccagtagaagtacctgttgaagatattgcgcaggttgtcatagctgacaccttacaggaaactcctgttgtgcctgatactgttgaagtcccaccgcaagtggtgaaacttccgtctgcacctcagactatccagcccgaggtaaaagaagttgcacctgtctatgaggctgataccgaacagacacagaatgttactgttaaacctaagaggttacgcaaaaagcgtaatgttgaccctttgtccaattttgaacataaggttattacagagtgcgttaccatagttttaggtgacgcaattcaagtagccaagtgctatggggagtctgtgttagttaatgctgctaacacacatcttaagcatggcggtggtatcgctggtgctattaatgcggcttcaaaaggggctgtccaaaaagagtcagatgagtatattctggctaaagggccgttacaagtaggagattcagttctcttgcaaggccattctctagctaagaatatcctgcatgtcgtaggcccagatgcccgcgctaaacaggatgtttctctccttagtaagtgctataaggctatgaatgcatatcctcttgtagtcactcctcttgtttcagcaggcatatttggtgtaaaaccagctgtgtcttttgattatcttattagggaggctaagactagagttttagtcgtcgttaattcccaagatgtctataagagtcttaccatagttgacattccacagagtttgactttttcatatgatgggttacgtggcgcaatacgtaaagctaaagattatggttttactgtttttgtgtgcacagacaactctgctaacactaaagttcttaggaacaagggtgttgattatactaagaagtttcttacagttgacggtgtgcaatattattgctacacgtctaaggacactttagatgatatcttacaacaggctaataagtctgttggtattatatctatgcctttgggatatgtgtctcatggtttagacttaatgcaagcagggagtgtcgtgcgtagagttaacgtgccctacgtgtgtctcctagctaataaagagcaagaagctattttgatgtctgaagacgttaagttaaacccttcagaagattttataaagcacgtccgcactaatggtggttacaattcttggcatttagtcgagggtgaactattggtgcaagacttacgcttaaataagctcctgcattggtctgatcaaaccatatgctacaaggatagtgtgttttatgttgtaaagaatagtacagcttttccatttgaaacactttcagcatgtcgtgcgtatttggattcacgcacgacacagcagttaacaatcgaagtcttagtgactgtcgatggtgtaaattttagaacagtcgttctaaataataagaacacttatagatcacagcttggatgcgttttctttaatggtgctgatatttctgacaccattcctgatgagaaacagaatggtcacagtttatatctagcagacaatttgactgctgatgaaacaaaggcgcttaaagagttatatggccccgttgatcctactttcttacacagattctattcacttaaggctgcagtccatgggtggaagatggttgtgtgtgataaggtacgttctctcaaattgagtgataataattgttatcttaatgcagttattatgacacttgatttattgaaggacattaaatttgttatacctgctctacagcatgcatttatgaaacataagggcggtgattcaactgacttcatagccctcattatggcttatggcaattgcacatttggtgctccagatgatgcctctcggttacttcataccgtgcttgcaaaggctgagttatgctgttctgcacgcatggtttggagagagtggtgcaatgtctgtggcataaaagatgttgttctacaaggcttaaaagcttgttgttacgtgggtgtgcaaactgttgaagatctgcgtgctcgcatgacatatgtatgccagtgtggtggtgaacgtcatcggcaattagtcgaacacaccaccccctggttgctgctctcaggcacaccaaatgaaaaattggtgacaacctccacggcgcctgattttgtagcatttaatgtctttcagggcattgaaacggctgttggccattatgttcatgctcgcctgaagggtggtcttattttaaagtttgactctggcaccgttagcaagacttcagactggaagtgcaaggtgacagatgtacttttccccggccaaaaatacagtagcgattgtaatgtcgtacggtattctttggacggtaatttcagaacagaggttgatcccgacctatctgctttctatgttaaggatggtaaatactttacaagtgaaccacccgtaacatattcaccagctacaattttagctggtagtgtctacactaatagctgccttgtatcgtctgatggacaacctggcggtgatgctattagtttgagttttaataaccttttagggtttgattctagtaaaccagtcactaagaaatacacttactccttcttgcctaaagaagacggcgatgtgttgttggctgagtttgacacttatgaccctatttataagaatggtgccatgtataaaggcaaaccaattctttgggtcaataaagcatcttatgatactaatcttaataagttcaatagagctagtttgcgtcaaatttttgacgtagcccccattgaactcgaaaataaattcacacctttgagtgtggagtctacaccagttgaacctccaactgtagatgtggtagcacttcaacaggaaatgacaattgtcaaatgtaagggtttaaataaacctttcgtgaaggacaatgtcagtttcgttgctgatgattcaggtactcccgttgttgagtatctgtctaaagaagacctacatacattgtatgtagaccctaagtatcaagtcattgtcttaaaagacaatgtactttcttctatgcttagattgcacaccgttgagtcaggtgatattaacgttgttgcagcttccggatctttgacacgtaaagtgaagttactatttagggcttcattttatttcaaagaatttgctacccgcactttcactgctaccactgctgtaggtagttgtataaagagtgtagtgcggcatctaggtgttactaaaggcatattgacaggctgttttagttttgccaagatgttatttatgcttccactagcttactttagtgattcaaaactcggcaccacagaggttaaagtgagtgctttgaaaacagccggcgttgtgacaggtaatgttgtaaaacagtgttgcactgctgctgttgatttaagtatggataagttgcgccgtgtggattggaaatcaaccctacggttgttacttatgttatgcacaactatggtattgttgtcttctgtgtatcacttgtatgtcttcaatcaggtcttatcaagtgatgttatgtttgaagatgcccaaggtttgaaaaagttctacaaagaagttagagcttacctaggaatctcttctgcttgtgacggtcttgcttcagcttatagggcgaattcctttgatgtacctacattctgcgcaaaccgttctgcaatgtgtaattggtgcttgattagccaagattccataactcactacccagctcttaagatggttcaaacacatcttagccactatgttcttaacatagattggttgtggtttgcatttgagactggtttggcatacatgctctatacctcggccttcaactggttgttgttggcaggtacattgcattatttctttgcacagacttccatatttgtagactggcggtcatacaattatgctgtgtctagtgccttctggttattcacccacattccaatggcgggtttggtacgaatgtataatttgttagcatgcctttggcttttacgcaagttttatcagcatgtaatcaatggttgcaaagatacggcatgcttgctctgctataagaggaaccgacttactagagttgaagcttctaccgttgtctgtggtggaaaacgtacgttttatatcacagcaaatggcggtatttcattctgtcgtaggcataattggaattgtgtggattgtgacactgcaggtgtggggaataccttcatctgtgaagaagtcgcaaatgacctcactaccgccctacgcaggcctattaacgctacggatagatcacattattatgtggattccgttacagttaaagagactgttgttcagtttaattatcgtagagacggtcaaccattctacgagcggtttcccctctgcgcttttacaaatctagataagttgaagttcaaagaggtctgtaaaactactactggtatacctgaatacaactttatcatctacgactcatcagatcgtggccaggaaagtttagctaggtctgcatgtgtttattattctcaagtcttgtgtaaatcaattcttttggttgactcaagtttggttacttctgttggtgattctagtgaaatcgccactaaaatgtttgattcctttgttaatagtttcgtctcgctgtataatgtcacacgcgataagttggaaaaacttatctctactgctcgtgatggcgtaaggcgaggcgataacttccatagtgtcttaacaacattcattgacgcagcacgaggccccgcaggtgtggagtctgatgttgagaccaatgaaattgttgactctgtgcagtatgctcataaacatgacatacaaattactaatgagagctacaataattatgtaccctcatatgttaaacctgatagtgtgtctaccagcgatttaggtagtctcattgattgtaatgcggcttcagttaaccaaattgtcttgcgtaattctaatggtgcttgcatttggaacgctgctgcatatatgaaactctcggatgcacttaaacgacagattcgcattgcatgccgtaagtgtaatttagctttccggttaaccacctcaaagctacgcgctaatgataatatcttatcagttagattcactgctaacaaaattgttggtggtgctcctacatggtttaatgcgttgcgtgactttacgttaaagggttatgttcttgctaccattattgtgtttctgtgtgctgtactgatgtatttgtgtttacctacattttctatggcacctgttgaattttatgaagaccgcatcttggactttaaagttcttgataatggtatcattagggatgtaaatcctgatgataagtgctttgctaataagcaccggtccttcacacaatggtatcatgagcatgttggtggtgtctatgacaactctatcacatgcccattgacagttgcagtaattgctggagttgctggtgctcgcattccagacgtacctactacattggcttgggtgaacaatcagataattttctttgtttctcgagtctttgctaatacaggcagtgtttgctacactcctatagatgagataccctataagagtttctctgatagtggttgcattcttccatctgagtgcactatgtttagggatgcagagggccgtatgacaccatactgccatgatcctactgttttgcctggggcttttgcgtacagtcagatgaggcctcatgttcgttacgacttgtatgatggtaacatgtttattaaatttcctgaagtagtatttgaaagtacacttaggattactagaactctgtcaactcagtactgccggttcggtagttgtgagtatgcacaagagggtgtttgtattaccacaaatggctcgtgggccatttttaatgaccaccatcttaatagacctggtgtctattgtggctctgattttattgacattgtcaggcggttagcagtatcactgttccagcctattacttatttccaattgactacctcattggtcttgggtataggtttgtgtgcgttcctgactttgctcttctattatattaataaagtaaaacgtgcttttgcagattacacccagtgtgctgtaattgctgttgttgctgctgttcttaatagcttgtgcatctgctttgttacctctataccattgtgtatagtaccttacactgcattgtactattatgctacattctattttactaatgagcctgcatttattatgcatgtttcttggtacattatgttcgggcctatcgttcccatatggatgacctgcgtctatacagttgcaatgtgctttagacacttcttctgggttttagcttattttagtaagaaacatgtagaagtttttactgatggtaagcttaattgtagtttccaggacgctgcctctaatatctttgttattaacaaggacacttatgcagctcttagaaactctttaactaatgatgcctattcacgatttttggggttgtttaacaagtataagtacttctctggtgctatggaaacagccgcttatcgtgaagctgcagcatgtcatcttgctaaagccttacaaacatacagcgagactggtagtgatcttctttaccaaccacccaactgtagcataacctctggcgtgttgcaaagcggtttggtgaaaatgtcacatcccagtggagatgttgaggcttgtatggttcaggttacctgcggtagcatgactcttaatggtctttggcttgacaacacagtctggtgcccacgacacgtaatgtgcccggctgaccagttgtctgatcctaattatgatgccttgttgatttctatgactaatcatagtttcagtgtgcaaaaacacattggcgctccagcaaacttgcgtgttgttggtcatgccatgcaaggcactcttttgaagttgactgtcgatgttgctaaccctagcactccagcctacacttttacaacagtgaaacctggcgcagcatttagtgtgttagcatgctataatggtcgtccgactggtacattcactgttgtaatgcgccctaactacacaattaagggttcctttctgtgtggttcttgtggtagtgttggttacaccaaggagggtagtgtgatcaatttctgttacatgcatcaaatggaacttgctaatggtacacataccggttcagcatttgatggtactatgtatggtgcctttatggataaacaagtgcaccaagttcagttaacagacaaatactgcagtgttaatgtagtagcttggctttacgcagcaatacttaatggttgcgcttggtttgtaaaacctaatcgcactagtgttgtttcttttaatgaatgggctcttgccaaccaattcactgaatttgttggcactcaatccgttgacatgttagctgtcaaaacaggcgttgctattgaacagctgctttatgcgatccaacaactgtatactgggttccagggaaagcaaatccttggcagtaccatgttggaagatgaattcacacctgaggatgttaatatgcagattatgggtgtggttatgcagagtggtgtgagaaaagttacatatggtactgcgcattggttgtttgcgacccttgtctcaacctatgtgataatcttacaagccactaaatttactttgtggaactacttgtttgagactattcccacacagttgttcccactcttatttgtgactatggccttcgttatgttgttggttaaacacaaacacacctttttgacacttttcttgttgcctgtggctatttgtttgacttatgcaaacatagtctacgagcccactactcccatttcgtcagcgctgattgcagttgcaaattggcttgcccccactaatgcttatatgcgcactacacatactgatattggtgtctacattagtatgtcacttgtattagtcattgtagtgaagagattgtacaacccatcactttctaactttgcgttagcattgtgcagtggtgtaatgtggttgtacacttatagcattggagaagcctcaagccccattgcctatctggtttttgtcactacactcactagtgattatacgattacagtctttgttactgtcaaccttgcaaaagtttgcacttatgccatctttgcttactcaccacagcttacacttgtgtttccggaagtgaagatgatacttttattatacacatgtttaggtttcatgtgtacttgctattttggtgtcttctctcttttgaaccttaagcttagagcacctatgggtgtctatgactttaaggtctcaacacaagagttcagattcatgactgctaacaatctaactgcacctagaaattcttgggaggctatggctctgaactttaagttaataggtattggcggtacaccttgtataaaggttgctgctatgcagtctaaacttacagatcttaaatgcacatctgtggttctcctctctgtgctccaacagttacacttagaggctaatagtagggcctgggctttctgtgttaaatgccataatgatatattggcagcaacagaccccagtgaggctttcgagaaattcgtaagtctctttgctactttaatgactttttctggtaatgtagatcttgatgcgttagctagtgatatttttgacactcctagcgtacttcaagctactctttctgagttttcacacttagctacctttgctgagttggaagctgcgcagaaagcctatcaggaagctatggactctggtgacacctcaccacaagttcttaaggctttgcagaaggctgttaatatagctaaaaacgcctatgagaaggataaggcagtggcccgtaagttagaacgtatggctgatcaggctatgacttctatgtataagcaagcacgtgctgaagacaagaaagcaaaaattgtcagtgctatgcaaactatgttgtttggtatgattaagaagctcgacaacgatgttcttaatggtatcatttctaacgctaggaatggttgtatacctcttagtgtcatcccactgtgtgcttcaaataaacttcgcgttgtaattcctgacttcaccgtctggaatcaggtagtcacatatccctcgcttaactacgctggggctttgtgggacattacagttataaacaatgtggacaatgaaattgttaagtcttcagatgttgtagacagcaatgaaaatttaacatggccacttgttttagaatgcactagggcatccacttctgccgttaagttgcaaaataatgagatcaaaccttcaggtctaaaaaccatggttgtgtctgcgggtcaagagcaaactaactgtaatactagttccttagcttattacgaacctgtgcagggtcgtaaaatgctgatggctcttctttctgataatgcctatctcaaatgggcgcgtgttgaaggtaaggacggatttgtcagtgtagagctacaacctccttgcaaattcttgattgcgggaccaaaaggacctgaaatccgatatctctattttgttaaaaatcttaacaaccttcatcgcgggcaagtgttagggcacattgctgcgactgttagattgcaagctggttctaacaccgagtttgcctctaattcctcggtgttgtcacttgttaacttcaccgttgatcctcaaaaagcttatctcgatttcgtcaatgcgggaggtgccccattgacaaattgtgttaagatgcttactcctaaaactggtacaggtatagctatatctgttaaaccagagagtacagctgatcaagagacttatggtggagcttcagtgtgtctctattgccgtgcgcatatagaacatcctgatgtctctggtgtttgtaaatataagggtaagtttgtccaaatccctgctcagtgtgtccgtgaccctgtgggattttgtttgtcaaataccccctgtaatgtctgtcaatattggattggatatgggtgcaattgtgactcgcttaggcaagcagcactgccccaatctaaagattccaattttttaaacgagtccggggttctattgtaaatgcccgaatagaaccctgttcaagtggtttgtccactgatgtcgtctttagggcatttgacatctgcaactataaggctaaggttgctggtattggaaaatactacaagactaatacttgtaggtttgtagaattagatgaccaagggcatcatttagactcctattttgtcgttaagaggcatactatggagaattatgaactagagaagcactgttacgacttgttacgtgactgtgatgctgtagctccccatgatttcttcatctttgatgtagacaaagttaaaacacctcatattgtacgtcagcgtttaactgagtacactatgatggatcttgtatatgccctgaggcactttgatcaaaatagcgaagtgcttaaggctatcttagtgaagtatggttgctgtgatgttacctactttgaaaataaactctggtttgattttgttgaaaatcccagtgttattggtgtttatcataaacttggagaacgtgtacgccaagctatcttaaacactgttaaattttgtgaccacatggtcaaggctggtttagtcggtgtgctcacactagacaaccaggaccttaatggcaagtggtatgattttggtgacttcgtaatcactcaacctggttcaggagtagctatagttgatagctactattcttatttgatgcctgtgctctcaatgaccgattgtctggccgctgagacacatagggattgtgattttaataaaccactcattgagtggccacttactgagtatgattttactgattataaggtacaactctttgagaagtactttaaatattgggatcagacgtatcacgcaaattgcgttaattgtactgatgaccgttgtgtgttacattgtgctaatttcaatgtattgtttgctatgaccatgcctaagacttgtttcggacccatagtccgaaagatctttgttgatggcgtgccatttgtagtatcttgtggttatcactacaaagaattaggtttagtcatgaatatggatgttagtctccatagacataggctctctcttaaggagttgatgatgtatgccgctgatccagccatgcacattgcctcctctaacgcttttcttgatttgaggacatcatgttttagtgtcgctgcacttacaactggtttgacttttcaaactgtgcggcctggcaattttaaccaagacttctatgatttcgtggtatctaaaggtttctttaaggagggctcttcagtgacgctcaaacattttttctttgctcaagatggtaatgctgctattacagattataattactattcttataatctgcctactatgtgtgacatcaaacaaatgttgttctgcatggaagttgtaaacaagtacttcgaaatctatgacggtggttgtcttaatgcttctgaagtggttgttaataatttagacaagagtgctggccatccttttaataagtttggcaaagctcgtgtctattatgagagcatgtcttaccaggagcaagatgaactttttgccatgacaaagcgtaacgtcattcctaccatgactcaaatgaatctaaaatatgctattagtgctaagaatagagctcgcactgttgcaggcgtgtccatacttagcacaatgactaatcgccagtaccatcagaaaatgcttaagtccatggctgcaactcgtggagcgacttgcgtcattggtactacaaagttctacggtggctgggatttcatgcttaaaacattgtacaaagatgttgataatccgcatcttatgggttgggattaccctaagtgtgatagagctatgcctaatatgtgtagaatcttcgcttcactcatattagctcgtaaacatggcacttgttgtactacaagggacagattttatcgcttggcaaatgagtgtgctcaggtgctaagcgaatatgttctatgtggtggtggttactacgtcaaacctggaggtaccagtagcggagatgccaccactgcatatgccaatagtgtctttaacattttgcaggcgacaactgctaatgtcagtgcacttatgggtgctaatggcaacaagattgttgacaaagaagttaaagacatgcagtttgatttgtatgtcaatgtttacaggagcactagcccagaccccaaatttgttgataaatactatgcttttcttaataagcacttttctatgatgatactgtctgatgacggtgtcgtttgctataatagtgattatgcagctaagggttacattgctggaatacagaattttaaggaaacgctgtattatcagaacaatgtctttatgtctgaagctaaatgctgggtggaaaccgatctgaagaaagggccacatgaattctgttcacagcatacgctttatattaaggatggcgacgatggttacttccttccttatccagacccttcaagaattttgtctgccggttgctttgtagatgatatcgttaagactgacggtacactcatggtagagcggtttgtgtctttggctatagatgcttaccctctcacaaagcatgaagatatagaataccagaatgtattctgggtctacttacagtatatagaaaaactgtataaagaccttacaggacacatgcttgacagttattctgtcatgctatgtggtgataattctgctaagttttgggaagaggcattctatagagatctctatagttcgcctaccactttgcaggctgtcggttcatgcgttgtatgccattcacagacttccctacgctgtgggacatgcatccgtagaccatttctctgctgtaaatgctgctatgatcatgttatagcaactccacataagatggttttgtctgtttctccttacgtttgtaatgcccctggttgtggcgtttcagacgttactaagctatatttaggtggtatgagctacttttgtgtagatcatagacctgtgtgtagttttccactttgcgctaatggtcttgtattcggcttatacaagaatatgtgcacaggtagtccttctatagttgaatttaataggttggctacctgtgactggactgaaagtggtgattacacccttgccaatactacaacagaaccactcaaactttttgctgctgagactttacgtgccactgaagaggcgtctaagcagtcttatgctattgccaccatcaaagaaattgttggtgagcgccaactattacttgtgtgggaggctggcaagtccaaaccaccactcaatcgtaattatgtttttactggttatcatataaccaaaaatagtaaagtgcagctcggtgagtacattttcgagcgcattgattatagtgatgctgtatcctacaagtctagtacaacgtataaactgactgtaggtgacatcttcgtacttacctctcactctgtggctaccttgacggcgcccacaattgtgaatcaagagaggtatgttaaaattactgggttgtacccaaccattacggtacctgaagagttcgcaagtcatgttgccaacttccaaaaatcaggttatagtaaatatgtcactgttcagggaccacctggcactggcaaaagtcattttgctatagggttagcgatttactaccctacagcacgtgttgtttatacagcatgttcacacgcagctgttgatgctttgtgtgaaaaagcttttaaatatttgaacattgctaaatgttcccgtatcattcctgcaaaggcacgtgttgagtgctatgacaggtttaaagttaatgagacaaattctcaatatttgtttagtactattaatgctctaccagaaacttctgccgatattctggtggttgatgaggttagtatgtgcactaattatgatctttcaattattaatgcacgtattaaagctaagcacattgtctatgtaggagatccagcacagttgccagctcctaggactttgttgactagaggcacattggaaccagaaaatttcaatagtgtcactagattgatgtgtaacttaggtcctgacatatttttaagtatgtgctacaggtgtcctaaggaaatagtaagcactgtgagcgctcttgtctacaataataaattgttagccaagaaggagctttcaggccagtgctttaaaatactctataagggcaatgtgacgcatgatgctagctctgccattaatagaccacaactcacatttgtgaagaattttattactgccaatccggcatggagtaaggcagtctttatttcgccttacaattcacagaatgctgtgtctcgttcaatgctgggtcttaccactcagactgttgattcctcacagggttcagaataccagtacgttatcttctgtcaaacagcagatacggcacatgctaacaacattaacagatttaatgttgcaatcactcgtgcccaaaaaggtattctttgtgttatgacatctcaggcactctttgagtccttagagtttactgaattgtcttttactaattacaagctccagtctcagattgtaactggcctttttaaagattgctctagagaaacttctggcctctcacctgcttatgcaccaacatatgttagtgttgatgacaagtataagacgagtgatgagctttgcgtgaatcttaatttacccgcaaatgtcccatactctcgtgttatttccaggatgggctttaaactcgatgcaacagttcctggatatcctaagcttttcattactcgtgaagaggctgtaaggcaagttcgaagctggataggcttcgatgttgagggtgctcatgcttcccgtaatgcatgtggcaccaatgtgcctctacaattaggattttcaactggtgtgaactttgttgttcagccagttggtgttgtagacactgagtggggtaacatgttaacgggcattgctgcacgtcctccaccaggtgaacagtttaagcacctcgtgcctcttatgcataagggggctgcgtggcctattgttagacgacgtatagtgcaaatgttgtcagacactttagacaaattgtctgattactgtacgtttgtttgttgggctcatggctttgaattaacgtctgcatcatacttttgcaagataggtaaggaacagaagtgttgcatgtgcaatagacgcgctgcagcgtactcttcacctctgcaatcttatgcctgctggactcattcctgcggttatgattatgtctacaaccctttctttgtcgatgttcaacagtggggttatgtaggcaatcttgctactaatcacgatcgttattgctctgtccatcaaggagctcatgtggcttctaatgatgcaataatgactcgttgtttagctattcattcttgttttatagaacgtgtggattgggatatagagtatccttatatctcacatgaaaagaaattgaattcctgttgtagaatcgttgagcgcaacgtcgtacgtgctgctcttcttgccggttcatttgacaaagtctatgatattggcaatcctaaaggaattcctattgttgatgaccctgtggttgattggcattattttgatgcacagcccttgaccaggaaggtacaacagcttttctatacagaggacatggcctcaagatttgctgatgggctctgcttattttggaactgtaatgtaccaaaatatcctaataatgcaattgtatgcaggtttgacacacgtgtgcattctgagttcaatttgccaggttgtgatggcggtagtttgtatgttaacaagcacgcttttcatacaccagcatatgatgtgagtgcattccgtgatctgaaacctttaccattcttttattattctactacaccatgtgaagtgcatggtaatggtagtatgatagaggatattgattatgtacccctaaaatctgcagtctgtattacagcttgtaatttagggggcgctgtttgtaggaagcatgctacagagtacagagagtatatggaagcatataatcttgtctctgcatcaggtttccgcctttggtgttataagacctttgatatttataatctctggtctacttttacaaaagttcaaggtttggaaaacattgcttttaatgttgttaaacaaggccattttattggtgttgagggtgaactacctgtagctgtagtcaatgataagatcttcaccaagagtggcgttaatgacatttgtatgtttgagaataaaaccactttgcctactaatatagcttttgaactctatgctaagcgtgctgtacgctcgcatcccgatttcaaattgctacacaatttacaagcagacatttgctacaagttcgtcctttgggattatgaacgtagcaatatttatggtactgctactattggtgtatgtaagtacactgatattgatgttaattcagctttgaatatatgttttgacatacgcgataattgttcattggagaagttcatgtctactcccaatgccatctttatttctgatagaaaaatcaagaaatacccttgtatggtaggtcctgattatgcttacttcaatggtgctatcatccgtgatagtgatgttgttaaacaaccagtgaagttctacttgtataagaaagtcaataatgagtttattgatcctactgagtgtatttacactcagagtcgctcttgtagtgacttcctacccctttctgacatggagaaagactttctatcttttgatagtgatgttttcattaagaagtatggcttggaaaactatgcttttgagcacgtagtctatggagacttctctcatactacgttaggcggtcttcacttgcttattggtttatacaagaagcaacaggaaggtcatattattatggaagaaatgctaaaaggtagctcaactattcataactattttattactgagactaacacagcggcttttaaggcggtgtgttctgttatagatttaaagcttgacgactttgttatgattttaaagagtcaagaccttggcgtagtatccaaggttgtcaaggttcctattgacttaacaatgattgagtttatgttatggtgtaaggatggacaggttcaaaccttctaccctcgactccaggcttctgcagattggaaacctggtcatgcaatgccatccctctttaaagttcaaaatgtaaaccttgaacgttgtgagcttgctaattacaagcaatctattcctatgcctcgcggtgtgcacatgaacatcgctaaatatatgcaattgtgccagtatttaaatacttgcacattagccgtgcctgccaatatgcgtgttatacattttggcgctggttctgataaaggtatcgctcctggtacctcagttttacgacagtggcttcctacagatgccattattatagataatgatttaaatgagttcgtgtcagatgctgacataactttatttggagattgtgtaactgtacgtgtcggccaacaagtggatcttgttatttccgacatgtatgatcctactactaagaatgtaacaggtagtaatgagtcaaaggctttattctttacttacctgtgtaacctcattaataataatcttgctcttggtgggtctgttgctattaaaataacagaacactcttggagcgttgaactttatgaacttatgggaaaatttgcttggtggactgttttctgcaccaatgcaaatgcatcctcatctgaaggattcctcttaggtattaattacttgggtactattaaagaaaatatagatggtggtgctatgcacgccaactatatattttggagaaattccactcctatgaatctgagtacttactcactttttgatttatccaagtttcaattaaaattaaaaggaacaccagttcttcaattaaaggagagtcaaattaacgaactcgtaatatctctcctgtcgcagggtaagttacttatccgtgacaatgatacactcagtgtttctactgatgttcttgttaacacctacagaaagttacgttgatgtagggccagattctgttaagtctgcttgtattgaggttgatatacaacagactttctttgataaaacttggcctaggccaattgatgtttctaaggctgacggtattatataccctcaaggccgtacatattctaacataactatcacttatcaaggtctttttccctatcagggagaccatggtgatatgtatgtttactctgcaggacatgctacaggcacaactccacaaaagttgtttgtagctaactattctcaggacgtcaaacagtttgctaatgggtttgtcgtccgtataggagcagctgccaattccactggcactgttattattagcccatctaccagcgctactatacgaaaaatttaccctgcttttatgctgggttcttcagttggtaatttctcagatggtaaaatgggccgcttcttcaatcatactctagttcttttgcccgatggatgtggcactttacttagagctttttattgtattctagagcctcgctctggaaatcattgtcctgctggcaattcctatacttcttttgccacttatcacactcctgcaacagattgttctgatggcaattacaatcgtaatgccagtctgaactcttttaaggagtattttaatttacgtaactgcacctttatgtacacttataacattaccgaagatgagattttagagtggtttggcattacacaaactgctcaaggtgttcacctcttctcatctcggtatgttgatttgtacggcggcaatatgtttcaatttgccaccttgcctgtttatgatactattaagtattattctatcattcctcacagtattcgttctatccaaagtgatagaaaagcttgggctgccttctacgtatataaacttcaaccgttaactttcctgttggatttttctgttgatggttatatacgcagagctatagactgtggttttaatgatttgtcacaactccactgctcatatgaatccttcgatgttgaatctggagtttattcagtttcgtctttcgaagcaaaaccttctggctcagttgtggaacaggctgaaggtgttgaatgtgatttttcacctcttctgtctggcacacctcctcaggtttataatttcaagcgtttggtttttaccaattgcaattataatcttaccaaattgctttcacttttttctgtgaatgattttacttgtagtcaaatatctccagcagcaattgctagcaactgttattcttcactgattttggattacttttcatacccacttagtatgaaatccgatctcagtgttagttctgctggtccaatatcccagtttaattataaacagtccttttctaatcccacatgtttgattttagcgactgttcctcataaccttactactattactaagcctcttaagtacagctatattaacaagtgctctcgtcttctttctgatgatcgtactgaagtacctcagttagtgaacgctaatcaatactcaccctgtgtatccattgtcccatccactgtgtgggaagacggtgattattataggaaacaactatctccacttgaaggtggtggctggcttgttgctagtggctcaactgttgccatgactgagcaattacagatgggctttggtattacagttcaatatggtacagacaccaatagtgtttgccccaagcttgaatttgctaatgacacaaaaattgcctctcaattaggcaattgcgtggaatattccctctatggtgtttcgggccgtggtgtttttcagaattgcacagctgtaggtgttcgacagcagcgctttgtttatgatgcgtaccagaatttagttggctattattctgatgatggcaactactactgtttgcgtgcttgtgttagtgttcctgtttctgtcatctatgataaagaaactaaaacccacgctactctatttggtagtgttgcatgtgaacacatttcttctaccatgtctcaatactcccgttctacgcgatcaatgcttaaacggcgagattctacatatggcccccttcagacacctgttggttgtgtcctaggacttgttaattcctctttgttcgtagaggactgcaagttgcctcttggtcaatctctctgtgctcttcctgacacacctagtactctcacacctcgcagtgtgcgctctgttccaggtgaaatgcgcttggcatccattgcttttaatcatcctattcaggttgatcaacttaatagtagttattttaaattaagtatacccactaatttttcctttggtgtgactcaggagtacattcagacaaccattcagaaagttactgttgattgtaaacagtacgtttgcaatggtttccagaagtgtgagcaattactgcgcgagtatggccagttttgttccaaaataaaccaggctctccatggtgccaatttacgccaggatgattctgtacgtaatttgtttgcgagcgtgaaaagctctcaatcatctcctatcataccaggttttggaggtgactttaatttgacacttctagaacctgtttctatatctactggcagtcgtagtgcacgtagtgctattgaggatttgctatttgacaaagtcactatagctgatcctggttatatgcaaggttacgatgattgcatgcagcaaggtccagcatcagctcgtgatcttatttgtgctcaatatgtggctggttacaaagtattacctcctcttatggatgttaatatggaagccgcgtatacttcatctttgcttggcagcatagcaggtgttggctggactgctggcttatcctcctttgctgctattccatttgcacagagtatcttttataggttaaacggtgttggcattactcaacaggttctttcagagaaccaaaagcttattgccaataagtttaatcaggctctgggagctatgcaaacaggcttcactacaactaatgaagcttttcagaaggttcaggatgctgtgaacaacaatgcacaggctctatccaaattagctagcgagctatctaatacttttggtgctatttccgcctctattggagacatcatacaacgtcttgatgttctcgaacaggacgcccaaatagacagacttattaatggccgtttgacaacactaaatgcttttgttgcacagcagcttgttcgttccgaatcagctgctctttccgctcaattggctaaagataaagtcaatgagtgtgtcaaggcacaatccaagcgttctggattttgcggtcaaggcacacatatagtgtcctttgttgtaaatgcccctaatggcctttacttcatgcatgttggttattaccctagcaaccacattgaggttgtttctgcttatggtctttgcgatgcagctaaccctactaattgtatagcccctgttaatggctactttattaaaactaataacactaggattgttgatgagtggtcatatactggctcgtccttctatgcacctgagcccattacctcccttaatactaagtatgttgcaccacaggtgacataccaaaacatttctactaacctccctcctcctcttctcggcaattccaccgggattgacttccaagatgagttggatgagtttttcaaaaatgttagcaccagtatacctaattttggttccctaacacagattaatactacattactcgatcttacctacgagatgttgtctcttcaacaagttgttaaagcccttaatgagtcttacatagaccttaaagagcttggcaattatacttattacaacaaatggccgtggtacatttggcttggtttcattgctgggcttgttgccttagctctatgcgtcttcttcatactgtgctgcactggttgtggcacaaactgtatgggaaaacttaagtgtaatcgttgttgtgatagatacgaggaatacgacctcgagccgcataaggttcatgttcactaattaacgaactattaatgagagttcaaagaccacccactctcttgttagtgttttcactctctcttttggtcactgcatcctcaaaacctctctatgtacctgagcattgtcagaattattctggttgcatgcttagggcttgtattaaaactgcccaagctgatacagctggtctttatacaaattttcgaattgacgtcccatctgcagaatcaactggtactcaatcagtttctgtcgatcttgagtcaacttcaactcatgatggtcctaccgaacatgttactagtgtgaatctttttgacgttggttactcagttaattaacgaactctatggattacgtgtctctgcttaatcaaatttggcagaagtaccttaactcaccgtatactacttgtttgtacatccctaaacccacagctaagtatacacctttagttggcacttcattgcaccctgtgctgtggaactgtcagctatcctttgctggttatactgaatctgctgttaattctacaaaagctttggccaaacaggacgcagctcagcgaatcgcttggttgctacataaggatggaggaatccctgatggatgttccctctacctccggcactcaagtttattcgcgcaaagcgaggaagaggagccattctccaactaagaaactgcgctacgttaagcgtagattttctcttctgcgccatgaagaccttagtgttattgtccaaccaacacactatgtcagggttacattttcagaccccaacatgtggtatctacgttcgggtcatcatttacactcagttcacaattggcttaaaccttatggcggccaacctgtttctgagtaccatattactctagctttgctaaatctcactgatgaagatttagctagagatttttcacccattgcgctctttttgcgcaatgtcagatttgagctacatgagttcgccttgctgcgcaaaactcttgttcttaatgcatcagagatctactgtgctaacatacatagatttaagcctgtgtatagagttaacacggcaatccctactattaaggattggcttctcgttcagggattttccctttaccatagtggcctccctttacatatgtcaatctctaaattgcatgcactggatgatgttactcgcaattacatcattacaatgccatgctttagaacttaccctcaacaaatgtttgttactcctttggccgtagatgttgtctccatacggtcttccaatcagggtaataaacaaattgttcattcttatcccattttacatcatccaggattttaacgaactatggctttctcggcgtctttatttaaacccgtccagctagtcccagtttctcctgcatttcatcgcattgagtctactgactctattgttttcacatacattcctgctagcggctatgtagctgctttagctgtcaatgtgtgtctcattcccctattattactgctacgtcaagatacttgtcgtcgcagcattatcagaactatggttctctatttccttgttctgtataactttttattagccattgtactagtcaatggtgtacattatccaactggaagttgcctgatagccttcttagttatcctcataatactttggtttgtagatagaattcgtttctgtctcatgctgaattcctacattccactgtttgacatgcgttcccactttattcgtgttagtacagtttcttctcatggtatggtccctgtaatacacaccaaaccattatttattagaaacttcgatcagcgttgcagctgttctcgttgtttttatttgcactcttccacttatatagagtgcacttatattagccgttttagtaagattagcctagtttctgtaactgacttctccttaaacggcaatgtttccactgttttcgtgcctgcaacgcgcgattcagttcctcttcacataatcgccccgagctcgcttatcgtttaagcagctctgcgctactatgggtcccgtgtagaggctaatccattagtctctctttggacatatggaaaacgaactatgttaccctttgtccaagaacgaatagggttgttcatagtaaactttttcatttttaccgtagtatgtgctataacactcttggtgtgtatggctttccttacggctactagattatgtgtgcaatgtatgacaggcttcaataccctgttagttcagcccgcattatacttgtataatactggacgttcagtctatgtaaaattccaggatagtaaaccccctctaccacctgacgagtgggtttaacgaactccttcataatgtctaatatgacgcaactcactgaggcgcagattattgccattattaaagactggaactttgcatggtccctgatctttctcttaattactatcgtactacagtatggatacccatcccgtagtatgactgtctatgtctttaaaatgtttgttttatggctcctatggccatcttccatggcgctatcaatatttagcgccgtttatccaattgatctagcttcccagataatctctggcattgtagcagctgtttcagctatgatgtggatttcctactttgtgcagagtatccggctgtttatgagaactggatcatggtggtcattcaatcctgagactaattgccttttgaacgttccatttggtggtacaactgtcgtacgtccactcgtagaggactctaccagtgtaactgctgttgtaaccaatggccacctcaaaatggctggcatgcatttcggtgcttgtgactacgacagacttcctaatgaagtcaccgtggccaaacccaatgtgctgattgctttaaaaatggtgaagcggcaaagctacggaactaattccggcgttgccatttaccatagatataaggcaggtaattacaggagtccgcctattacggcggatattgaacttgcattgcttcgagcttaggctctttagtaagagtatcttaattgattttaacgaatctcaatttcattgttatggcatcccctgctgcacctcgtgctgtttcctttgccgataacaatgatataacaaatacaaacctatctcgaggtagaggacgtaatccaaaaccacgagctgcaccaaataacactgtctcttggtacactgggcttacccaacacgggaaagtccctcttacctttccacctgggcagggtgtacctcttaatgccaattctacccctgcgcaaaatgctgggtattggcggagacaggacagaaaaattaataccgggaatggaattaagcaactggctcccaggtggtacttctactacactggaactggacccgaagcagcactcccattccgggctgttaaggatggcatcgtttgggtccatgaagatggcgccactgatgctccttcaacttttgggacgcggaaccctaacaatgattcagctattgttacacaattcgcgcccggtactaagcttcctaaaaacttccacattgaggggactggaggcaatagtcaatcatcttcaagagcctctagcttaagcagaaactcttccagatctagttcacaaggttcaagatcaggaaactctacccgcggcacttctccaggtccatctggaatcggagcagtaggaggtgatctactttaccttgatcttctgaacagactacaagcccttgagtctggcaaagtaaagcaatcgcagccaaaagtaatcactaagaaagatgctgctgctgctaaaaataagatgcgccacaagcgcacttccaccaaaagtttcaacatggtgcaagcttttggtcttcgcggaccaggagacctccagggaaactttggtgatcttcaattgaataaactcggcactgaggacccacgttggccccaaattgctgagcttgctcctacagccagtgcttttatgggtatgtcgcaatttaaacttacccatcagaacaatgatgatcatggcaaccctgtgtacttccttcggtacagtggagccattaaacttgacccaaagaatcccaactacaataagtggttggagcttcttgagcaaaatattgatgcctacaaaaccttccctaagaaggaaaagaaacaaaaggcaccaaaagaagaatcaacagaccaaatgtctgaacctccaaaggagcagcgtgtgcaaggtagcatcactcagcgcactcgcacccgtccaagtgttcagcctggtccaatgattgatgttaacactgattagtgtcactcaaagtaacaagatcgcggcaatcgtttgtgtttggcaaccccatctcaccatcgcttgtccactcttgcacagaatggaatcatgttgtaattacagtgcaataaggtaattataacccatttaattgatagctatgctttattaaagtgtgtagctgtagagagaatgttaaagactgtcacctctgcttgattgcaagtgaacagtgccccccgggaagagctctacagtgtgaaatgtaaataaaaaatagctattattcaattagattaggctaattagatgatttgcaaaaaaaaaaaa

SEQ ID NO: 11 - DNA, 18959 bp, Ebola virus
cggacacacaaaaagaaagaagaatttttaggatcttttgtgtgcgaataactatgaggaagattaataattttcctctcattgaaatttatatcggaatttaaattgaaattgttactgtaatcacacctggtttgtttcagagccacatcacaaagatagagaacaacctaggtctccgaagggagcaagggcatcagtgtgctcagttgaaaatcccttgtcaacacctaggtcttatcacatcacaagttccacctcagactctgcagggtgatccaacaaccttaatagaaacattattgttaaaggacagcattagttcacagtcaaacaagcaagattgagaattaaccttggttttgaacttgaacacttaggggattgaagattcaacaaccctaaagcttggggtaaaacattggaaatagttaaaagacaaattgctcggaatcacaaaattccgagtatggattctcgtcctcagaaaatctggatggcgccgagtctcactgaatctgacatggattaccacaagatcttgacagcaggtctgtccgttcaacaggggattgttcggcaaagagtcatcccagtgtatcaagtaaacaatcttgaagaaatttgccaacttatcatacaggcctttgaagcaggtgttgattttcaagagagtgcggacagtttccttctcatgctttgtcttcatcatgcgtaccagggagattacaaacttttcttggaaagtggcgcagtcaagtatttggaagggcacgggttccgttttgaagtcaagaagcgtgatggagtgaagcgccttgaggaattgctgccagcagtatctagtggaaaaaacattaagagaacacttgctgccatgccggaagaggagacaactgaagctaatgccggtcagtttctctcctttgcaagtctattccttccgaaattggtagtaggagaaaaggcttgccttgagaaggttcaaaggcaaattcaagtacatgcagagcaaggactgatacaatatccaacagcttggcaatcagtaggacacatgatggtgattttccgtttgatgcgaacaaattttctgatcaaatttctcctaatacaccaagggatgcacatggttgccgggcatgatgccaacgatgctgtgatttcaaattcagtggctcaagctcgtttttcaggcttattgattgtcaaaacagtacttgatcatatcctacaaaagacagaacgaggagttcgtctccatcctcttgcaaggaccgccaaggtaaaaaatgaggtgaactcctttaaggctgcactcagctccctggccaagcatggagagtatgctcctttcgcccgacttttgaacctttctggagtaaataatcttgagcatggtcttttccctcaactatcggcaattgcactcggagtcgccacagcacacgggagtaccctcgcaggagtaaatgttggagaacagtatcaacaactcagagaggctgccactgaggctgagaagcaactccaacaatatgcagagtctcgcgaacttgaccatcttggacttgatgatcaggaaaagaaaattcttatgaacttccatcagaaaaagaacgaaatcagcttccagcaaacaaacgctatggtaactctaagaaaagagcgcctggccaagctgacagaagctatcactgctgcgtcactgcccaaaacaagtggacattacgatgatgatgacgacattccctttccaggacccatcaatgatgacgacaatcctggccatcaagatgatgatccgactgactcacaggatacgaccattcccgatgtggtggttgatcccgatgatggaagctacggcgaataccagagttactcggaaaacggcatgaatgcaccagatgacttggtcctattcgatctagacgaggacgacgaggacactaagccagtgcctaatagatcgaccaagggtggacaacagaagaacagtcaaaagggccagcatatagagggcagacagacacaatccaggccaattcaaaatgtcccaggccctcacagaacaatccaccacgccagtgcgccactcacggacaatgacagaagaaatgaaccctccggctcaaccagccctcgcatgctgacaccaattaacgaagaggcagacccactggacgatgccgacgacgagacgtctagccttccgcccttggagtcagatgatgaagagcaggacagggacggaacttccaaccgcacacccactgtcgccccaccggctcccgtatacagagatcactctgaaaagaaagaactcccgcaagacgagcaacaagatcaggaccacactcaagaggccaggaaccaggacagtgacaacacccagtcagaacactcttttgaggagatgtatcgccacattctaagatcacaggggccatttgatgctgttttgtattatcatatgatgaaggatgagcctgtagttttcagtaccagtgatggcaaagagtacacgtatccagactcccttgaagaggaatatccaccatggctcactgaaaaagaggctatgaatgaagagaatagatttgttacattggatggtcaacaattttattggccggtgatgaatcacaagaataaattcatggcaatcctgcaacatcatcagtgaatgagcatggaacaatgggatgattcaaccgacaaatagctaacattaagtagtcaaggaacgaaaacaggaagaatttttgatgtctaaggtgtgaattattatcacaataaaagtgattcttatttttgaatttaaagctagcttattattactagccgtttttcaaagttcaatttgagtcttaatgcaaataggcgttaagccacagttatagccataattgtaactcaatattctaactagcgatttatctaaattaaattacattatgcttttataacttacctactagcctgcccaacatttacacgatcgttttataattaagaaaaaactaatgatgaagattaaaaccttcatcatccttacgtcaattgaattctctagcactcgaagcttattgtcttcaatgtaaaagaaaagctggtctaacaagatgacaactagaacaaagggcaggggccatactgcggccacgactcaaaacgacagaatgccaggccctgagctttcgggctggatctctgagcagctaatgaccggaagaattcctgtaagcgacatcttctgtgatattgagaacaatccaggattatgctacgcatcccaaatgcaacaaacgaagccaaacccgaagacgcgcaacagtcaaacccaaacggacccaatttgcaatcatagttttgaggaggtagtacaaacattggcttcattggctactgttgtgcaacaacaaaccatcgcatcagaatcattagaacaacgcattacgagtcttgagaatggtctaaagccagtttatgatatggcaaaaacaatctcctcattgaacagggtttgtgctgagatggttgcaaaatatgatcttctggtgatgacaaccggtcgggcaacagcaaccgctgcggcaactgaggcttattgggccgaacatggtcaaccaccacctggaccatcactttatgaagaaagtgcgattcggggtaagattgaatctagagatgagaccgtccctcaaagtgttagggaggcattcaacaatctaaacagtaccacttcactaactgaggaaaattttgggaaacctgacatttcggcaaaggatttgagaaacattatgtatgatcacttgcctggttttggaactgctttccaccaattagtacaagtgatttgtaaattgggaaaagatagcaactcattggacatcattcatgctgagttccaggccagcctggctgaaggagactctcctcaatgtgccctaattcaaattacaaaaagagttccaatcttccaagatgctgctccacctgtcatccacatccgctctcgaggtgacattccccgagcttgccagaaaagcttgcgtccagtcccaccatcgcccaagattgatcgaggttgggtatgtgtttttcagcttcaagatggtaaaacacttggactcaaaatttgagccaatctcccttccctccgaaagaggcgaataatagcagaggcttcaactgctgaactatagggtacgttacattaatgatacacttgtgagtatcagccctggataatataagtcaattaaacgaccaagataaaattgttcatatctcgctagcagcttaaaatataaatgtaataggagctatatctctgacagtattataatcaattgttattaagtaacccaaaccaaaagtgatgaagattaagaaaaacctacctcggctgagagagtgttttttcattaaccttcatcttgtaaacgttgagcaaaattgttaaaaatatgaggcgggttatattgcctactgctcctcctgaatatatggaggccatataccctgtcaggtcaaattcaacaattgctagaggtggcaacagcaatacaggcttcctgacaccggagtcagtcaatggggacactccatcgaatccactcaggccaattgccgatgacaccatcgaccatgccagccacacaccaggcagtgtgtcatcagcattcatccttgaagctatggtgaatgtcatatcgggccccaaagtgctaatgaagcaaattccaatttggcttcctctaggtgtcgctgatcaaaagacctacagctttgactcaactacggccgccatcatgcttgcttcatacactatcacccatttcggcaaggcaaccaatccacttgtcagagtcaatcggctgggtcctggaatcccggatcatcccctcaggctcctgcgaattggaaaccaggctttcctccaggagttcgttcttccgccagtccaactaccccagtatttcacctttgatttgacagcactcaaactgatcacccaaccactgcctgctgcaacatggaccgatgacactccaacaggatcaaatggagcgttgcgtccaggaatttcatttcatccaaaacttcgccccattcttttacccaacaaaagtgggaagaaggggaacagtgccgatctaacatctccggagaaaatccaagcaataatgacttcactccaggactttaagatcgttccaattgatccaaccaaaaatatcatgggaatcgaagtgccagaaactctggtccacaagctgaccggtaagaaggtgacttctaaaaatggacaaccaatcatccctgttcttttgccaaagtacattgggttggacccggtggctccaggagacctcaccatggtaatcacacaggattgtgacacgtgtcattctcctgcaagtcttccagctgtgattgagaagtaattgcaataattgactcagatccagttttatagaatcttctcagggatagtgataacatctatttagtaatccgtccattagaggagacacttttaattgatcaatatactaaaggtgctttacaccattgtcttttttctctcctaaatgtagaacttaacaaaagactcataatatacttgtttttaaaggattgattgatgaaagatcataactaataacattacaaataatcctactataatcaatacggtgattcaaatgttaatctttctcattgcacatactttttgcccttatcctcaaattgcctgcatgcttacatctgaggatagccagtgtgacttggattggaaatgtggagaaaaaatcgggacccatttctaggttgttcacaatccaagtacagacattgcccttctaattaagaaaaaatcggcgatgaagattaagccgacagtgagcgtaatcttcatctctcttagattatttgttttccagagtaggggtcgtcaggtccttttcaatcgtgtaaccaaaataaactccactagaaggatattgtggggcaacaacacaatgggcgttacaggaatattgcagttacctcgtgatcgattcaagaggacatcattctttctttgggtaattatccttttccaaagaacattttccatcccacttggagtcatccacaatagcacattacaggttagtgatgtcgacaaactagtttgtcgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccataaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaggctaccggttttggaaccaatgagacagagtacttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaacctcactagaaaaattcgcagtgaagagttgtctttcacagttgtatcaaacggagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctaacaacccttgccacaatctccacgagtccccaatccctcacaaccaaaccaggtccggacaacagcacccataatacacccgtgtataaacttgacatctctgaggcaactcaagttgaacaacatcaccgcagaacagacaacgacagcacagcctccgacactccctctgccacgaccgcagccggacccccaaaagcagagaacaccaacacgagcaagagcactgacttcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggagaagaactcgaagagaagcaattgtcaatgctcaacccaaatgcaaccctaatttacattactggactactcaggatgaaggtgctgcaatcggactggcctggataccatatttcgggccagcagccgagggaatttacatagaggggctaatgcacaatcaagatggtttaatctgtgggttgagacagctggccaacgagacgactcaagctcttcaactgttcctgagagccacaactgagctacgcaccttttcaatcctcaaccgtaaggcaattgatttcttgctgcagcgatggggcggcacatgccacattctgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttataattgcagttatcgctttattctgtatatgcaaatttgtcttttagtttttcttcagattgcttcatggaaaagctcagcctcaaatcaatgaaaccaggatttaattatatggattacttgaatctaagattacttgacaaatgataatataatacactggagctttaaacatagccaatgtgattctaactcctttaaactcacagttaatcataaacaaggtttgacatcaatctagttatctctttgagaatgataaacttgatgaagattaagaaaaaggtaatctttcgattatctttaatcttcatccttgattctacaatcatgacagttgtctttagtgacaagggaaagaagcctttttattaagttgtaataatcagatctgcgaaccggtagagtttagttgcaacctaacacacataaagcattggtcaaaaagtcaatagaaatttaaacagtgagtggagacaacttttaaatggaagcttcatatgagagaggacgcccacgagctgccagacagcattcaagggatggacacgaccaccatgttcgagcacgatcatcatccagagagaattatcgaggtgagtaccgtcaatcaaggagcgcctcacaagtgcgcgttcctactgtatttcataagaagagagttgaaccattaacagttcctccagcacctaaagacatatgtccgaccttgaaaaaaggatttttgtgtgacagtagtttttgcaaaaaagatcaccagttggagagtttaactgatagggaattactcctactaatcgcccgtaagacttgtggatcagtagaacaacaattaaatataactgcacccaaggactcgcgcttagcaaatccaacggctgatgatttccagcaagaggaaggtccaaaaattaccttgttgacactgatcaagacggcagaacactgggcgagacaagacatcagaaccatagaggattcaaaattaagagcattgttgactctatgtgctgtgatgacgaggaaattctcaaaatcccagctgagtcttttatgtgagacacacctaaggcgcgaggggcttgggcaagatcaggcagaacccgttctcgaagtatatcaacgattacacagtgataaaggaggcagttttgaagctgcactatggcaacaatgggaccgacaatccctaattatgtttatcactgcattcttgaatattgctctccagttaccgtgtgaaagttctgctgtcgttgtttcagggttaagaacattggttcctcaatcagataatgaggaagcttcaaccaacccggggacatgctcatggtctgatgagggtaccccttaataaggctgactaaaacactatataaccttctacttgatcacaatactccgtatacctatcatcatatatttaatcaagacgatatcctttaaaacttattcagtactataatcactctcgtttcaaattaataagatgtgcatgattgccctaatatatgaagaggtatgatacaaccctaacagtgatcaaagaaaatcataatctcgtatcgctcgtaatataacctgccaagcatacctcttgcacaaagtgattcttgtacacaaataatgttttactctacaggaggtagcaacgatccatcccatcaaaaaataagtatttcatgacttactaatgatctcttaaaatattaagaaaaactgacggaacataaattctttatgcttcaagctgtggaggaggtgtttggtattggctattgttatattacaatcaataacaagcttgtaaaaatattgttcttgtttcaagaggtagattgtgaccggaaatgctaaactaatgatgaagattaatgcggaggtctgataagaataaaccttattattcagattaggccccaagaggcattcttcatctccttttagcaaagtactatttcagggtagtccaattagtggcacgtcttttagctgtatatcagtcgcccctgagatacgccacaaaagtgtctctaagctaaattggtctgtacacatcccatacattgtattaggggcaataatatctaattgaacttagccgtttaaaatttagtgcataaatctgggctaacaccaccaggtcaactccattggctgaaaagaagcttacctacaacgaacatcactttgagcgccctcacaattaaaaaataggaacgtcgttccaacaatcgagcgcaaggtttcaaggttgaactgagagtgtctagacaacaaaatattgatactccagacaccaagcaagacctgagaaaaaaccatggctaaagctacgggacgatacaatctaatatcgcccaaaaaggacctggagaaaggggttgtcttaagcgacctctgtaacttcttagttagccaaactattcaggggtggaaggtttattgggctggtattgagtttgatgtgactcacaaaggaatggccctattgcatagactgaaaactaatgactttgcccctgcatggtcaatgacaaggaatctctttcctcatttatttcaaaatccgaattccacaattgaatcaccgctgtgggcattgagagtcatccttgcagcagggatacaggaccagctgattgaccagtctttgattgaacccttagcaggagcccttggtctgatctctgattggctgctaacaaccaacactaaccatttcaacatgcgaacacaacgtgtcaaggaacaattgagcctaaaaatgctgtcgttgattcgatccaatattctcaagtttattaacaaattggatgctctacatgtcgtgaactacaacggattgttgagcagtattgaaattggaactcaaaatcatacaatcatcataactcgaactaacatgggttttctggtggagctccaagaacccgacaaatcggcaatgaaccgcatgaagcctgggccggcgaaattttccctccttcatgagtccacactgaaagcatttacacaaggatcctcgacacgaatgcaaagtttgattcttgaatttaatagctctcttgctatctaactaaggtagaatacttcatattgagctaactcatatatgctgactcaatagttatcttgacatctctgctttcataatcagatatataagcataataaataaatactcatatttcttgataatttgtttaaccacagataaatcctcactgtaagccagcttccaagttgacacccttacaaaaaccaggactcagaatccctcaaacaagagattccaagacaacatcatagaattgctttattatatgaataagcattttatcaccagaaatcctatatactaaatggttaattgtaactgaacccgcaggtcacatgtgttaggtttcacagattctatatattactaactctatactcgtaattaacattagataagtagattaagaaaaaagcctgaggaagattaagaaaaactgcttattgggtctttccgtgttttagatgaagcagttgaaattcttcctcttgatattaaatggctacacaacatacccaatacccagacgctaggttatcatcaccaattgtattggaccaatgtgacctagtcactagagcttgcgggttatattcatcatactcccttaatccgcaactacgcaactgtaaactcccgaaacatatctaccgtttgaaatacgatgtaactgttaccaagttcttgagtgatgtaccagtggcgacattgcccatagatttcatagtcccagttcttctcaaggcactgtcaggcaatggattctgtcctgttgagccgcggtgccaacagttcttagatgaaatcattaagtacacaatgcaagatgctctcttcttgaaatattatctcaaaaatgtgggtgctcaagaagactgtgttgatgaacactttcaagagaaaatcttatcttcaattcagggcaatgaatttttacatcaaatgtttttctggtatgatctggctattttaactcgaaggggtagattaaatcgaggaaactctagatcaacatggtttgttcatgatgatttaatagacatcttaggctatggggactatgttttttggaagatcccaatttcaatgttaccactgaacacacaaggaatcccccatgctgctatggactggtatcaggcatcagtattcaaagaagcggttcaagggcatacacacattgtttctgtttctactgccgacgtcttgataatgtgcaaagatttaattacatgtcgattcaacacaactctaatctcaaaaatagcagagattgaggatccagtttgttctgattatcccaattttaagattgtgtctatgctttaccagagcggagattacttactctccatattagggtctgatgggtataaaattattaagttcctcgaaccattgtgcttggccaaaattcaattatgctcaaagtacactgagaggaagggccgattcttaacacaaatgcatttagctgtaaatcacaccctagaagaaattacagaaatgcgtgcactaaagccttcacaggctcaaaagatccgtgaattccatagaacattgataaggctggagatgacgccacaacaactttgtgagctattttccattcaaaaacactgggggcatcctgtgctacatagtgaaacagcaatccaaaaagttaaaaaacatgctacggtgctaaaagcattacgccctatagtgattttcgagacatactgtgtttttaaatatagtattgccaaacattattttgatagtcaaggatcttggtacagtgttacttcagataggaatctaacaccgggtcttaattcttatatcaaaagaaatcaattccctccgttgccaatgattaaagaactactatgggaattttaccaccttgaccaccctccacttttctcaaccaaaattattagtgacttaagtatttttataaaagacagagctaccgcagtagaaaggacatgctgggatgcagtattcgagcctaatgttctaggatataatccacctcacaaatttagtactaaacgtgtaccggaacaatttttagagcaagaaaacttttctattgagaatgttctttcctacgcacaaaaactcgagtatctactaccacaatatcggaacttttctttctcattgaaagagaaagagttgaatgtaggtagaaccttcggaaaattgccttatccgactcgcaatgttcaaacactttgtgaagctctgttagctgatggtcttgctaaagcatttcctagcaatatgatggtagttacggaacgtgagcaaaaagaaagcttattgcatcaagcatcatggcaccacacaagtgatgattttggtgaacatgccacagttagagggagtagctttgtaactgatttagagaaatacaatcttgcatttagatatgagtttacagcaccttttatagaatattgcaaccgttgctatggtgttaagaatgtttttaattggatgcattatacaatcccacagtgttatatgcatgtcagtgattattataatccaccacataacctcacactggagaatcgagacaacccccccgaagggcctagttcatacaggggtcatatgggagggattgaaggactgcaacaaaaactctggacaagtatttcatgtgctcaaatttctttagttgaaattaagactggttttaagttacgctcagctgtgatgggtgacaatcagtgcattactgttttatcagtcttccccttagagactgacgcagacgagcaggaacagagcgccgaagacaatgcagcgagggtggccgccagcctagcaaaagttacaagtgcctgtggaatctttttaaaacctgatgaaacatttgtacattcaggttttatctattttggaaaaaaacaatatttgaatggggtccaattgcctcagtcccttaaaacggctacaagaatggcaccattgtctgatgcaatttttgatgatcttcaagggaccctggctagtataggcactgcttttgagcgatccatctctgagacacgacatatctttccttgcaggataaccgcagctttccatacgtttttttcggtgagaatcttgcaatatcatcatctcgggttcaataaaggttttgaccttggacagttaacactcggcaaacctctggatttcggaacaatatcattggcactagcggtaccgcaggtgcttggagggttatccttcttgaatcctgagaaatgtttctaccggaatctaggagatccagttacctcaggcttattccagttaaaaacttatctccgaatgattgagatggatgatttattcttacctttaattgcgaagaaccctgggaactgcactgccattgactttgtgctaaatcctagcggattaaatgtccctgggtcgcaagacttaacttcatttctgcgccagattgtacgcaggaccatcaccctaagtgcgaaaaacaaacttattaataccttatttcatgcgtcagctgacttcgaagacgaaatggtttgtaaatggctattatcatcaactcctgttatgagtcgttttgcggccgatatcttttcacgcacgccgagcgggaagcgattgcaaattctaggatacctggaaggaacacgcacattattagcctctaagatcatcaacaataatacagagacaccggttttggacagactgaggaaaataacattgcaaaggtggagcctatggtttagttatcttgatcattgtgataatatcctggcggaggctttaacccaaataacttgcacagttgatttagcacagattctgagggaatattcatgggctcatattttagagggaagacctcttattggagccacactcccatgtatgattgagcaattcaaagtgttttggctgaaaccctacgaacaatgtccgcagtgttcaaatgcaaagcaaccaggtgggaaaccattcgtgtcagtggcagtcaagaaacatattgttagtgcatggccgaacgcatcccgaataagctggactatcggggatggaatcccatacattggatcaaggacagaagataagataggacaacctgctattaaaccaaaatgtccttccgcagccttaagagaggccattgaattggcgtcccgtttaacatgggtaactcaaggcagttcgaacagtgacttgctaataaaaccatttttggaagcacgagtaaatttaagtgttcaagaaatacttcaaatgaccccttcacattactcaggaaatattgttcacaggtacaacgatcaatacagtcctcattctttcatggccaatcgtatgagtaattcagcaacgcgattgattgtttctacaaacactttaggtgagttttcaggaggtggccagtctgcacgcgacagcaatattattttccagaatgttataaattatgcagttgcactgttcgatattaaatttagaaacactgaggctacagatatccaatataatcgtgctcaccttcatctaactaagtgttgcacccgggaagtaccagctcagtatttaacatacacatctacattggatttagatttaacaagataccgagaaaacgaattgatttatgacagtaatcctctaaaaggaggactcaattgcaatatctcattcgataatccatttttccaaggtaaacggctgaacattatagaagatgatcttattcgactgcctcacttatctggatgggagctagccaagaccatcatgcaatcaattatttcagatagcaacaattcatctacagacccaattagcagtggagaaacaagatcattcactacccatttcttaacttatcccaagataggacttctgtacagttttggggcctttgtaagttattatcttggcaatacaattcttcggactaagaaattaacacttgacaattttttatattacttaactactcaaattcataatctaccacatcgctcattgcgaatacttaagccaacattcaaacatgcaagcgttatgtcacggttaatgagtattgatcctcatttttctatttacataggcggtgctgcaggtgacagaggactctcagatgcggccaggttatttttgagaacgtccatttcatcttttcttacatttgtaaaagaatggataattaatcgcggaacaattgtccctttatggatagtatatccgctagagggtcaaaacccaacacctgtgaataattttctctatcagatcgtagaactgctggtgcatgattcatcaagacaacaggcttttaaaactaccataagtgatcatgtacatcctcacgacaatcttgtttacacatgtaagagtacagccagcaatttcttccatgcatcattggcgtactggaggagcagacacagaaacagcaaccgaaaatacttggcaagagactcttcaactggatcaagcacaaacaacagtgatggtcatattgagagaagtcaagaacaaaccaccagagatccacatgatggcactgaacggaatctagtcctacaaatgagccatgaaataaaaagaacgacaattccacaagaaaacacgcaccagggtccgtcgttccagtcctttctaagtgactctgcttgtggtacagcaaatccaaaactaaatttcgatcgatcgagacacaatgtgaaatttcaggatcataactcggcatccaagagggaaggtcatcaaataatctcacaccgtctagtcctacctttctttacattatctcaagggacacgccaattaacgtcatccaatgagtcacaaacccaagacgagatatcaaagtacttacggcaattgagatccgtcattgataccacagtttattgtagatttaccggtatagtctcgtccatgcattacaaacttgatgaggtcctttgggaaatagagagtttcaagtcggctgtgacgctagcagagggagaaggtgctggtgccttactattgattcagaaataccaagttaagaccttatttttcaacacgctagctactgagtccagtatagagtcagaaatagtatcaggaatgactactcctaggatgcttctacctgttatgtcaaaattccataatgaccaaattgagattattcttaacaactcagcaagccaaataacagacataacaaatcctacttggtttaaagaccaaagagcaaggctacctaagcaagtcgaggttataaccatggatgcagagacaacagagaatataaacagatcgaaattgtacgaagctgtatataaattgatcttacaccatattgatcctagcgtattgaaagcagtggtccttaaagtctttctaagtgatactgagggtatgttatggctaaatgataatttagccccgttttttgccactggttatttaattaagccaataacgtcaagtgctagatctagtgagtggtatctttgtctgacgaacttcttatcaactacacgtaagatgccacaccaaaaccatctcagttgtaaacaggtaatacttacggcattgcaactgcaaattcaacgaagcccatactggctaagtcatttaactcagtatgctgactgtgagttacatttaagttatatccgccttggttttccatcattagagaaagtactataccacaggtataacctcgtcgattcaaaaagaggtccactagtctctatcactcagcacttagcacatcttagagcagagattcgagaattaactaatgattataatcaacagcgacaaagtcggactcaaacatatcactttattcgtactgcaaaaggacgaatcacaaaactagtcaatgattatttaaaattctttcttattgtgcaagcattaaaacataatgggacatggcaagctgagtttaagaaattaccagagttgattagtgtgtgcaataggttctaccatattagagattgcaattgtgaagaacgtttcttagttcaaaccttatatttacatagaatgcaggattctgaagttaagcttatcgaaaggctgacagggcttctgagtttatttccggatggtctctacaggtttgattgaattaccgtgcatagtatcctgatacttgcaaaggttggttattaacatacagattataaaaaactcataaattgctctcatacatcatattgatctaatctcaataaacaactatttaaataacgaaaggagtccctatattatatactatatttagcctctctccctgcgtgataatcaaaaaattcacaatgcagcatgtgtgacatattactgccgcaatgaatttaacgcaacataataaactctgcactctttataattaagctttaacgaaaggtctgggctcatattgttattgatataataatgttgtatcaatatcctgtcagatggaatagtgttttggttgataacacaacttcttaaaacaaaattgatctttaagattaagttttttataattatcattactttaatttgtcgttttaaaaacggtgatagccttaatctttgtgtaaaataagagattaggtgtaataaccttaacatttttgtctagtaagctactatttcatacagaatgataaaattaaaagaaaaggcaggactgtaaaatcagaaataccttctttacaatatagcagactagataataatcttcgtgttaatgataattaagacattgaccacgctcatcagaaggctcgccagaataaacgttgcaaaaaggattcctggaaaaatggtcgcacacaaaaatttaaaaataaatctatttcttcttttttgtgtgtcca

SEQ ID NO: 12 - DNA, 10735 bp, Dengue virus; identical to SEQ ID NO: 1

SEQ ID NO: 13 - DNA, 10962 bp, West Nile virus
agtagttcgcctgtgtgagctgacaaacttagtagtgtttgtgaggattaacaacaattaacacagtgcgagctgtttcttggcacgaagatctcgatgtctaagaaaccaggagggcccggtaaaaaccgggctgtcaatatgctaaaacgcggtatgccccgcggattgtccttgataggactaaagagggctatgctgagtctgattgacgggaagggcccaatacgtttcgtgttggctcttttggcgtttttcagattcactgcaatcgctccgactcgtgcggtgctggacagatggagaggcgtcaacaaacaaacagcaatgaagcatctcttgagtttcaagaaagaactaggaactctgaccagtgccatcaaccgccggagcacaaaacaaaagaaaagaggaggcacagcgggctttactatcttgcttgggctgatcgcctgtgctggagctgtgaccctctcgaacttccagggcaaagtgatgatgacagtcaatgcaaccgatgtcactgacgtgattaccattccaacagctgctgggaaaaacctgtgcatcgtaagggctatggacgtaggatacctttgtgaggatactatcacttatgaatgtccggtcctagctgctggaaatgaccctgaagacattgactgctggtgcacgaaatcatctgtttacgtgcgctatggaagatgcacaaaaactcggcattcccgtcgaagcagaaggtctctgacagtccagacacatggagaaagtacactggccaacaagaaaggagcttggttggacagcacaaaagccacgagatatctggtgaagacagaatcatggatactgagaaacccgggctacgccctcgttgcagctgtcattggatggatgctaggaagcaacacaatgcaacgcgtcgtgtttgccattctattgctcctggtggcaccagcatacagcttcaactgtttaggaatgagtaacagagacttcctggagggagtgtctggagctacatgggttgatctggtactggaaggcgatagttgtgtgaccataatgtcaaaagacaagccaaccattgatgtcaaaatgatgaacatggaagcagccaacctcgcagatgtgcgcagttactgttacctagcttcggtcagtgacttgtcaacaagagctgcgtgtccaaccatgggtgaagcccacaacgagaaaagagctgaccccgccttcgtttgcaagcaaggcgttgtggacagaggatggggaaatggctgcggactgtttggaaaggggagcattgacacatgtgcgaagtttgcctgtacaaccaaagcaactggatggatcatccagaaggaaaacatcaagtatgaggttgccatatttgtgcatggcccgacgaccgttgaatctcatggcaagataggggccacccaggctggaagattcagtataactccatcggcgccatcttacacgctaaagttgggtgagtatggtgaggttacggttgattgtgagccacggtcaggaatagacaccagcgcctattacgttatgtcagttggtgagaagtccttcctggttcaccgagaatggtttatggatctgaacctgccatggagcagtgctggaagcaccacgtggaggaaccgggaaacactgatggagtttgaagaacctcatgccaccaaacaatctgttgtggctctagggtcgcaggaaggtgcgttgcaccaagctctggccggagcgattcctgttgagttctcaagcaacactgtgaagttgacatcaggacatctgaagtgtcgggtgaagatggagaagttgcagctgaagggaacaacatatggagtatgttcaaaagcgttcaaattcgctaggactcccgctgacactggccacggaacggtggtgttggaactgcaatataccggaacagacggtccctgcaaagtgcccatttcttccgtagcttccctgaatgacctcacacctgttggaagactggtgaccgtgaatccatttgtgtctgtggccacagccaactcgaaggttttgattgaactcgaacccccgtttggtgactcttacatcgtggtgggaagaggagaacagcagataaaccatcactggcacaaatctgggagcagcattggaaaggcctttaccaccacactcagaggagctcaacgactcgcagctcttggagatactgcttgggattttggatcagttggaggggttttcacctcagtggggaaagccatacaccaagtctttggaggagcttttagatcactctttggagggatgtcctggatcacacagggacttctgggagctcttctgttgtggatgggaatcaatgcccgtgacaggtcaattgctatgacgtttcttgcggttggaggagttttgctcttcctttcggtcaacgtccatgctgacacaggctgtgccattgatattggcaggcaagagctccggtgcggaagtggagtgtttatccacaacgatgtggaagcctggatggatcgttacaagttctacccggagacgccacagggcctagcaaaaattatccagaaagcacatgcagaaggagtctgcggcttgcgttccgtttccagactcgagcaccaaatgtgggaagccattaaggatgagctgaacaccctgttgaaagagaatggagtcgacttgagtgtcgtggtggaaaaacagaatgggatgtacaaagcagcaccaaaacgtttggctgccaccaccgaaaaactggagatgggttggaaggcttggggcaagagtatcatctttgcgccagaactagctaacaacacctttgtcatcgacggtcctgagactgaggaatgcccaacggccaaccgagcatggaacagtatggaggtagaggactttggatttggactgacaagcactcgcatgttcctgaggattcgggaaacgaacacaacggaatgcgactcgaagatcataggaaccgccgtcaagaacaacatggctgtgcatagtgatctatcatactggatagagagcggactcaacgacacctggaagcttgagagggcggttctaggagaagtcaaatcatgcacctggccagaaacccacactctgtggggtgatggagttctggaaagtgatctcatcatacccatcaccttggcaggacccagaagcaaccacaacaggagaccagggtacaaaactcagaaccaaggcccatgggatgaggggcgcgtcgagattgactttgactattgcccaggaacaacagtaactataagtgacagttgcgaacaccgtggacctgcggcacgcacaaccactgagagtgggaagctcatcacagactggtgctgcagaagttgcaccctccctccactgcgcttccagactgagaatggctgttggtatggaatggaaattcgacctacgcggcacgacgaaaagaccctcgtgcaatcgagagtgaatgcatacaacgccgacatgattgatccttttcagttgggccttatggtcgtgttcttggccacccaggaggtccttcgcaagaggtggacggccaagatcagcattccagctatcatgcttgcactcctagtcctagtgtttgggggtattacgtacactgatgtcctgcgatatgtcattctcgtcggcgccgcgtttgctgaagcaaactcaggaggagacgtcgtgcacttggcacttatggctacattcaagattcaaccagtctttctggtggcttcctttttgaaggcaaggtggaccaaccaagagagtattttgctcatgcttgcagctgctttcttccaaatggcttactatgacgccaagaatgttctgtcatgggaagtgcctgacgttttgaactctctctccgttgcgtggatgattctcagagctataagcttcaccaacacttcaaatgtggtggtgccgctgctggcccttttgacacctggattgaaatgcttaaaccttgatgtgtacagaattttgctactcatggttggagttggaagcctcatcaaagaaaaaaggagctctgcagcaaaaaagaaaggagcttgcctcatctgcctagcgctggcgtctacaggagtgttcaatccaatgatacttgcagctgggctaatggcttgcgaccccaaccgcaagcggggctggcctgctacagaagtgatgactgcagttggactcatgtttgccatcgttgggggtctggcagaacttgacatagattctatggctatccccatgaccatcgccggacttatgttcgcggcatttgtcatctctggaaagtcaacagacatgtggattgagaggacggctgacattacttgggagagtgatgctgaaatcacaggctctagcgaaagagtagatgtgaggctggatgatgatggaaattttcaactgatgaatgaccccggggcaccatggaaaatttggatgcttaggatggcctgcctggcgataagtgcctacacaccttgggcaattctcccctcggtcatcggattctggataacccttcagtacacaaagagaggaggtgttctttgggacacaccatcacccaaggagtacaagaagggtgataccaccactggcgtttacagaatcatgactcgaggtctgcttggcagttaccaagctggagccggagtgatggtagagggggtgttccacacactatggcacaccactaagggagctgctctcatgagtggtgagggacgtctggatccctactgggggagcgtgaaagaggaccgactttgctatggggggccatggaaactccaacataaatggaatggacatgatgaggtccaaatgattgtcgtggagccagggaaaaatgtgaaaaacgtccagaccaagcccggagtgtttaagacaccagaaggagaaattggggcagttacgctagactatcctaccggaacgtcaggttcccccattgtagacaaaaatggagatgtgattggattgtatgggaacggcgtcatcatgcctaatggttcatacataagcgccattgtgcaaggagagagaatggaagaaccggcaccagctggcttcgaacctgaaatgttgaggaagaaacagatcactgtccttgatctgcaccccggagcaggaaagacacgcaagatacttccccaaatcatcaaggaggccatcaacaaaagattgaggacggctgtgctggcacccaccagggtcgttgctgctgagatgtctgaggccctgagaggacttcccattcggtaccaaacctcagcagtgcacagagagcacagtggaaatgagatcgttgatgtcatgtgccatgccaccctcacacacaggctgatgtctccacacagagtccccaactacaacctgttcataatggatgaagcccatttcacggatccagcgagcatcgcagccagaggatacatagcaaccaaggttgaattgggcgaagccgccgcgattttcatgacggcaacgccacccgggacttctgacccctttccagagtctaatgctcctatctcggacatgcaaacagagatcccagacagagcctggaacactggatatgaatggataactgagtatgttggaaagaccgtttggtttgttccaagtgtgaaaatgggaaatgagattgccctctgtctgcaacgggcggggaagaaggttatccagctgaacagaaagtcctatgagacagagtaccccaagtgtaagaacgatgattgggattttgtcatcaccacagacatatcagaaatgggagccaacttcaaggcgagcagagtgatcgacagccgcaaaagcgtgaaacccaccatcattgaggaaggtgatggaagagtcatcctgggggaaccctcagccatcacggctgccagcgctgctcagcggagaggacgcataggaagaaacccatcacaagttggtgatgagtattgctatggagggcacacaaatgaggatgattccaactttgctcactggacagaggctcgcatcatgctagacaacatcaacatgccgaatggtctggtggctcaactatatcagcctgagcgcgagaaggtgtacaccatggacggggaatacaggctcagaggggaagaacggaagaacttccttgaattcctgagaacagctgatttaccagtctggctcgcttacaaagtggcagcagcaggaatatcataccatgaccggaaatggtgctttgatggacctcgaaccaacacgattcttgaagacaacaatgaagttgaagtcatcacgaagttgggtgagagaaagatcctaagacccaggtgggcagatgctagagtgtactcagaccatcaagctctaaagtccttcaaagattttgcatcggggaaacgatcacaaatcgggctcgttgaggtgctcgggagaatgcctgaacacttcatggtgaaaacttgggaggcattggacacgatgtatgtggtggcgaccgctgaaaaaggaggccgagctcacaggatggctcttgaggagctaccggacgcccttcagacaatagttttgattgcactattgagtgtgatgtccttaggtgtgttttttctactcatgcaaaggaagggcattggtaagattggcttgggaggagtaatcttaggagctgccacattcttctgctggatggctgaagtcccaggaacgaaaatagcaggcatgctcctgctttccctgctgctcatgattgttttgattccggagccggaaaagcagcgctcacagactgataaccagctcgccgtgttcttgatctgtgtgctcacactggtcggcgccgtggctgccaatgaaatgggctggctggacaagaccaagaatgacattggcagcctgttggggcacaggccagaagctagagagacgaccctgggagttgagagcttcttacttgatctgcggccggccacggcatggtcgctctatgccgtaacgacagccgttctcacccctttgctgaagcatctaatcacgtcagactacatcaacacttcgttgacctcaataaacgtccaagccagcgcgttgttcactttggccagaggcttcccttttgtggacgttggtgtgtcagctctcttgctggcggtcgggtgctggggtcaggtgactctgactgtgactgtgactgcagctgctctgctcttttgccactatgcttacatggtgccaggctggcaagcggaagccatgcgatctgcccagcggcggacagctgctggcatcatgaaaaatgtagtggtggatgggatcgtggccactgatgtacctgaacttgaacgaacaactccagtcatgcagaaaaaagttggacagatcatattgatcttggtatcaatggccgcggtggtcgtcaatccatcagtgagaaccgtcagagaggccggaattctgactacagcagcagcagtcaccctatgggagaatggtgctagttcagtgtggaatgcaacgacagctattggcctttgtcacatcatgcgaggaggatggctctcgtgtctctccatcatgtggactctcatcaaaaacatggagaaaccaggcctcaagaggggtggagccaaaggacgcacgctaggggaagtttggaaggagagactcaaccacatgacgaaggaagaatttaccagatacagaaaagaagccatcactgaagttgaccgctccgcagcaaaacatgctaggagagagggaaacatcactggaggccacccagtctcacggggaaccgcgaaattacggtggttagtggaaaggcgtttcctcgagccagtgggaaaggttgtggatctcgggtgtggtagaggcggctggtgctattacatggctacccagaagagggtacaggaagtgaaagggtacacgaaaggaggacctggccatgaagaaccacaactggtgcagagctatggttggaatattgttaccatgaagagtggagtcgacgtcttctacagaccatcagaagcgagcgacacactgctctgtgacattggagagtcatcgtcaagtgccgaggtagaagaacaccgcaccgtccgtgtcctggagatggtggaagattggttgcacagaggaccgaaggaattctgcatcaaagtgctatgcccttacatgcccaaagtgattgagaagatggaaacactccaaaggcgatatggaggtggccttataagaaaccccctttcacgcaactctacccatgagatgtactgggtgagccacgcttcaggcaatatcgtccactccgtcaacatgacaagccaggtgcttctggggaggatggaaaagaaaacatggaagggaccccagtttgaggaagatgtcaacttgggaagtggaacgcgggcagtagggaagcctctcctcaattctgatactagcaagatcaagaaccgaattgagaggctgaagaaagaatacagctccacatggcaccaggatgcgaaccacccctacaggacctggaactaccacggaagctatgaagtgaaaccaaccggctcagccagctcccttgtgaatggggtagtcagattactctcaaaaccatgggacactatcaccaatgtgaccacgatggccatgacagacaccactcctttcggtcaacaacgagtgttcaaggaaaaggtggacacaaaggctccagagcctccagaaggagtcaaatacgtcctcaatgagaccacgaactggctgtgggcttttttagcccgcgataagaaacccaggatgtgttcccgggaggaatttattggaaaagtcaacagtaatgccgccctaggagcgatgtttgaagaacagaaccaatggaagaacgcccgggaagctgtagaggatccaaagttttgggagatggtggatgaggagcgtgaagcgcatctccgtggagaatgcaacacctgcatctacaacatgatgggaaagagagagaagaagcctggagagttcggcaaagctaaaggcagcagagccatctggttcatgtggctgggggcccgcttcctggagtttgaagctctcggattcctcaatgaagaccactggctgggtaggaagaactcaggaggaggagttgaaggcttaggactgcagaagctcgggtacatcttgaaggaagttggaacaaagcctggaggaaaggtttacgctgatgataccgcaggctgggacacacgcatcaccaaagctgacctcgagaatgaagcgaaggttcttgaactgctggatggagaacatcgacgtttagcgcggtccatcatcgagctcacataccgacacaaagtcgtgaaagtgatgaggccagcggccgacgggaaaactgtgatggacgtcatctctagagaggatcagagaggaagcggtcaggtagtgacttacgccctgaacaccttcaccaatctagcagttcagctggtcagaatgatggagggggagggggtcattggacccgatgatgttgaaaaactgggaaaaggaaaaggccctaaggtcagaacctggctgtttgagaatggcgaggagcgtctcagtcgcatggccgtcagcggtgatgactgcgtggtgaaacctttggacgaccgcttcgccacatcactacacttcctaaatgctatgtcaaaggtccgcaaagacatccaggaatggaaaccctcgacggggtggtatgactggcagcaggttccattctgttcaaaccatttcacggaactgatcatgaaggacggcaggacgctggtggtcccgtgtcgtggacaagacgagttgattggacgtgccaggatctctccaggggctggatggaatgtgcgcgacaccgcctgcctggcgaagtcatacgcgcagatgtggctgctgctttatttccaccgtagagacctgagattgatggccaatgccatctgttccgctgtgcctgccaactgggttcccacagggcgtaccacttggtcgatccacgcaaaaggagaatggatgacgacggaagacatgctcgcagtctggaacagagtgtggattgaggagaatgagtggatggaagacaaaacaccagttgagaggtggagtgatgttccatactctggaaagagagaggacatttggtgtggcagtttgatcggcacacgaacccgcgccacttgggctgaaaatatccatgtggcaatcaatcaggtccgttcagtgattggagaagagaagtatgtggattacatgagctccttgaggaggtatgaagacaccattgtagtggaggacactgttttgtaaaagatagtattatagttagtttagtgtaaataggatttattgagaatggaagtcaggccagattaatgctgccaccggaagttgagtagacggtgctgcctgcggctcaaccccaggaggactgggtgaccaaagctgcgaggtgatccacgtaagccctcagaaccgtctcggaaggaggaccccacgtgctttagcctcaaagcccagtgtcagaccacactttaatgtgccactctgcggagagtgcagtctgcgatagtgccccaggtggactgggttaacaaaggcaaaacatcgccccacgcggccataaccctggctatggtgttaaccagggagaagggactagaggttagaggagaccccgcgtaaaaaagtgcacggcccaacttggctgaagctgtaagccaagggaaggactagaggttagaggagaccccgtgccaaaaacaccaaaagaaacagcatattgacacctgggatagactaggggatcttctgctctgcacaaccagccacacggcacagtgcgccgacataggtggctggtggtgctagaacacaggatct

(19) United States
(12) Patent Application Publication
(10) Pub. No.: US 2023/0140799 A1
(43) Pub. Date: May 4, 2023

(54) DECOY TRANSCRIPTS FOR TREATMENT OF ssRNA VIRAL INFECTION

(71) Applicant: TEL HASHOMER MEDICAL RESEARCH INFRASTRUCTURE AND SERVICES LTD., Ramat Gan (IL)

(72) Inventors: Eitan GIAT, Talmon (IL); Yahel GIAT, Revava (IL); Itamar GOLDSTEIN, Tel Aviv (IL)

(21) Appl. No.: 17/910,293

(22) PCT Filed: Apr. 11, 2021
(86) PCT No.: PCT/IL2021/050413

§ 371 Date: Sep. 8, 2022

Related U.S. Application Data
(60) Provisional application No. 63/008,756, filed on Apr. 12, 2020.

(51) Int. Cl. A61K 31/7105; C12N 15/86; A61P 31/14

(52) U.S. Cl. CPC A61K 31/7105; C12N 15/86; A61P 31/14; C12N 2770/20022; C12N 2770/20032; C12N 2770/20043; C12N 2770/20062; C12N 2310/11

(57) ABSTRACT

There is provided a decoy transcript derived from a ssRNA virus (WV), the transcript comprising at least one of a 5′UTR of the WV, a genomic packaging signal (GPS) of the WV, a 3′UTR of the WV, and an exogenous stop codon.

### TECHNOLOGICAL FIELD

Provided herein are decoy transcripts and vectors comprising same for attenuating/treating viral infection, in particular for treatment of infections with single stranded (ss)RNA viruses.

### BACKGROUND

Viruses pose a threat to human health and at times may even lead to worldwide pandemics as in the case of the zoonotic Coronaviruses (CoV), e.g. SARS-CoV-2.

CoV are a large family of positive single-stranded (ss)RNA viruses causing respiratory illness in humans, ranging from the common cold to fatal pneumonia coupled with the cytokine release syndrome. CoV are zoonotic and transmit between animals and humans. In the last decade, two previous outbreaks of such zoonotic CoV with high fatality rates developed, the Middle East Respiratory Syndrome (MERS-CoV) and Severe Acute Respiratory Syndrome (SARS-CoV).

The COVID-19 disease is caused by a new strain, previously unidentified in human, designated SARS-CoV-2 that most likely originated from bats and was first isolated in Chinese patients in December 2019. In 2020, global economy and mass transportation exposes humanity to new zoonotic RNA viruses, such as SARS-Cov1/2 (ebolavirus and others) for which the vast majority of humans are not immunized, providing pandemic potential, as observed in the 2019 zoonotic CoV outbreak.

Current anti-viral therapy focuses on vaccination and targeting specific viral proteins. The main flaw of this approach is the quick evolution of a virus, which allows it to evade vaccination and develop resistance to anti-viral drugs. In the case of a resistant strain or a new epidemic, it takes precious time to develop a new traditional vaccine or treatment. Moreover, since viral infections typically involve a relatively long incubation period and a large portion of infected individuals are asymptomatic, it is often difficult to effectively treat all subject's in need thereof, whether as a treatment or as a mean to prevent new infections.

Thus, novel unorthodox antiviral therapeutics are urgently needed for the treatment of these newly introduced zoonotic viruses against which vaccines and effective antiviral therapies have not been available.

### SUMMARY OF THE INVENTION

There is provided herein a parasitic pseudo-viral transcript (PSCT, also referred to herein as a “decoy transcript”, or as a “PSCT particle”) that is harmless and unable to replicate in the absence of the wild type virus from which it is a decoy of, and it includes all necessary sequences for efficient replication and packaging.

The purpose of the PSCT is to act as a parasite that competes with the wild-type virus (WV) for its replication and encapsulation machinery. As a result, within a few virus replication cycles, the majority of virions will encapsulate mostly the decoy transcript, thus slowing down the spread of the WV potentially even to the point of its eradication by the host immune system.

The PSCT may advantageously be utilized in the treatment of infections caused by ssRNA viruses that employ RdRp for replication, such as for example coronaviruses. Further, the PSCT may also provide a solution for other viruses particularly in the setting of a global outbreak (e.g. pandemic).

Optionally, the PSCT may include short anti-sense sequences targeting the WV genomic mRNA (gRNA), as further elaborated herein below.

In summary, this disclosure provides a feasible, efficient and safe treatment that can potentially treat large populations with minimal intervention, utilizing a parasitic short synthetic mRNA vector that hijacks the WV machinery to spread within the host, as well as to other individuals infected with the WV.

According to some embodiments, the hereindisclosed treatment may overcome viral resistance (e.g. by spontaneous evolution of the PSCT that may evolve faster than the virus or by redesigning of the PSCT transcript based on newly discovered mutations), allowing a quick response to new divergent strains causing pandemics.

The PSCT has at least one of the following advantageous characteristics (see also FIG. 1):
1. The PSCT is a short synthetic RNA transcript, homologues to pre-selected small parts of the gRNA of a relevant ssRNA WV. According to some embodiments, the PSCT is an “obligate parasite”, .i.e. fully dependent on WV encoded proteins for its replication and packaging into nascent viral capsids and importantly lacks the capacity to replicate and/or produce viral particles in cells that are not co-infected by the WV virus. In an embodiment, within uninfected cells the PSCT will be eliminated by ubiquitous cellular pathways of RNA degradation.
2. The PSCT contains WV replication recognition sequences. In an embodiment, WV replication recognition sequences may mediate optimal recognition by the WV-encoded proteins, such as for example RdRp, the nucleocapsid (N) protein and the membrane (M) protein. In an embodiment, PSCT competes with the WV for at least one of the functions of viral replication and viral packaging.
3. According to some embodiments the PSCT may replicate at a higher rate than the WT gRNA. In an embodiment, PSCT comprises a short sequence with optimal affinity for WV RdRp and N-protein (see FIG. 1).
4. Optionally, the PSCT may include sequences that encode for small subgenomic (sg) sequences. In an embodiment, these sg transcripts include anti-sense 10-35 nt long sequences, which are complementary to the WV gRNA. In an embodiment, sg anti-sense sg sequences may hybridize to conserved sequences within the WV gRNA. Without being bound by any theory, the anti-sense transcripts may interfere with WV genome replication and/or induce degradation of WV gRNA. In an embodiment, sg antisense transcripts have minimal impact on PSCT functions, such as for example PSCT packaging into viral capsids. In an embodiment, degradation of WV gRNA induced by anti-sense transcripts may be mediated by host cellular RNAses.

Further, the PSCT may be characterized by at least one of the structural elements detailed below.
a) The 5′ end of the transcript is derived from the WV. The 5′ end includes at least one of: the WV leader sequence; and at least a part or parts of the WV untranslated region (UTR) that contain stem loop structures and the genomic packaging signal (GPS) of the WV.
   Stem loop structures are required for RNA replication by the RdRp, while GPS is required, for effective recognition by N protein and subsequent encapsulation.
   The 5′ end of the PSCT is modified in relation to the WV to include one or more of the following:
      i. A stop codon positioned about 6-500, 50-500, 100-300 or 150-250 (e.g. 207) nucleotides downstream from the first start codon. Each possibility is separate embodiment. The first stop codon or any stop codon will be at a position which will preserve the secondary structure necessary for recognition by the viral proteins. The stop codon ensures that the PSCT itself does not produce any WV proteins. The stop codon may be positioned in the closest possible proximity to the start codon in order for the ribosome to rapidly disassemble from the PSCT so that cellular resources are not wasted on synthesizing irrelevant long peptides. According to some embodiments, the stop codon allows disassembly of the ribosome without changing RNA secondary structure.
      ii. The 5′ end optionally includes one or more additional stop codons in frame shift to the first stop codon. Stop codons in frameshift may be a safeguard mechanism in case of a frame shift mutation downstream to the first stop codon.
      iii. The 5′ end optionally further includes a GPS sequences for recognition by the N-protein.
      iv. Optionally the transcript may further include one or more short anti-sense sequences. In an embodiment, each short anti-sense sequence may be flanked by a leader and a transcription regulatory sequence (TRS) required to guide the RdRp to synthesize sg sequences, also referred to as short strands. According to some embodiments, the short strands will not contain a start codon and will not translate into proteins. Alternatively at least some of the sg strands may be translated into peptides or proteins capable of inhibiting the virus and/or promoting PSCT spread. The short strands may inhibit replication of the WV, for example by pairing with the WV gRNA. and forming dsRNA secondary structures, which cannot be transcribed by viral RdRp that recognizes only ssRNA. Optionally, the antisense sequences may further serve as a substrate for cellular dsRNases.
b) The 3′ end of the PSCT transcript is derived from the WV. In an embodiment, the 3′end of the PSCT includes at least part of the WV 3′UTR. In an embodiment, the 3′end of the PSCT is a polyA tail. The 3′UTR may further contain RNA sequences predicted to be required for optimal replication of PSCT.
c) The PSCT generally does not encode WV proteins. WV proteins not encoded by the PSCT include, but are not limited to RdPd and various structural proteins such as N-protein, spike (S) protein and others. Further the PSCT generally does not encode non-structural proteins, such as accessory proteins involved in viral virulence in the host.

The PSCT cycle is briefly described below.

The PSCT enters the host cell (the first entry will be discussed herein below).

If the host cell is not infected, the PSCT transcript is degraded by the host cell.

If the host cell is infected by a WV (e.g. SARS-CoV-2), the RdRp of the WV should recognize the PSCT and bring about the PSCT replication. Optionally, in PSCT variants bearing WV antisense sequences, the antisense sequences may be transcribed. These custom-designed sg strands may function as partial inhibitors of WV gRNA replication and/or induce its degradation.

The replicated PSCT strands may be recognized and bind the N-protein of the WV, after which N-protein will bind to the M-protein and other relevant viral proteins. Binding of the PSCT strands to the N-, M- and other WV proteins result in PSCT-containing virions to be assembled. Due to the rapid replication of the PSCT, the PSCT reduces the amount of RdRp-, N- and M-proteins available to the WV transcript and thereby slows gRNA WV replication and encapsulation. WV inhibition continues in additional host cells through secretion of PSCT-containing virions from the host cell, and infection of additional cells. Infection of additional cells may reduce total WV gRNA load in the host.

As a further advantage, PSCT is transmissible, for example it may be transmitted from one subject to another by infection. That is, if a family or a large population is infected, by treating one family member or a few individuals in the population with the PSCT, the entire family/population (or at least large parts thereof) may be infected by the PSCT-containing non-pathogenic virions that may be transmitted concurrently with the WV, or independent of the WV, within members of the family/population. Thus, in an embodiment, the PSCT may be used as a treatment for a large group of subjects, such as for example a population. In an embodiment, the PSCT may be effective when treating third world countries and/or rapidly emerging epidemics/pandemics, especially when the viral infection is characterized by having a long incubation period and/or involving subjects with asymptomatic infection, making it hard to identify infected subjects to limit further transmission.

In addition, due to high replication rate, RdPd-induced mutation rate may be higher in a fast-replicating PSCT as compared to a much longer (e.g. 20-fold longer) WV gRNA. Accordingly, in an embodiment the PSCT may be self-adapting and it may compete with mutant WV gRNAs. In a further embodiment, treatment with a PSCT may be self-sufficient and avoid the need for repeated treatment, or avoid the need for treatment with new versions of a PSCT.

According to some embodiments, there is provided a viral decoy transcript derived from a ssRNA virus (WV), the transcript comprising a 5′ end comprising a 5′UTR of the WV, a genomic packaging signal (GPS) of the WV, a 3′UTR of the WV, an extrinsic stop codon, and a poly-A tail. It is understood that the stop codon used may be any of the UAG, UAA, UGA stop codons. As such, any specific stop codon disclosed in any of the sequences set forth herein may be substituted with any of the other stop codons of the genetic code. According to some embodiments, the transcript may include a stop-codon repeat. According to some embodiments, the stop-codon repeat may include at least two stop codons which are not in the same reading frame.

According to some embodiments, the decoy transcript does not encode a WV RdRp and/or WV N-protein. According to some embodiments, the decoy transcript does not encode any WV protein.

According to some embodiments, the 5′ end of the decoy transcript is capped. According to some embodiments, the decoy transcript includes a nucleotide sequence having at least 80% sequence similarity to the nucleotide sequence set forth in SEQ ID NO: 2 or SEQ ID NO: 6.

According to some embodiments, the decoy transcript includes a nucleotide sequence having at least 80% sequence similarity to any one or more of the nucleotide sequences set forth in SEQ ID NO: 3-5 and 7.

According to some embodiments, the decoy transcript may be derived from any one of the SEQ ID NO: 1 and 9-13.

According to some embodiments, the decoy transcript further includes one or more additional stop codons, in frame shift to the first stop codon.

According to some embodiments, the decoy transcript further includes one or more additional GPS sequences.

According to some embodiments, the decoy transcript further includes one or more short WV specific antisense sequences. According to some embodiments, the antisense sequence may be flanked by at least one of a leader and a transcription regulatory sequence (TRS).

According to some embodiments, the genome of the WV from which the decoy transcript is derived includes at least 20,000 nucleotides. According to some embodiments, the WV from which the decoy transcript has a viral genome of 20-40 kilobases.

According to some embodiments, the ratio in the length of the decoy transcript relative to the WV is at least 1:15.

According to some embodiments, the WV from which the decoy transcript is derived is a zoonotic virus. According to some embodiments, the WV from which the decoy transcript is derived is a positive-sense single stranded RNA virus. According to some embodiments, the WV may be any one or more of Severe Acute Respiratory Syndrome Coronavirus (SARS-CoV), a Middle East respiratory syndrome-related coronavirus (MERS-CoV), Severe acute respiratory syndrome coronavirus 2 (SARS-CoV-2), Hepatitis C virus (HCV), West Nile virus, dengue virus, the common cold rhinoviruses, respiratory syncytial virus (RSV), parainfluenza viruses, influenza viruses, Ebola virus, Marburg virus. Each possibility is a separate embodiment. According to some embodiments, the WV from which the decoy transcript is derived is a coronavirus. According to some embodiments, the coronavirus may be any one of SARS-CoV, SARS-CoV-2 virus or MERS-CoV. According to some embodiments, the corona virus is a SARS-CoV-2 virus.

According to some embodiments, the decoy transcript is an isolated RNA molecule.

According to some embodiments, there is provided a vector comprising the herein disclosed decoy transcript.

According to some embodiments, the vector further includes a promoter transcriptionally associated with decoy transcript. According to some embodiments, the promoter is a constitutively active promoter, an inducible promoter and/or tissue specific. Each possibility is a separate embodiment.

According to some embodiments, there is provided a cell or cell population including the herein disclosed decoy transcript or the herein disclosed vector.

According to some embodiments, the cell or cell population is an epithelial cell or any other target cell of the virus. According to some embodiments, the cell or cell population is an angiotensin converting enzyme 2 (ACE2) (or dipeptidyl-peptidase 4 (DPP4) or aminopeptidase N (APN) or any receptor targeted by the virus) surface expressing cell/cell population.

According to some embodiments, there is provided a composition including the herein disclosed decoy transcript and a suitable transport vehicle and/or carrier.

According to some embodiments, the carrier is water.

According to some embodiments, the composition is suitable for administration through aerosol.

According to some embodiments, the transport vehicle is a transcription vector. According to some embodiments, the transcription vector is any one of an adeno-viral vector or a lentiviral vector.

According to some embodiments, the transport vehicle is an in-vitro generated virion.

According to some embodiments, the transport vehicle is a liposome or a lipid nanoparticle.

According to some embodiments, the composition is formulated for oral and/or nasal administration. According to some embodiments, the composition is formulated for administration via inhalation.

According to some embodiments, there is provided a method for treating, attenuating and/or inhibiting spread of a ssRNA viral infection in a subject, the method comprising providing to the subject the herein disclosed decoy transcript or the herein disclosed composition.

According to some embodiments, the treated subject is a subject infected with the WV.

According to some embodiments, there is provided a method for treating a cell or a cell population infected with a ssRNA virus, the method including providing to the cell the herein disclosed decoy transcript or the herein disclosed composition.

According to some embodiments, the cell or cell population is infected with the WV.

According to some embodiments, the providing to the cell the decoy transcript includes expressing the decoy transcript in the cell.

According to some embodiments, there is provided a kit for treating, attenuating and/or inhibiting spread of a ssRNA viral infection, the kit comprising a dosage form of herein disclosed the decoy transcript or of the herein disclosed composition and instructions for use of same in the treating, attenuating and/or inhibiting spread of a ssRNA viral infection.

According to some embodiments, the dosage form may be any one of nose drops, a nasal spray, a throat sprays a sprayable liquid or an inhalant. Each possibility is a separate embodiment.

According to some embodiments, the kit further includes a device for delivery of the dosage form. According to some embodiments, the device may be a nebulizer, an inhaler or an atomizer. Each possibility is a separate embodiment.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

The invention will now be described in relation to certain examples and embodiments with reference to the following illustrative figures so that it may be more fully understood.

FIG. 1 is a schematic representation of a SARS-like CoV genome (and other ssRNA virus genomes), and of two embodiments of the herein disclosed decoy transcript (PSCT), Decoy transcript (option 1) and Decoy transcript (option 2). The terms “ORF 1a and ORF 1b” refer to the section of the transcript encoding the proteins vital for gRNA replication and transcription (e.g, RdRp). Each of ORF 1a and ORF 1b encodes multiple transcripts. ORF 1b is typically frameshifted relative to ORF 1a. “S-protein” refers to the section of the transcript encoding the spike protein, “E-protein” refers to the section of the transcript encoding the envelope protein, “M protein” refers to the section of the transcript encoding the membrane protein, and “N-protein” refers to the section of the transcript encoding the nucleocapsid protein. In an embodiment, Decoy transcripts are devoid of SARS-CoV protein-encoding sequences, for example sequences encoding structural proteins, RdR, or accessory proteins. Decoy transcripts include one or more stop codons not found in the WV genome. Optionally a decoy transcript (option 2) may include WV-targeting complementary TRS flanked inhibitory antisense or peptide sequences.

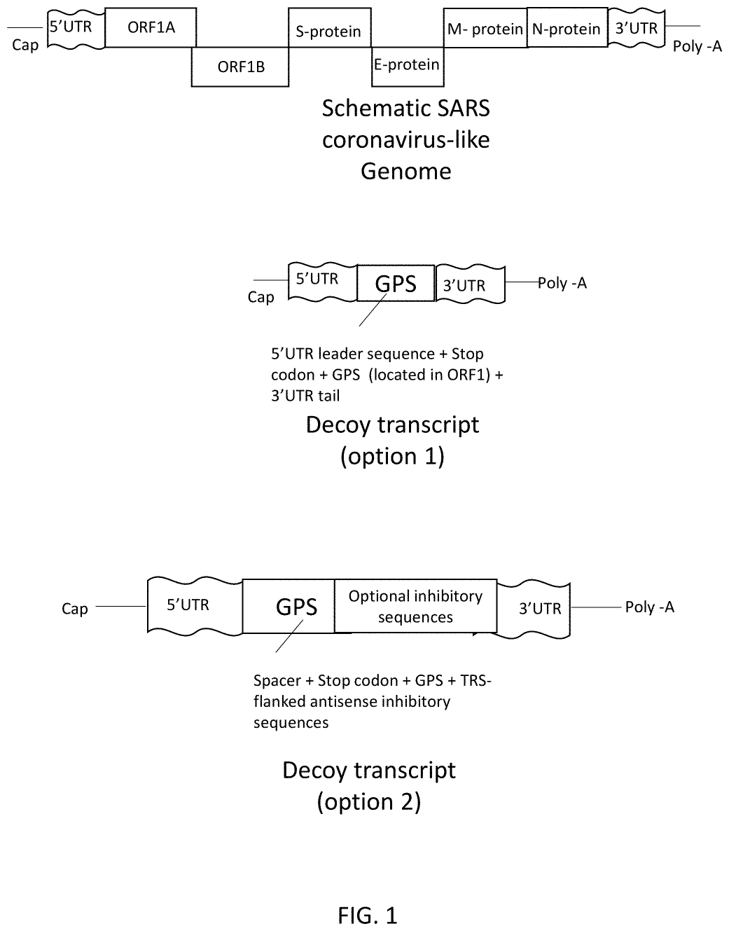

FIG. 2 is a diagram depicting the key characteristics of the therapeutic effects of the herein disclosed decoy transcript.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

### Definitions

The following definitions and methods are provided to better define the present invention and to guide those of ordinary skill in the art in the practice of the present invention. Unless otherwise noted, terms are to be understood according to conventional usage by those of ordinary skill in the relevant art.

Unless defined otherwise, all technical and scientific terms used herein have the same meaning as is commonly understood by one of skill in the art to which the invention(s) belong. All patents, patent applications, published applications and publications, GenBank sequences, databases, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

Where a term is provided in the singular, the inventors also contemplate aspects of the invention described by the plural of that term.

CoV-SARS-2, is a positive-sense ssRNA virus (see PMID 25720466, or PMID 31987001), having as genetic material the sequence denoted by SEQ ID NO: 1; NCBI Reference Sequence: NC_045512.2, genbank MN908947.3 or any variant or mutant strain thereof.

The viral cycle stages of CoV-SARS-2 are described in short below:
1. The viral ssRNA is inserted to the cytosol of a host cell.
2. The ssRNA is initially translated by the cellular ribosome to encode the long polyproteins, pp1a and pp1ab, which are further processed into a group of mature products including the important enzyme, the RNA dependent RNA polymerase (RdRp) and other non-structural proteins.
3. RdRp then synthesizes both genomic (replicase proteins) and sub-genomic RNA (sgRNA) transcripts (encoding structural and accessory proteins), resulting in the replication of the virus and expression of the vital viral structural genes, including the N-protein and other structural genes. sgRNA is synthesized through a mechanism that requires conserved TRS sequences.
4. The N protein binds the viral genome in a beads-on-a-string type conformation. A short cis-acting genome packaging signal (GPS) is both necessary and sufficient to facilitate recognition by the nucleocapsid (N) protein and mediate specific gRNA encapsulation.
5. Viral particles are then assembled and newly formed virions (including the envelope containing the Spike protein that allows the virions to infect other cells) bud to infect additional cells.

The terms “subject”, “patient” or “individual” generally refer to a human, although the methods of the invention are not necessarily limited to humans and should be useful in other mammals.

As referred to herein, the terms “polynucleotide molecules”, “oligonucleotide”, “polynucleotide”, “nucleic acid” and “nucleotide” sequences may interchangeably be used. The terms are directed to polymers of deoxyribonucleotides (DNA), ribonucleotides (RNA), and modified forms thereof in the form of a separate fragment or as a component of a larger construct, linear or branched, single stranded (ss), double stranded (ds), triple stranded (ts), or hybrids thereof. The term also encompasses RNA/DNA hybrids. The polynucleotides may be, for example, sense and antisense oligonucleotide or polynucleotide sequences of DNA or RNA. The DNA or RNA molecules may be, for example, but are not limited to: complementary DNA (cDNA), genomic DNA, synthesized DNA, recombinant DNA, or a hybrid thereof or an RNA molecule such as, for example, mRNA, shRNA, siRNA, miRNA, and the like. Accordingly, as used herein, the terms “polynucleotide molecules”, “oligonucleotide”, “polynucleotide”, “nucleic acid” and “nucleotide” sequences are meant to refer to both DNA and RNA molecules. The terms further include oligonucleotides composed of naturally occurring bases, sugars, and covalent inter nucleoside linkages, as well as oligonucleotides having non-naturally occurring portions, which function similarly to respective naturally occurring portions.

Unless otherwise stated, nucleotide sequences in the text of this specification are given, when read from left to right, in the 5′ to 3′ direction. The nomenclature used herein is that required by Title 37 of the United States Code of Federal Regulations § 1.822 and set forth in the tables in WIPO Standard ST.25 (1998), Appendix 2, Tables 1 and 3.

The terms “Upstream” and “Downstream”, as used herein refers to a relative position in a nucleotide sequence, such as, for example, a DNA sequence or an RNA sequence. As well known, a nucleotide sequence has a 5′ end and a 3′ end, so called for the carbons on the sugar (deoxyribose or ribose) ring of the nucleotide backbone. Hence, relative to the position on the nucleotide sequence, the term downstream relates to the region towards the 3′ end of the sequence. The term upstream relates to the region towards the 5′ end of the strand.

As used herein, the term “homolog” may refer to a polynucleotide having substantially from about 70% to about 99% sequence identity, or more preferably from about 80% to about 99% sequence identity, or most preferable from about 90% to about 99% sequence identity, to about 99% sequence identity, to the referent nucleotide sequences of a referent polynucleotide molecule. Each possibility is a separate embodiment.

As used herein, the term “sequence identity”, “sequence similarity” or “homology” is used to describe sequence relationships between two or more nucleotide sequences. The percentage of “sequence identity” between two sequences is determined by comparing two optimally aligned sequences. A sequence that is identical at every position in comparison to a reference sequence is said to be identical to the reference sequence and vice-versa. A first nucleotide sequence when observed in the 5′ to 3′ direction is said to be a “complement” of, or complementary to, a second or reference nucleotide sequence observed in the 3′ to 5′ direction if the first nucleotide sequence exhibits complete complementarity with the second or reference sequence. As used herein, nucleic acid sequence molecules are said to exhibit “complete complementarity” when every nucleotide of one of the sequences read 5′ to 3′ is complementary to every nucleotide of the other sequence when read 3′ to 5′. A nucleotide sequence that is complementary to a reference nucleotide sequence will exhibit a sequence identical to the reverse complement sequence of the reference nucleotide sequence. These terms and descriptions are well defined in the art and are easily understood by those of ordinary skill in the art.

As referred to herein, the term “complementarity” is directed to base pairing between strands of nucleic acids. As known in the art, each strand of a nucleic acid may be complementary to another strand in that the base pairs between the strands are non-covalently connected via two or three hydrogen bonds. Two nucleotides on opposite complementary nucleic acid strands that are connected by hydrogen bonds are called a base pair. According to the Watson-Crick DNA base pairing, adenine (A) forms a base pair with thymine (T) and guanine (G) with cytosine (C). In RNA, thymine is replaced by uracil (U). The degree of complementarity between two strands of nucleic acid may vary, according to the number (or percentage) of nucleotides that form base pairs between the strands. For example, “100% complementarity” indicates that all the nucleotides in each strand form base pairs with the complement strand. For example, “95% complementarity” indicates that 95% of the nucleotides in each strand from base pair with the complement strand. The term sufficient complementarity may include any percentage of complementarity from about 30% to about 100%.

As used herein, the term “frameshift” the addition or deletion of one or more nucleotides in a strand of DNA and/or RNA which shifts the codon triplets of the genetic code. Due to the triplet nature of gene expression by codons, the insertion or deletion can change the reading frame. Accordingly, with specific reference to stop codons which are frame shifted relative to each other refers to stop codons each recognized in a different possible reading frames.

As used herein, a “virus” refers to any of a large group of infectious entities that cannot grow or replicate without a host cell. Viruses typically contain a protein coat surrounding an RNA or DNA core of genetic material, but no semipermeable membrane, and are capable of growth and multiplication only in living cells.

As used herein, the terms “wild type virus” and “WV” refer to the virus from which the herein disclosed decoy transcript is derived. “Wild type virus” and “WV” further refer to an infectious virus that generates a disease in infected subjects, and which disease may be treated upon administration of therapeutic dosages of a PSCT as described herein.

According to some embodiments, the WV is a positive-sense single-stranded RNA virus. As used herein, the terms “positive-sense single-stranded RNA virus” and “(+)ssRNA virus” refers to a virus that uses positive sense single stranded RNA as its genetic material. Single stranded RNA viruses are classified as positive or negative depending on the sense or polarity of the RNA. The positive-sense viral RNA genome can serve as messenger RNA and can be translated into protein in the cytosol of the host cell without entering the nucleus of the host cell. Positive-sense RNA viruses account for a large fraction of known viruses, including many pathogens such as the hepatitis C virus, West Nile virus, dengue virus, SARS and MERS coronaviruses, and SARS-CoV-2 as well as less clinically serious pathogens, such as the rhinoviruses that cause the common cold. In an embodiment the WV virus is a virus from the Flaviviridae family of viruses. In an embodiment the WV virus is a virus from the Coronaviridae family of viruses.

According to some embodiments, the WV is a coronavirus. As used herein, the term “coronavirus” refers to a family of enveloped, positive-sense, single-stranded RNA viruses with a viral genome of 26-32 kilobases in length. According to some embodiments, the coronavirus may be a SARS, MERS and/or SARS-CoV-2 virus.

As used herein, the term “SARS-CoV-2” is directed to a pleomorphic RNA virus of the Corona genus Coronavirus in the Coronaviridae. When infecting humans, the SARS-CoV-2 virus may result in the COVID-19 condition. The SARS-CoV-2 genome is represented by the nucleic acid sequence as set forth by Accession No.: NC_045512.2, and herein as SEQ ID NO.1.

As used herein, “amplification of a virus in a host cell” means that the virus replicates in the cell to sustain the virus or increase the amount of virus in the cell. As used herein, a “host cell” or “target cell” are used interchangeably to mean a cell that can be infected by a virus. According to some embodiments, the host cell is a cell expressing ACE2 or a homolog thereof on its cell surface or a homologue thereof. According to some embodiments, the cell is an epithelial cell.

According to some embodiments, the WV may be a cytocidal virus. As used herein, the term “cytocidal virus” refers to a virus which when infecting a host cell, the virus kills the host cell through changes in cell morphology, in cell physiology, and/or the biosynthetic events. According to some embodiments, the changes to the host cell are necessary for efficient virus replication. According to some embodiments, the WV may be cytocidal to a large number of cells.

According to some embodiments, infection with the WV has a cytopathic effect. As used herein, the terms “cytopathic effect”, “cytopathogenic effect” and CPE may be used interchangeably and refer to structural changes in host cells that are caused by viral invasion. In some embodiments, the infecting virus causes lysis of the host cell or cell death without lysis. According to some embodiments, the CPE may be manifested as morphological changes in the host cell. Common examples of CPE include rounding of the infected cell, fusion with adjacent cells to form syncytia, and/or appearance of nuclear or cytoplasmic inclusion bodies.

As used herein, the term “transcript” refers to a RNA sequence. A transcript may be used as a template for protein synthesis.

As used herein, the terms “decoy transcript” and “parasitic pseudo-viral transcript” refers to a RNA transcript derived from the virus, the protection against which is sought, which transcript is capable of being replicated and packaged by the WV replication and packaging machinery.

The term “construct”, as used herein refers to an artificially assembled or isolated nucleic acid molecule which may be comprised of one or more nucleic acid sequences, wherein the nucleic acid sequences may be coding sequences (that is, sequence which encodes an end product, i.e. a protein), regulatory sequences, non-coding sequences, or any combination thereof. The term construct includes, for example, vectors, plasmids but should not be seen as being limited thereto.

As used herein, the term “decoy virion” refers to a viral particle which includes in its core the decoy transcript. According to some embodiments, the decoy virion may be assembled within the host cell. According to some embodiments, the decoy virion may be artificially assembled in which case it is referred to as a “virion transport vehicle”.

As used herein, the term “transport vehicle” refers to an agent suitable for efficient delivery of the hereindisclosed decoy transcript.

According to some embodiments, the transport vehicle may be a vector. As used herein the term “vector” refers to constructs engineered to encode or express polynucleotides in target cells, such as DNA, RNA, miRNA, shRNA, siRNA and antisense oligonucleotides. Vectors may include such vectors as, but not limited to, viral and non-viral vectors. The term “expression vector” refers to vectors that have the ability to incorporate and express heterologous nucleic acid fragments (such as DNA) in a foreign cell. In other words, an expression vector comprises nucleic acid sequences/fragments (such as DNA, mRNA, tRNA, rRNA), capable of being transcribed or expressed in a target cell. Many viral, prokaryotic and eukaryotic expression vectors are known and/or commercially available. Selection of appropriate expression vectors is within the knowledge of those having skill in the art.

According to some embodiments, the transport vehicle may be a liposome. As used herein, the term “liposomes” refer to microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains. The decoy transcript may be completely or partially located in the interior space of the liposome, within the bilayer membrane of the liposome, or associated with the exterior surface of the liposome membrane. The liposome may facilitate or assist in the delivery of the decoy transcript into a target cell. The liposome may also protect the nucleic acid from an environment which may contain enzymes or chemicals that degrade nucleic acids and/or systems or receptors that cause the rapid excretion of the nucleic acids.

According to some embodiments, the transport vehicle may be a nanoparticle. As used herein, a “nanoparticle” refers to a colloidal particle for delivery of a molecule or agent that is microscopic in size of between or about between 1 and 1000 nanometers (nm), such as between 1 and 100 nm and behave as a whole unit in terms of transport and properties. Nanoparticles include those that are uniform in size. Nanoparticles include those that contain a targeting molecule attached to the outside.

According to some embodiments, the nanoparticle may be a lipid nanoparticle. As used herein, the term “lipid nanoparticle” refers to a transfer vehicle comprising one or more lipids (e.g., cationic lipids, non-cationic lipids, and PEG-modified lipids). Preferably, the lipid nanoparticles are formulated to deliver decoy transcript into target cells. Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine.

According to some embodiments, the transport vehicle may be a virion. As used herein, the term “virion” refers to a capsid encapsulating the decoy transcript and capable of infecting host cells. According to some embodiments, the capsid may be the WV capsid while the inner core contains the decoy transcript.

The nucleic acids of the present disclosure may comprise one or more regions or parts which act or function as an untranslated region. The 5′ UTR starts at the transcription start site and continues to the start codon but does not include the start codon; whereas, the 3′ UTR starts immediately following the stop codon and continues until the transcriptional termination signal. The regulatory features of a UTR can be incorporated into the polynucleotides of the present disclosure to, among other things, enhance the stability of the molecule.

In some embodiments of the disclosure, the 5′UTR is derived from the WV. Alternatively, the 5′ UTR may be heterologous UTR, e.g. a UTR of a different virus. In another embodiment, a 5′ UTR is a synthetic UTR, i.e., does not occur in nature. Synthetic UTRs include UTRs that have been mutated. A synthetic UTR may have improved properties, e.g., increased gene expression.

Similarly, introduction, removal or modification of 3′ UTR AU rich elements (AREs) can be used to modulate the stability of nucleic acids (e.g., RNA) of the disclosure. The 3′ UTRs may be heterologous or synthetic, as described with respect to the 5′ UTR.

Those of ordinary skill in the art will understand that 5′UTRs that are heterologous or synthetic may be used with any desired 3′ UTR sequence. For example, a heterologous 5′UTR may be used with a synthetic 3′UTR with a heterologous 3″ UTR.

The UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series.

The herein disclosed transcript may be an RNA produced using an in vitro transcription (IVT) system, as known in the art. An in vitro transcription system typically comprises a transcription buffer, nucleotide triphosphates (NTPs), an RNase inhibitor and a polymerase. In some embodiments, the RNA transcript is generated using a non-amplified, linearized DNA template in an in vitro transcription reaction to generate the RNA transcript. Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase. In some embodiments, the RNA transcript is capped via enzymatic capping. In some embodiments, the RNA comprises 5′ terminal cap, for example, 7mG(5′)ppp(5′)NlmpNp.

In an embodiment the RNA transcript of the PSCT may be synthesized using a RdRp, in vivo or in vitro.

The transcript of the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

The transcript of the present disclosure may be synthesized by the addition of monomer building blocks and may be carried out in a liquid phase.

The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, Mass.), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

According to some embodiments, the herein disclosed decoy transcript may be administered in a composition (e.g., pharmaceutical compositions).

In some embodiments, the composition is in a form suitable for inhalation. In some embodiments, the form of the composition is selected from the group consisting of nose drops, nasal sprays, sprayable liquid composition, inhalants and throat sprays. In some embodiments, the composition is provided in pressurized aerosol dosage form.

In some embodiments, there is provided a device comprising a pressurized aerosol dosage form of any one of the compositions disclosed herein, and configured to produce aerosol from the pressurized aerosol dosage form. In some embodiments, the device is selected from the group consisting of nebulizer, MESH ultrasonic nebulizer, inhaler and atomizer. In some embodiments, said device is a hand-held device. Each possibility is a separate embodiment.

In some embodiments, said administering is via inhalation, e.g. utilizing apparatus configured to produce vapor and/or aerosol.

Inhalation administration can include an intranasal spray. Various forms suitable for administration by inhalation include aerosols, mists or powders. Each possibility is a separate embodiment.

Pharmaceutical compositions comprising the composition disclosed herein can be delivered in the form of an aerosol spray presentation from pressurized packaging or a nebulizer, e.g. with the use of a propellant (e.g., dichlorodifluoromethane, trichlorofluoromethane, dichlorotetrafluoroethane, carbon dioxide, or the like).

In some embodiments, the aerosol dosage form is encapsulated in a cartridge or a capsule.

In some embodiments, the composition disclosed herein is in a form of capsules or cartridges for use within a pressurized delivery system.

In some embodiments, the composition disclosed herein can be formulated into liquid compositions sprayable from non-aerosol packaging.

The term “aerosol” as used herein refers to a suspension of liquid or solid particles in a gas. Typically, the diameter of the droplets (also referred to as particle) is within the range of 10⁻⁹ to 10⁻⁴ m.

In some embodiment, the composition is suitable for administration via an oxygen machine, oxygen inhaler, oxygen bag or oxygen cylinder. In some embodiment, inhalation is performed via a face mask connected to the apparatus. In some embodiment, the composition is provided in parallel to oxygen.

In some embodiments, the antiviral composition is for systemic use such as for intravenous, intramuscular, or intraperitoneal injection. Each possibility is a separate embodiment.

According to some embodiments, the hereindisclosed decoy transcript, may be used to treat a viral infection. According to some embodiments, the hereindisclosed decoy transcript, may be used to prevent spread of a viral infection. According to some embodiments, the hereindisclosed decoy transcript, may be used to attenuate a viral infection.

According to some embodiments, the hereindisclosed decoy transcript, may be administered to a subject (e.g., a mammalian subject, such as a human subject) in an effective amount.

The present disclosure provides methods comprising administering the decoy transcript to a subject in need thereof. The exact amount required may vary from subject to subject, depending on the species, age, and general condition of the subject, the severity of the disease, the particular composition, its mode of administration, its mode of activity, and the like. The specific therapeutically effective, prophylactically effective, or appropriate dose level enabling population wide effect may depend upon a variety of factors including the disorder being treated and the severity of the disorder; the specific composition employed; the age, body weight, general health, sex and diet of the subject; the time of administration, route of administration, drugs used in combination or coincidental with the specific compound employed; and like factors well known in the medical arts.

As used herein, the singular forms “a,” “an” and “the” include plural referents unless the context clearly dictates otherwise.

As used herein, ranges and amounts can be expressed as “about” or “approximately” a particular value or range. “About” or “approximately” also includes the exact amount. Generally, “about” includes an amount that would be expected to be within experimental error.

As used herein, “about the same” means within an amount that one of skill in the art would consider to be the same or to be within an acceptable range of error. For example, typically, for pharmaceutical compositions, within at least 1%, 2%, 3%, 4%, 5% or 10% is considered about the same. Such amounts can vary depending upon the tolerance for variation in the particular composition by subjects.

As used herein, “optional” or “optionally” means that the subsequently described event or circumstance does or does not occur, and that the description includes instances where said event or circumstance occurs and instances where it does not.

### Embodiments of the Invention

According to some embodiments, there is provided a parasitic pseudo-viral transcript (PSCT), which when administered to a subject is capable of treating, attenuating and/or preventing spread of a virus-induced infection and/or disease. The decoy transcript is derived from a ssRNA virus (WV), and includes the 5′UTR (or a part thereof, a homolog thereof and/or a modified version thereof) of the of the WV, the genomic packaging signal (GPS) of the WV (or a part thereof, a homolog thereof and/or a modified version thereof), the 3′UTR of the WV, at least one extrinsic, non-naturally occurring and/or artificially introduced stop codon, and the poly-A tail (or a part thereof, a homolog thereof and/or a modified version thereof) and. It is understood that the stop codon used may be any of the UAG, UAA, UGA stop codons. As such when one or more specific stop codons are disclosed as part of the sequences set forth herein, such stop codons may be substituted it may be substituted with any other stop codon of the genetic code.

In an embodiment, the at least one exogenous stop codon is positioned at any position throughout the sequence of the decoy transcript. In an embodiment, the decoy transcript does not produce any WV proteins. In an embodiment, the decoy transcript produces a short peptide. In an embodiment, the short peptide resulting from the decoy transcript is a nonfunctional peptide.

According to some embodiments, decoy transcript may be stabilized. According to some embodiments, the 5′ end of the decoy transcript may be capped.

According to some embodiments, the herein disclosed transcript may be stabilized. As used herein a “stabilized RNA” molecule may refer to RNA molecules that can contain stabilizing elements, including, but not limited to a 5′-cap structure or a 3′-poly(A) tail.

The 5′-capping of polynucleotides may be completed concomitantly during the in vitro-transcription reaction e.g. using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-0 methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-0 methyl-transferase. Cap 3 structure may be generated from the Cap 2 structure followed by the 2′-O-methylation of the 5′-preantepenultimate nucleotide using a 2′-0 methyl-transferase. Each possibility is a separate embodiment. According to some embodiments, the capping comprises a 7-methylguanosine cap (m7G) or a m7G-analog.

According to some embodiments, the 5′UTR may be capped using CleanCap® TriLink co-transcriptional capping method. This may result in the naturally occurring Cap 1 structure.

According to some embodiments, the transcript may also be modified with 5-methoxyuridine. Without being bound by any theory this may provide optimized stability of the transcripts for mammalian systems, while mimicking WV mRNA.

According to some embodiments, the 3′ UTR includes a 3′-poly(A) tail. The 3′-poly(A) tail is typically a stretch of adenosine nucleotides added to the 3′-end of the transcribed RNA. It can, in some instances, comprise up to about 400 adenosine nucleotides. In some embodiments, the length of the 3′-poly(A) tail may be an essential element with respect to the stability of the individual RNA.

According to some embodiments, the decoy transcript includes a nucleotide sequence being identical to or having at least 80%, at least 90% at least 95% or at least 99% sequence similarity to the nucleotide sequence set forth in SEQ ID NO: 2 or SEQ ID NO: 6 (decoy transcripts). See table 1 below (start and stop codons are highlighted in bold). It is understood that the stop codon may be modified according to the genetic code, as further elaborated herein).

According to some embodiments, the decoy transcript includes a nucleotide sequence being identical to or having at least 80%, at least 90% at least 95% or at least 99% sequence similarity to the nucleotide sequence set forth in SEQ ID NO: 3 or SEQ ID NO: 7 (5′UTR of decoy transcripts). See table 1 below.

According to some embodiments, the decoy transcript includes a nucleotide sequence being identical to or having at least 80%, at least 90% at least 95% or at least 99% sequence similarity to the nucleotide sequence set forth in SEQ ID NO: 4 (GPS of decoy transcript). See table 1 below.

According to some embodiments, the decoy transcript includes a nucleotide sequence being identical to or having at least 80%, at least 90% at least 95% or at least 99% sequence similarity to the nucleotide sequence set forth in SEQ ID NO: 5 (3′UTR of decoy transcript).

TABLE 1
decoy transcript related sequences

| SEQ ID NO: | Description | Sequence |
|---|---|---|
| 2 | Full length decoy | AUUAAAGGUUUAUACCUUCCCAGGUAACAAACC |
| | transcript | AACCAACUUUCGAUCUCUUGUAGAUCUGUUCUC |
| | | UAAACGAACUUUAAAAUCUGUGUGGCUGUCAC |
| | | UCGGCUGCAUGCUUAGUGCACUCACGCAGUAUA |
| | | AUUAAUAACUAAUUACUGUCGUUGACAGGACA |
| | | CGAGUAACUCGUCUAUCUUCUGCAGGCUGCUUA |
| | | CGGUUUCGUCCGUGUUGCAGCCGAUCAUCAGCA |
| | | CAUCUAGGUUUCGUCCGGGUGUGACCGAAAGGU |
| | | AAGAUGGAGAGCCUUGUCCCUGGUUUCAACGA |
| | | GAAAACACACGUCCAACUCAGUUUGCCUGUUUU |
| | | ACAGGUUCGCGACGUGCUCGUACGUGGCUUUGG |
| | | AGACUCCGUGGAGGAGGUCUUAUCAGAGGCACG |
| | | UCAACAUCUUAAAGAUGGCACUUGUGGCUUAG |
| | | UAGAAGUUGAAAAAGGCGUUUUGCCUCAACUU |
| | | GAACAGCCCUAUGUGUUCUAGAUAGAUAGGAC |
| | | ACUUUGAUGGACAACAGGGUGAAGUACCAGUU |
| | | UCUAUCAUUAAUAACACUGUUUACACAAAAGU |
| | | UGAUGGUGUUGAUGUAGAAUUGUUUGAAAAUA |
| | | AAACAACAUUACCUGUUAAUGUAGCAUUUGAG |
| | | CUUUGGGCUAAGCGCAACAUUAAACCAGUACCA |
| | | GAGGUGAAAAUACUCAAUAAUUUGGGUGUGGA |
| | | CAUUGCUGCUAAUACUGUGAUCUGGGACUACAA |
| | | AAGAGAUGCUCCAGCACAUAUAUCUACUAUUGG |
| | | UGUUUGUUCUAUGACUGACAUAGCCAAGAAACC |
| | | AACUGAAACGAUUUGUGCACCACUCACUGUCUU |
| | | UUUUGAUGGUAGAGUUGAUGGUCAAGUAGACU |
| | | UAUUUAGAAAUGCCCGUAAUGGUGUUCUUAUU |
| | | ACAGAAGGUAGUGUUAAAGGUUUACAACCAUC |
| | | UGUAGGUCCCAAACAAGCUAGUCUUAAUGGAG |
| | | UCACAUUAAUUGGAGAAGCCGUAAAAACACAG |
| | | UUCAAUUAUUAUAAGAAAGUUGAUGGUGUUGU |
| | | CCAACAAUUACCUGAAACUUACUUUACUCAGAG |
| | | UAGAAAUUUACAAGAAUUUAAACCCAGGAGUC |
| | | AAAUGGAAAUUGAUUUCUUAGAAUUAGCUAUG |
| | | GAUGAAUUCAUUGAACGGUAUAAAUUAGAAGG |
| | | CUAUGCCUUCGAACAUAUCGUUUAUGGAGAUU |
| | | UUAGUCAUAGUCAGUUAGGUGGUUUACAUCUA |
| | | CUGAUUGGACUAGCAAUCUCACAUAGCAAUCUU |
| | | UAAUCAGUGUGUAACAUUAGGGAGGACUUGAA |
| | | AGAGCCACCACAUUUUCACCGAGGCCACGCGGA |
| | | GUACGAUCGAGUGUACAGUGAACAAUGCUAGG |
| | | GAGAGCUGCCUAUAUGGAAGAGCCCUAAUGUG |
| | | UAAAAUUAAUUUUAGUAGUGCUAUCCCCAUGU |
| | | GAUUUUAAUAGCUUCUUAGGAGAAUGACAAAA |
| | | AAAAAAAAAAAAAAAA |
| 3 | 5’UTR of decoy | AUUAAAGGUUUAUACCUUCCCAGGUAACAAACC |
| | transcript | AACCAACUUUCGAUCUCUUGUAGAUCUGUUCUC |
| | | UAAACGAACUUUAAAAUCUGUGUGGCUGUCAC |
| | | UCGGCUGCAUGCUUAGUGCACUCACGCAGUAUA |
| | | AUUAAUAACUAAUUACUGUCGUUGACAGGACA |
| | | CGAGUAACUCGUCUAUCUUCUGCAGGCUGCUUA |
| | | CGGUUUCGUCCGUGUUGCAGCCGAUCAUCAGCA |
| | | CAUCUAGGUUUCGUCCGGGUGUGACCGAAAGGU |
| | | AAGAUGGAGAGCCUUGUCCCUGGUUUCAACGAG |
| | | AAAACACACGUCCAACUCAGUUUGCCUGUUUUA |
| | | CAGGUUCGCGACGUGCUCGUACGUGGCUUUGGA |
| | | GACUCCGUGGAGGAGGUCUUAUCAGAGGCACGU |
| | | CAACAUCUUAAAGAUGGCACUUGUGGCUUAGU |
| | | AGAAGUUGAAAAAGGCGUUUUGCCUCAACUUG |
| | | AACAGCCCUAUGUGUUC |
| 4 | GPS of decoy | GACACUUUGAUGGACAACAGGGUGAAGUACCA |
| | transcript | GUUUCUAUCAUUAAUAACACUGUUUACACAAA |
| | | AGUUGAUGGUGUUGAUGUAGAAUUGUUUGAAA |
| | | AUAAAACAACAUUACCUGUUAAUGUAGCAUUU |
| | | GAGCUUUGGGCUAAGCGCAACAUUAAACCAGUA |
| | | CCAGAGGUGAAAAUACUCAAUAAUUUGGGUGU |
| | | GGACAUUGCUGCUAAUACUGUGAUCUGGGACU |
| | | ACAAAAGAGAUGCUCCAGCACAUAUAUCUACUA |
| | | UUGGUGUUUGUUCUAUGACUGACAUAGCCAAG |
| | | AAACCAACUGAAACGAUUUGUGCACCACUCACU |
| | | GUCUUUUUUGAUGGUAGAGUUGAUGGUCAAGU |
| | | AGACUUAUUUAGAAAUGCCCGUAAUGGUGUUC |
| | | UUAUUACAGAAGGUAGUGUUAAAGGUUUACAA |
| | | CCAUCUGUAGGUCCCAAACAAGCUAGUCUUAAU |
| | | GGAGUCACAUUAAUUGGAGAAGCCGUAAAAAC |
| | | ACAGUUCAAUUAUUAUAAGAAAGUUGAUGGUG |
| | | UUGUCCAACAAUUACCUGAAACUUACUUUACUC |
| | | AGAGUAGAAAUUUACAAGAAUUUAAACCCAGG |
| | | AGUCAAAUGGAAAUUGAUUUCUUAGAAUUAGC |
| | | UAUGGAUGAAUUCAUUGAACGGUAUAAAUUAG |
| | | AAGGCUAUGCCUUCGAACAUAUCGUUUAUGGA |
| | | GAUUUUAGUCAUAGUCAGUUAGGUGGUUUACA |
| | | UCUACUGAUUGGACUAGC |
| 5 | 3’UTR of decoy | AAUCUCACAUAGCAAUCUUUAAUCAGUGUGUA |
| | transcript | ACAUUAGGGAGGACUUGAAAGAGCCACCACAUU |
| | | UUCACCGAGGCCACGCGGAGUACGAUCGAGUGU |
| | | ACAGUGAACAAUGCUAGGGAGAGCUGCCUAUA |
| | | UGGAAGAGCCCUAAUGUGUAAAAUUAAUUUUA |
| | | GUAGUGCUAUCCCCAUGUGAUUUUAAUAGCUUC |
| | | UUAGGAGAAUGACAAAAAAAAAAAAAAAAAAA |
| | | A |
| 6 | Short full length | AUUAAAGGUUUAUACCUUCCCAGGUAACAAACC |
| | decoy transcript | AACCAACUUUCGAUCUCUUGUAGAUCUGUUCUC |
| | | UAAACGAACUUUAAAAUCUGUGUGGCUGUCAC |
| | | UCGGCUGCAUGCUUAGUGCACUCACGCAGUAUA |
| | | AUUAAUAACUAAUUACUGUCGUUGACAGGACA |
| | | CGAGUAACUCGUCUAUCUUCUGCAGGCUGCUUA |
| | | CGGUUUCGUCCGUGUUGCAGCCGAUCAUCAGCA |
| | | CAUCUAGGUUUCGUCCGGGUGUGACCGAAAGGU |
| | | AAGAUGGAGAGCCUUGUCCCUGGUUUCAACGA |
| | | GAAAACACACGUCCAACUCAGUUUGCCUGUUUU |
| | | ACAGGUUCGCGACGUGCUCGUACGUGGCUUUGG |
| | | AGACUCCGUGGAGGAGGUCUUAUCAGAGGCACG |
| | | UCAACAUCUUAAAGAUGGCACUUGUGGCUUAG |
| | | UAGAAGUUGAAAAAGGCGUUUUGCCUCAACUU |
| | | GAACAGCCCUAUGUGUUCUAGAUAGAUAGGAC |
| | | ACUUUGAUGGACAACAGGGUGAAGUACCAGUU |
| | | UCUAUCAUUAAUAACACUGUUUACACAAAAGU |
| | | UGAUGGUGUUGAUGUAGAAUUGUUUGAAAAUA |
| | | AAACAACAUUACCUGUUAAUGUAGCAUUUGAG |
| | | CUUUGGGCUAAGCGCAACAUUAAACCAGUACCA |
| | | GAGGUGAAAAUACUCAAUAAUUUGGGUGUGGA |
| | | CAUUGCUGCUAAUACUGUGAUCUGGGACUACAA |
| | | AAGAGAUGCUCCAGCACAUAUAUCUACUAUUGG |
| | | UGUUUGUUCUAUGACUGACAUAGCCAAGAAACC |
| | | AACUGAAACGAUUUGUGCACCACUCACUGUCUU |
| | | UUUUGAUGGUAGAGUUGAUGGUCAAGUAGACU |
| | | UAUUUAGAAAUGCCCGUAAUGGUGUUCUUAUU |
| | | ACAGAAGGUAGUGUUAAAGGUUUACAACCAUC |
| | | UGUAGGUCCCAAACAAGCUAGUCUUAAUGGAG |
| | | UCACAUUAAUUGGAGAAGCCGUAAAAACACAG |
| | | UUCAAUUAUUAUAAGAAAGUUGAUGGUGUUGU |
| | | CCAACAAUUACCUGAAACUUACUUUACUCAGAG |
| | | UAGAAAUUUACAAGAAUUUAAACCCAGGAGUC |
| | | AAAUGGAAAUUGAUUUCUUAGAAUUAGCUAUG |
| | | GAUGAAUUCAUUGAACGGUAUAAAUUAGAAGG |
| | | CUAUGCCUUCGAACAUAUCGUUUAUGGAGAUU |
| | | UUAGUCAUAGUCAGUUAGGUGGUUUACAUCUA |
| | | CUGAUUGGACUAGCAAUCUCACAUAGCAAUCUU |
| | | UAAUCAGUGUGUAACAUUAGGGAGGACUUGAA |
| | | AGAGCCACCACAUUUUCACCGAGGCCACGCGGA |
| | | GUACGAUCGAGUGUACAGUGAACAAUGCUAGG |
| | | GAGAGCUGCCUAUAUGGAAGAGCCCUAAUGUG |
| | | UAAAAUUAAUUUUAGUAGUGCUAUCCCCAUGU |
| | | GAUUUUAAUAGCUUCUUAGGAGAAUGACAAAA |
| | | AAAAAAAAAAAAAAAA |
| 7 | Short 5’UTR of | AUUAAAGGUUUAUACCUUCCCAGGUAACAAACC |
| | decoy transcript | AACCAACUUUCGAUCUCUUGUAGAUCUGUUCUC |
| | | UAAACGAACUUUAAAAUCUGUGUGGCUGUCAC |
| | | UCGGCUGCAUGCUUAGUGCACUCACGCAGUAUA |
| | | AUUAAUAACUAAUUACUGUCGUUGACAGGACA |
| | | CGAGUAACUCGUCUAUCUUCUGCAGGCUGCUUA |
| | | CGGUUUCGUCCGUGUUGCAGCCGAUCAUCAGCA |
| | | CAUCUAGGUUUCGUCCGGGUGUGACCGAAAGGU |
| | | AAGAUGGAGAGCCUUGUCCCUGGUUUCAACGA |
| | | GAAAACACACGUCCAACUCAGUUUGCCUGUUUU |
| | | ACAGGUUCGCGACGUGCUCGUACGUGGCUUUGG |
| | | AGACUCCGUGGAGGAGGUCUUAUCAGAGGCACG |
| | | UCAACAUCUUAAAGAUGGCACUUGUGGCUUAG |
| | | UAGAAGUUGAAAAAGGCGUUUUGCCUCAACUU |
| | | GAACAGCCCUAUGUGUUC |
| 8 | Stop codon repeat | UAGAUAGAUAG |

According to some embodiments, the decoy transcript may be derived from the SARS-CoV having the nucleotide sequence set forth in SEQ ID NO: 9 (NC_004718.3) or any variant or mutant strain thereof.

According to some embodiments, the decoy transcript may be derived from the MERS-CoV virus having the nucleotide sequence set forth in SEQ ID NO: 10. (NC_019843.3) or any variant or mutant strain thereof.

According to some embodiments, the decoy transcript may be derived from ebola virus having the nucleotide sequence set forth in SEQ ID NO: 11 (NC_002549.1) or any variant or mutant strain thereof.

According to some embodiments, the decoy transcript may be derived from the flaviviruses such as dengue virus having the nucleotide sequence set forth in SEQ ID NO: 12 (NC_001477.1) or West Nile fever virus having the nucleotide sequence set forth in SEQ ID NO: 13 (Accession: M12294.2 GI: 11497619).

According to some embodiments, the stop codon is positioned between the 5′UTR and the GPS. According to some embodiments, the stop codon is positioned within the 5′UTR. According to some embodiments, the stop codon is positioned within the GPS. According to some embodiments, the stop codon is positioned between the GPS and the 3′UTR. According to some embodiments, the stop codon is positioned a few nucleotides (e.g. 2-5 nucleotides) from a start codon of the WV, so as to ensure rapid disassembly of ribosomes from the decoy transcript.

According to some embodiments, the decoy transcript further includes one or more additional stop codons, in frame shift to the first stop codon. According to some embodiments, the decoy transcript further includes one or more additional GPS sequences (or parts thereof) for enhancing N-protein binding.

According to some embodiments, the decoy transcript further includes one or more sequences complementary to sequences in the WV genome, preferably before the 3′UTR of the transcript. The complementary sequences (also referred to herein as “antisense sequences”) may bind the WV and thereby interfere with WV replication. The sequences are designed such that they will interfere with the replication of the viral genome while having minimal impact on the replication of the decoy transcript. According to some embodiments, the antisense sequence pairs with sequences of the WV gRNA at genomic regions that do not encode for vital proteins so as not to interfere with the production of proteins required for the assembly of decoy virions. According to some embodiments, the antisense sequences are each flanked by a leader and a transcription regulatory sequence (TRS) of the virus, so as to enhance transcription thereof.

According to some embodiments, the WV from which the decoy transcript is derived has a viral genome of between 20-40 kilobases. This may advantageously ensure that the replication of the decoy transcript is significantly faster than that of the WV, thereby providing a competitive advantage.

According to some embodiments, the ratio in nucleotide length of the decoy transcript vis-à-vis the WV is at least 1:5 (i.e. the WV being at least 5 times longer than the decoy transcript. According to some embodiments, the ratio in nucleotide length of the decoy transcript vis-à-vis the WV is at least 1:10 (i.e. the WV being at least 10 times longer than the decoy transcript. According to some embodiments, the ratio in nucleotide length of the decoy transcript vis-à-vis the WV is at least 1:15 (i.e. the WV being at least 15 times longer than the decoy transcript. According to some embodiments, the ratio in nucleotide length of the decoy transcript vis-à-vis the WV is at least 1:20 (i.e. the WV being at least 20 times longer than the decoy transcript.

According to some embodiments, the decoy transcripts is about 500-2500 or about 1000-2000 nucleotides (nt) long. According to some embodiments, the decoy transcripts is about 1500 nt long.

According to some embodiments, the WV from which the decoy transcript is derived is a zoonotic virus. According to some embodiments, the WV from which the decoy transcript is derived is a positive-strand ssRNA virus.

According to some embodiments, the WV from which the decoy transcript is derived is a corona virus. According to some embodiments, the corona virus is a Severe acute respiratory syndrome coronavirus (SARS), Severe acute respiratory syndrome coronavirus 2 (SARS-CoV-2 virus) or a Middle East respiratory syndrome-related coronavirus (MERS). Each possibility is a separate embodiment. It is understood that other viruses including variants of the above-mentioned viruses and/or presently unknown viruses may likewise be targeted by decoy transcript, as essentially described herein.

According to some embodiments, the WV maybe any one of the following pathogenic positive-sense ssRNA viruses: Coronaviridae family, hepatitis c virus, West Nile virus, dengue virus, and the common cold rhinoviruses.

According to some embodiments, the WV maybe any one of the following pathogenic negative-strand ssRNA viruses: RSV, parainfluenza viruses, influenza viruses, Ebola virus, Marburg virus and the like.

According to some embodiments, the decoy transcript may be “custom-made” according to the virus the targeting of which is desired. According to some embodiments, the decoy transcript may be constructed based on the genomic sequence of the virus the targeting of which is desired.

According to some embodiments, the decoy transcript is a purified RNA molecule. As such the decoy transcript may be suitable for direct delivery e.g. by aerosolizing the transcript diluted in water or other suitable carrier.

According to some embodiments, the decoy transcript may be incorporated into a transcription vector, such as but not limited to an adeno-viral vector or a lentiviral vector.

According to some embodiments, the vector includes a promoter transcriptionally associated with the decoy transcript. According to some embodiments, the promoter may be a constitutive promoter. According to some embodiments, the promoter may be an inducible promoter. According to some embodiments, the promoter may be a tissue specific promoter. According to some embodiments, the tissue specific promoter may be a lung tissue specific promoter.

According to some embodiments, the decoy transcript may be encapsulated in a viral infectious particle (also referred to herein as a decoy virion transport vehicle).

According to some embodiments, the decoy transcript may be encapsulated, attached to or otherwise associated with a liposome or a lipid nanoparticle. According to some embodiments, the encapsulated decoy transcript may be suitable for administration by injection, e.g. intravenous injection.

According to some embodiments, the decoy transcript may be formulated for oral and/or nasal administration. According to some embodiments, the decoy transcript may be formulated for administration via inhalation. In some embodiments, the form of the antiviral composition is selected from the group consisting of nose drops, nasal sprays, sprayable liquid composition, inhalants and throat sprays.

According to some embodiments, the decoy transcript may be formulated for administration via injection or other delivery method known in the art.

According to some embodiments, there is provided a method for treating, attenuating severity of and/or inhibiting spread of a ssRNA virus in an individual subject or a subject population. According to some embodiments, the method includes administering the decoy transcript disclosed herein to a subject. This may provide relief to the treated subject (treatment or amelioration of symptoms), which is referred to herein as “direct treatment”. In addition, the treatment may also indirectly treat individuals surrounding the subject (family, co-workers etc.) which is referred to herein as “indirect treatment” through transmission to surrounding individuals with decoy virions produced in PSCT-treated subjects. According to some embodiments, the decoy transcript is only effective in subjects infected with the WV and will be destroyed in cells which are not affected. Accordingly, the decoy transcript is suitable for direct or indirect treatment of subjects infected with the WV from which the decoy transcript is derived. The decoy transcript is therefore particularly effective for treating large population of infected individuals, such as during pandemics. It is further understood that the spread of the WV may be attenuated due to the fact that the WV load of the treated subjects (whether directly or indirectly) is substantially reduced.

The following examples are included to demonstrate examples of certain preferred embodiments of the invention. It should be appreciated by those of skill in the art that the techniques disclosed in the examples that follow represent approaches the inventors have found function well in the practice of the invention, and thus can be considered to constitute examples of preferred modes for its practice. However, those of skill in the art should, in light of the present disclosure, appreciate that many changes can be made in the specific embodiments that are disclosed and still obtain a like or similar result without departing from the spirit and scope of the invention.

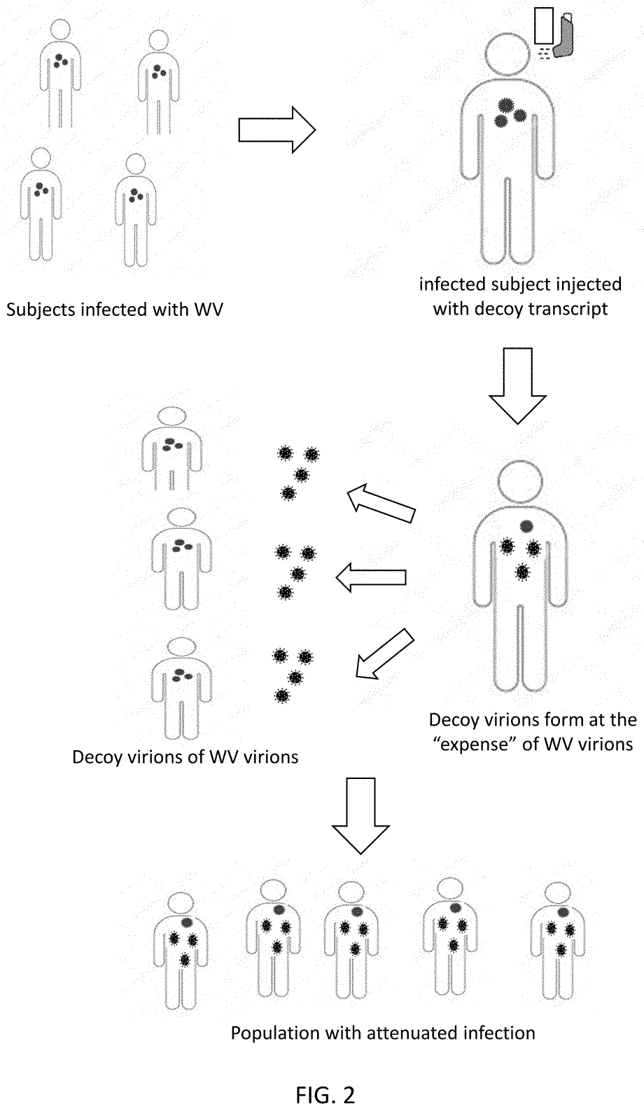

Reference is now made to FIG. 2, which illustratively depicts the steps of direct and indirect decoy transcript treatment.

Initially viral infection in a population is identified. Once identified, one or more individuals in the population may be administered the decoy transcript. The administration form may be by inhalation, nebulization, oral administration (e.g. as a liquid), injection etc. The subject administered with the decoy transcript may be random (e.g. the first one to enter a clinic). Alternatively, the subject may be chosen based on risk factors, viral load, age, activity (e.g. how likely he/she is to encounter additional infected subjects) and the like.

Once an infected subject is administered with the decoy transcript, the RdRp of the WV will recognize the decoy transcript as a WV transcript and initiates its replication. Optionally, in case the decoy transcripts include antisense sequences, these will be transcribed and serve as inhibitors of WV gRNA replication.

The replicated decoy transcripts will be recognized by the WV packaging machinery resulting in the production of decoy virions. Due to the rapid replication of the decoy transcript, the decoy transcript limits the amount of RdRp-, N- and M-proteins available to the WV transcript and thereby slows WV replication and assembly. This results in many more decoy virions being formed as opposed to WV virions and an attenuation (and optionally even elimination) of the WV infection in the treated subject.

Once the treated subject encounters other infected subjects, the decoy virions will be co-transmitted with the WV to other infected individuals, thereby reducing the overall WV-load in the population.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### EXAMPLES

### Example 1— Manufacturing of PSCT Constructs

The PSCT is manufactured commercially by TriLink BioTechnologies, San Diego, Calif. First, a refined proprietary method of solid-phase chemical synthesis of oligonucleotide DNA synthesis will be employed to obtain the core sequence of the PSCT. Next, the linearized DNA sequence is used as a template to produce capped RNAs via T7 RNA polymerase synthesis. The in vitro RNA transcription manufactures a capped and polyadenylated messenger RNAs (mRNAs), with co-transcriptional capping achieved by the proprietary CleanCap® technology. Additionally, the poly-A-tail is added using poly-A polymerase.

### Example 2—Virus Handling and Titration

Virus titer in the frozen culture supernatant was determined by using a standard plaque assay. Briefly, 100 μL of virus in 10-fold serial dilution was added, in duplicates, to a monolayer of Vero E6 cells in a 24-well plate. After 1 h of incubation at 37° C. the viral inoculum was aspirated, and 1 mL of carboxymethylcellulose (CMC) overlay with DMEM medium (or 199), supplemented with 5% FCS, was added to each well. After 2-4 days of culture, the plaques were visualized and counted by standard crystal violet staining, and the virus titer in plaque-forming units per mL (PFU/mL) was calculated.

### Example 3—Preparation of mRNA-Lipid Complexes and Transfection Procedure

mRNA-lipid complexes are prepared using Lipofectamine™ MessengerMAX transfection reagent (Thermo Fisher Scientific Inc.), as recommended by the manufacturer as a starting point for optimization. Next mRNA-lipid complexes are added to Vero E6 cells plated in a 96-well plate; all in accordance with manufacturer recommendations.

### Example 4—Plaque Reduction Assay

Trypsinized Vero E6 cells are resuspended in growth medium and plated at 20,000 cells per well in 96-well plates, one hour later the cultures are preincubated with synthetic SARS2 decoy mRNA-lipid complexes previously prepared (0.3 μL of lipofectamine MessengerMAX™ reagent premixed with 100 ng of relevant mRNA per well). EGFP mRNA-lipid complex is used as a positive control to monitor transfection efficacy (all mRNAs from TriLink BioTechnologies). Cultures treated with transfection reagent alone (vehicle) serve as negative control for the plaque reduction assay.

The inhibitory effects of the SARS2 decoy mRNA and controls will be tested in triplicate wells in 96-well plates. Following 6-12 hours of incubation, the medium is aspirated, and 100 μL of virus will be added to each well at a titer of 100 PFU/well. After 1 h incubation, the virus inoculum is aspirated, and a CMC-overlay containing standard maintenance medium will be added. After 3 days in culture, the plates are fixed and stained with crystal violet and the number of plaques is counted visually. The % inhibition of plaques in each well is determined as previously described.

### Example 5—Mortality in IBCoV and PSCT Infected Chickens

A PSCT for poultry infectious bronchitis CoV (IBCoV) is synthesized.

Twenty chickens are infected with D3 CoV.

PSCT is administered to ten chickens, before and/or after manifestation of disease symptoms.

Ten chickens are either left untreated or treated with a control transcript incapable of being packaged into viral particles.

Chicken mortality induced by the viral infection is compared between the two groups.

### Example 6—Mortality in MHV Infected Mice

A PSCT for murine CoV (MHV) is synthesized.

Twenty mice are infected with MHV.

PSCT is administered to ten mice, before and/or after manifestation of disease symptoms.

Ten mice are either left untreated or treated with a control transcript incapable of being packaged into viral particles.

Mice mortality induced by the viral infection is compared between the two groups.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1.-45. (canceled)

46. A viral decoy transcript derived from a ssRNA virus (WV), the transcript comprising a 5′ end comprising a 5′UTR of the WV, a genomic packaging signal (GPS) of the WV, a 3′UTR of the WV, an extrinsic stop codon, and a poly-A tail.

47. The viral decoy transcript of claim 46, wherein the decoy transcript does not encode a WV RdRp and/or WV N-protein.

48. The viral decoy transcript of claim 46, wherein the decoy transcript does not encode any WV protein.

49. The decoy transcript of claim 46, comprising a nucleotide sequence having at least 80% sequence similarity to the nucleotide sequence set forth in SEQ ID NO: 2 or 6.

50. The decoy transcript of claim 46, comprising a nucleotide sequence having at least 80% sequence similarity to any one or more of the nucleotide sequences set forth in SEQ ID NO: 3-5 and 7.

51. The decoy transcript of claim 46, being derived from any one of the SEQ ID NO: 1 and 9-13.

52. The decoy transcript of claim 46, further comprising one or more additional stop codons, in frame shift to the first stop codon and one or more additional GPS sequences.

53. The decoy transcript of claim 46, further comprising one or more short WV specific antisense sequence, wherein the antisense sequence is flanked by at least one of a leader and a transcription regulatory sequence (TRS).

54. The decoy transcript of claim 46, wherein the genome of the WV from which the decoy transcript is derived comprises at least 20,000 nucleotides.

55. The decoy transcript of claim 46, wherein a ratio in the length of the decoy transcript relative to the WV is at least 1:15.

56. The decoy transcript of claim 46, wherein the WV from which the decoy transcript is derived is a zoonotic virus.

57. The decoy transcript of claim 46, wherein the WV from which the decoy transcript has a viral genome of 20-40 kilobases.

58. The decoy transcript of any one of claim 46, wherein the WV from which the decoy transcript is derived is a positive-sense single stranded RNA virus.

59. The decoy transcript of claim 46, wherein the decoy transcript is an isolated RNA molecule.

60. A composition comprising the decoy transcript of claim 46, and a suitable transport vehicle and/or carrier.

61. The composition of claim 60, wherein the carrier is water.

62. The composition of claim 60, wherein the transport vehicle is an in-vitro generated virion a liposome or a lipid nanoparticle.

63. The composition of claim 60, formulated for oral and/or nasal administration or for administration via inhalation.

64. A method for treating, attenuating and/or inhibiting spread of a ssRNA viral infection in a subject, the method comprising providing to the subject the composition of claim 60.

65. The method of claim 64, wherein the treated subject is a subject infected with the WV.
